SEQUENCE LISTING (203 sequences)

SEQ ID NO: 1 - protein, 120 aa, synthetic construct [sequence omitted]

SEQ ID NO: 2 - protein, 111 aa, synthetic construct [sequence omitted]

SEQ ID NO: 3 - protein, 113 aa, synthetic construct [sequence omitted]

SEQ ID NO: 4 - protein, 107 aa, synthetic construct [sequence omitted]

SEQ ID NO: 5 - protein, 118 aa, synthetic construct
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSS

SEQ ID NO: 6 - protein, 107 aa, synthetic construct
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIK

SEQ ID NO: 7 - protein, 115 aa, synthetic construct
QVQLVQSGAEVKKPGSSVKVSCKASGFTFTTYYISWVRQAPGQGLEYLGYINMGSGGTNYNEKFKGRVTITADKSTSTAYMELSSLRSEDTAVYYCAIIGYFDYWGQGTMVTVSS

SEQ ID NO: 8 - protein, 112 aa, synthetic construct
DVVMTQSPLSLPVTLGQPASISCRSSQSLLDSDGGTYLYWFQQRPGQSPRRLIYLVSTLGSGVPDRFSGSGSGTDFTLKISRVEAEDVGVYYCMQLTHWPYTFGQGTKLEIK

SEQ ID NO: 9 - protein, 125 aa, synthetic construct [sequence omitted]

SEQ ID NO: 10 - protein, 112 aa, synthetic construct [sequence omitted]

SEQ ID NO: 11 - protein, 116 aa, synthetic construct
EVQLVESGGGLVQPGGSLRLSCAASGFTFSSYMMSWVRQAPGKGLEWVATISGGGANTYYPDSVKGRFTISRDNAKNSLYLQMNSLRAEDTAVYYCARQLYYFDYWGQGTTVTVSS

SEQ ID NO: 12 - protein, 107 aa, synthetic construct
DIQMTQSPSSLSASVGDRVTITCLASQTIGTWLTWYQQKPGKAPKLLIYTATSLADGVPSRFSGSGSGTDFTLTISSLQPEDFATYYCQQVYSIPWTFGGGTKVEIK

SEQ ID NO: 13 - protein, 120 aa, synthetic construct
QVQLVQSGAEVKKPGSSVKVSCKASGGTFSSYAISWVRQAPGQGLEWMGLIIPMFDTAGYAQKFQGRVAITVDESTSTAYMELSSLRSEDTAVYYCARAEHSSTGTFDYWGQGTLVTVSS

SEQ ID NO: 14 - protein, 107 aa, synthetic construct
DIQMTQSPSSVSASVGDRVTITCRASQGISSWLAWYQQKPGKAPKLLISAASSLQSGVPSRFSGSGSGTDFTLTISSLQPEDFATYYCQQANHLPFTFGGGTKVEIK

SEQ ID NO: 15 - protein, 123 aa, synthetic construct
QLQLQESGPGLVKPSETLTLTCTVSADSISSTTYYWVWIRQPPGKGLEWIGSISYSGSTYYNPSLKSRVTVSVDTSKNQFSLKLNSVAATDTALYYCARHLGYNGRYLPFDYWGQGTLVTVSS

SEQ ID NO: 16 - protein, 110 aa, synthetic construct
QSALTQPASVSGSPGQSITISCTGTSSDVGFYNYVSWYQQHPGKAPELMIYDVSNRPSGVSDRFSGSKSGNTASLTISGLQAEDEADYYCSSYTSISTWVFGGGTKLTVL

SEQ ID NO: 17 - protein, 118 aa, synthetic construct
EVQLVESGGGLVQPGGSLRLSCAASGFAFSSYDMSWVRQAPGKGLDWVATISGGGRYTYYPDSVKGRFTISRDNSKNNLYLQMNSLRAEDTALYYCANRYGEAWFAYWGQGTLVTVSS

SEQ ID NO: 18 - protein, 107 aa, synthetic construct
DIQMTQSPSSMSASVGDRVTFTCRASQDINTYLSWFQQKPGKSPKTLIYRANRLVSGVPSRFSGSGSGQDYTLTISSLQPEDMATYYCLQYDEFPLTFGAGTKLELK

SEQ ID NO: 19 - protein, 118 aa, synthetic construct
EVQLVQSGGGLVQPGGSLKLSCAASGFTFSSYGMSWVRQAPGKGLDWVATISGGGRDTYYPDSVKGRFTISRDNSKNNLYLQMNSLRAEDTALYYCARQKGEAWFAYWGQGTLVTVSS

SEQ ID NO: 20 - protein, 111 aa, synthetic construct
DIVLTQSPASLAVSPGQRATITCRASESVDNYGISFMNWFQQKPGQPPKLLIYAASNKGTGVPARFSGSGSGTDFTLNINPMEENDTAMYFCQQSKEVPWTFGGGTKLEIK

SEQ ID NO: 21 - protein, 116 aa, synthetic construct
QVQLVESGGGLVKPGGSLRLSCAASGFTFSNYGMSWIRQAPGKGLEWVSTISGGGSNIYYADSVKGRFTISRDNAKNSLYLQMNSLRAEDTAVYYCVSYYYGIDFWGQGTSVTVSS

SEQ ID NO: 22 - protein, 107 aa, synthetic construct
DIQMTQSPSSLSASVGDRVTITCKASQDVTTAVAWYQQKPGKAPKLLIYWASTRHTGVPSRFSGSGSGTDFTLTISSLQPEDFATYYCQQHYTIPWTFGGGTKLEIK

SEQ ID NO: 23 - protein, 114 aa, synthetic construct
QIQLVQSGSELKKPGASVKVSCKASGYTFTNFGMNWVRQAPGQGLKWMGWISGYTREPTYAADFKGRFVISLDTSVSTAYLQISSLKAEDTAVYYCARDVFDYWGQGTLVTVSS

SEQ ID NO: 24 - protein, 110 aa, synthetic construct
DIVLTQSPASLAVSPGQRATITCRASESVDNYGYSFMNWFQQKPGQPPKLLIYRASNLESGVPARFSGSGSRTDFTLTINPVEADDTANYYCQQSNADPTFGQGTKLEIK

SEQ ID NO: 25 - protein, 123 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 26 - protein, 106 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 27 - protein, 118 aa, synthetic construct [sequence omitted]

SEQ ID NO: 28 - protein, 107 aa, synthetic construct [sequence omitted]

SEQ ID NO: 29 - protein, 121 aa, synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 30 - protein, 108 aa, synthetic construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 31 - protein, 119 aa, synthetic construct
QVQLVQSGAEVKKPGASVKLSCKASGYIFTTYWMHWVKQRPGQGLEWIGMIQPNSGGTKYNEKFKKKATLTVDKSISTAYMELSRLTSDDTAVYYCARGAGTVDYFDYWGQGSTLTVSS

SEQ ID NO: 32 - protein, 111 aa, synthetic construct
DIVLTQSPASLAVSVGQRATITCRASESVDIYGNSFMHWYQQKPGQPPKLLIYRASNLESGIPARFSGSGSRTDFTLTINPVEAQDTATYYCQQSTEDPYTFGGGTKLEIK

SEQ ID NO: 33 - protein, 121 aa, synthetic construct
EVQLLESGGGLVQPGGSLRLSCAASGFTFSSYAMSWVRQAPGKGLEWVSGISGSGGFTYYADSVKGRFTISRDNSKNTLYLQMNSLRAEDTAVYYCAKPPRGYNYGPFDYWGQGTLVTVSS

SEQ ID NO: 34 - protein, 108 aa, synthetic construct
SYVLTQPPSVSVAPGQTARITCGGNNIGSKSVHWYQQKPGQAPVLVVYDDSDRPSGIPERFSGSNSGNTATLTISRVEAGDEADYYCQVWDSSSDHVVFGGGTKLTVL

SEQ ID NO: 35 - protein, 119 aa, synthetic construct
EVQLVESGAEVKKPGSSVKVSCKASGGTFSSYAISWVRQAPGQGLEWMGGIIPIFGTANYAQKFQGRVTITADESTSTAYMELSSLRSEDTAVYYCARAPYYYYYMDVWGQGTTVTVSS

SEQ ID NO: 36 - protein, 109 aa, synthetic construct
QSALTQPASVSGSLGQSVTISCTGSSSDVGSYNLVSWYQQHPGKAPNLMIYDVSKRSGVSNRFSGSKSGNTASLTISGLQAEDEADYYCSSYTGISTVVFGGGTKLTVL

SEQ ID NO: 37 - protein, 120 aa, synthetic construct
QVQLQESGPGLVKPSETLSITCTVSGFSLSNYDISWIRQPPGKGLEWLGVIWTGGATNYNPALKSRLTISRDNSKNQVSLKMSSVTAADTAVYYCVRDSNYRYDEPFTYWGQGTLVTVSS

SEQ ID NO: 38 - protein, 107 aa, synthetic construct
EIVLTQSPDTLSVTPKEKVTLTCRASQSIGTNIHWFQQKPGQSPKLLIKYASESISGVPSRFSGSGSGTDFTLTINSVEAEDAATYYCQQSNSWPYTFGQGTKLEIK

SEQ ID NO: 39 - protein, 19 aa, Homo sapiens
VPSTPPTPSPSTPPTPSPS

SEQ ID NO: 40 - protein, 6 aa, Homo sapiens
VPPPPP

SEQ ID NO: 41 - protein, 58 aa, Homo sapiens
ESPKAQASSVPTAQPQAEGSLAKATTAPATTRNTGRGGEEKKKEKEKEEQEERETKTP

SEQ ID NO: 42 - protein, 15 aa, Homo sapiens
EPKSCDKTHTCPPCP

SEQ ID NO: 43 - protein, 11 aa, synthetic construct
SDKTHTCPPCP

SEQ ID NO: 44 - protein, 12 aa, Homo sapiens
ERKCCVECPPCP

SEQ ID NO: 45 - protein, 62 aa, Homo sapiens
ELKTPLGDTTHTCPRCPEPKSCDTPPPCPRCPEPKSCDTPPPCPRCPEPKSCDTPPPCPRCP

SEQ ID NO: 46 - protein, 12 aa, Homo sapiens
ESKYGPPCPSCP

SEQ ID NO: 47 - protein, 101 aa, Homo sapiens
CCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKS

SEQ ID NO: 48 - protein, 131 aa, Homo sapiens
GNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 49 - protein, 101 aa, Homo sapiens
CCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKS

SEQ ID NO: 50 - protein, 131 aa, Homo sapiens; identical to SEQ ID NO: 48

SEQ ID NO: 51 - protein, 108 aa, Homo sapiens
ECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREP

SEQ ID NO: 52 - protein, 117 aa, Homo sapiens
AAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 53 - protein, 107 aa, Homo sapiens
VCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCA

SEQ ID NO: 54 - protein, 108 aa, Homo sapiens
DSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTS

SEQ ID NO: 55 - protein, 110 aa, Homo sapiens
GPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 56 - protein, 110 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 57 - protein, 110 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAK

SEQ ID NO: 58 - protein, 106 aa, Homo sapiens [sequence omitted]

SEQ ID NO: 59 - protein, 109 aa, Homo sapiens
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTK

SEQ ID NO: 60 - protein, 107 aa, Homo sapiens
GQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 61 - protein, 110 aa, Homo sapiens
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTK

SEQ ID NO: 62 - protein, 107 aa, Homo sapiens
GQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 63 - protein, 110 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 64 - protein, 107 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 65 - protein, 112 aa, Homo sapiens
VIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVP

SEQ ID NO: 66 - protein, 106 aa, Homo sapiens
DQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPK

SEQ ID NO: 67 - protein, 131 aa, Homo sapiens
GVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 68 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 69 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 70 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTA

SEQ ID NO: 71 - protein, 114 aa, synthetic construct
NWVNVISDLKKIEDLIQSMHIDATLYTESDVHPSCKVTAMKCFLLELQDISLESGDASIHDTVENLIILANNSLSSNGNVTESGCKECEELEEKNIKEFLQSFVHIVQMFINTA

SEQ ID NO: 72 - protein, 114 aa, synthetic construct
NWVNVISDLKKIEDLIQSMHIDATLYTESDVHPSCKVTAMKCFLLELQVDSLESGDASIHDTVENLIILANNSLSSNGNVTESGCKECEELEEKNIKEFLQSFVHIVQMFINTA

SEQ ID NO: 73 - protein, 114 aa, synthetic construct
NWVNVISDLKKIEDLIQSMHIDATLYTESDVHPSCKVTAMKCFLLELQVISLESGDASIHDTVENEIILANNSLSSNGNVTESGCKECEELEEKNIKEFLQSFVHIVQMFINTA

SEQ ID NO: 74 - protein, 114 aa, synthetic construct
NWVNVISNLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTA

SEQ ID NO: 75 - protein, 114 aa, synthetic construct
NWVNVISDLKKIEDLIQSMHIDATLYTESDVHPSCKVTAMKCFLLKLQVISLESGDASIHDTVENLIILANNSLSSNGNVTESGCKECEELEEKNIKEFLQSFVHIVQMFINTA

SEQ ID NO: 76 - protein, 114 aa, synthetic construct
NWVNVISDLKKIEDLIQSMHIDATLYTESDVHPSCKVTAMKCFLLKLQVISLKSGDASIHDTVENLIILANNSLSSNGNVTESGCKECEELEEKNIKEFLQSFVHIVQMFINTA

SEQ ID NO: 77 - protein, 114 aa, synthetic construct
NWVNVISDLKKIEDLIQSMHIKATLYTESDVHPSCKVTAMKCFLLKLQVISLESGDASIHDTVENLIILANNSLSSNGNVTESGCKECEELEEKNIKEFLQSFVHIVQMFINTA

SEQ ID NO: 78 - protein, 114 aa, synthetic construct
NWVNVISDLKKIEDLIQSMHIKATLYTESDVHPSCKVTAMKCFLLKLQVISLKSGDASIHDTVENLIILANNSLSSNGNVTESGCKECEELEEKNIKEFLQSFVHIVQMFINTA

SEQ ID NO: 79 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCGECEELEEKNIKEFLQSFVHIVQMFINTA

SEQ ID NO: 80 - protein, 267 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 81 - protein, 175 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT

SEQ ID NO: 82 - protein, 78 aa, Homo sapiens
ITCPPPMSVEHADIWVKSYSLYSRERYICNSGFKRKAGTSSLTECVLNKATNVAHWTTPSLKCIRDPALVHQRPAPPS

SEQ ID NO: 83 - protein, 65 aa, Homo sapiens
ITCPPPMSVEHADIWVKSYSLYSRERYICNSGFKRKAGTSSLTECVLNKATNVAHWTTPSLKCIR

SEQ ID NO: 84 - protein, 78 aa, synthetic construct
ITCPPPMSVEHADIWVKSYSLYSRERYICNSGFKEKAGTSSLTECVLNKATNVAHWTTPSLKCIRDPALVHQRPAPPS

SEQ ID NO: 85 - protein, 78 aa, synthetic construct
ITCPPPMSVEHADIWVKSYSLYSREEYICNSGFKEKAGTSSLTECVLNKATNVAHWTTPSLKCIRDPALVHQRPAPPS

SEQ ID NO: 86 - protein, 78 aa, synthetic construct
ITCPPPMSVEHADIWVKSYSLYSEEEYICNSGFKEKAGTSSLTECVLNKATNVAHWTTPSLKCIRDPALVHQRPAPPS

SEQ ID NO: 87 - protein, 78 aa, synthetic construct
IACPPPMSVEHADIWVKSYSLYSRERYICNSGFKRKAGTSSLTECVLNKATNVAHWTTPSLKCIRDPALVHQRPAPPS

SEQ ID NO: 88 - protein, 65 aa, synthetic construct
IACPPPMSVEHADIWVKSYSLYSRERYICNSGFKRKAGTSSLTECVLNKATNVAHWTTPSLKCIR

SEQ ID NO: 89 - protein, 12 aa, synthetic construct
PLGLSGRSDNQG

SEQ ID NO: 90 - protein, 26 aa, synthetic construct
GGGGSPLGLSGRSDNQGGGGSGGGGS

SEQ ID NO: 91 - protein, 15 aa, synthetic construct
PLGLAGSGRSDNQGA

SEQ ID NO: 92 - protein, 16 aa, synthetic construct
GPLGLAGSGRSDNQGA

SEQ ID NO: 93 - protein, 17 aa, synthetic construct
GGPLGLAGSGRSDNQGA

SEQ ID NO: 94 - protein, 19 aa, synthetic construct
GGSPLGLAGSGRSDNHGGA

SEQ ID NO: 95 - protein, 19 aa, synthetic construct
GGSPLGLAGSGRSDNQGGA

SEQ ID NO: 96 - protein, 19 aa, synthetic construct
GGSPLGLAGSGRSDNRGGA

SEQ ID NO: 97 - protein, 19 aa, synthetic construct
GGSPLGLAGSGRSDNTGGA

SEQ ID NO: 98 - protein, 13 aa, synthetic construct
GGGSGRSDNHGGA

SEQ ID NO: 99 - protein, 14 aa, synthetic construct
GGGGSGRSDNHGGA

SEQ ID NO: 100 - protein, 16 aa, synthetic construct
GGGGSSGRSDNHGGGA

SEQ ID NO: 101 - protein, 18 aa, synthetic construct
GGSGGSSGRSDNHGGGGA

SEQ ID NO: 102 - protein, 19 aa, synthetic construct
GGSGGSSGRSDNHGGSGGA

SEQ ID NO: 103 - protein, 12 aa, synthetic construct
GGSGRSDNHGGA

SEQ ID NO: 104 - protein, 15 aa, synthetic construct
GGSGGSGRSDNHGGA

SEQ ID NO: 105 - protein, 9 aa, synthetic construct
GSPLGLAGS

SEQ ID NO: 106 - protein, 11 aa, synthetic construct
GGSPLGLAGGS

SEQ ID NO: 107 - protein, 12 aa, synthetic construct
GGSPLGLAGGGS

SEQ ID NO: 108 - protein, 13 aa, synthetic construct
GGGSPLGLAGGGS

SEQ ID NO: 109 - protein, 14 aa, synthetic construct
GGGSPLGLAGGGGS

SEQ ID NO: 110 - protein, 15 aa, synthetic construct
GGSGGGPLGLAGGGA

SEQ ID NO: 111 - protein, 7 aa, synthetic construct
SGRSDNA

SEQ ID NO: 112 - protein, 7 aa, synthetic construct
SGRSDND

SEQ ID NO: 113 - protein, 7 aa, synthetic construct
SGRSDNE

SEQ ID NO: 114 - protein, 7 aa, synthetic construct
SGRSDNF

SEQ ID NO: 115 - protein, 7 aa, synthetic construct
SGRSDNG

SEQ ID NO: 116 - protein, 7 aa, synthetic construct
SGRSDNI

SEQ ID NO: 117 - protein, 7 aa, synthetic construct
SGRSDNK

SEQ ID NO: 118 - protein, 7 aa, synthetic construct
SGRSDNL

SEQ ID NO: 119 - protein, 7 aa, synthetic construct
SGRSDNM

SEQ ID NO: 120 - protein, 7 aa, synthetic construct
SGRSDNN

SEQ ID NO: 121 - protein, 7 aa, synthetic construct
SGRSDNP

SEQ ID NO: 122 - protein, 7 aa, synthetic construct
SGRSDNQ

SEQ ID NO: 123 - protein, 7 aa, synthetic construct
SGRSDNR

SEQ ID NO: 124 - protein, 7 aa, synthetic construct
SGRSDNS

SEQ ID NO: 125 - protein, 7 aa, synthetic construct
SGRSDNT

SEQ ID NO: 126 - protein, 7 aa, synthetic construct
SGRSDNV

SEQ ID NO: 127 - protein, 7 aa, synthetic construct
SGRSDNW

SEQ ID NO: 128 - protein, 7 aa, synthetic construct
SGRSDNY

SEQ ID NO: 129 - protein, 13 aa, synthetic construct
PLGLAGSGRSDNR

SEQ ID NO: 130 - protein, 13 aa, synthetic construct
PLGLAGSGRSDNQ

SEQ ID NO: 131 - protein, 13 aa, synthetic construct
PLGLAGSGRSDNT

SEQ ID NO: 132 - protein, 13 aa, synthetic construct
PLGLAGSGRSDNN

SEQ ID NO: 133 - protein, 13 aa, synthetic construct
PLGLAGSGRSDNV

SEQ ID NO: 134 - protein, 13 aa, synthetic construct
PLGLAGSGRSDNW

SEQ ID NO: 135 - protein, 13 aa, synthetic construct
PLGLAGSGRSDNY

SEQ ID NO: 136 - protein, 677 aa, synthetic construct
QVQLVQSGSELKKPGASVKVSCKASGYTFTNYDINWVRQAPGQGLEWMGWIFPGDESTQYNEKFKGRFVFSLDTSVSTAYLQISSLKAEDTAVYYCARQTTATWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGPLGLSGRSDNQGNWVNVISDLKKIEDLIQSMHIDATLYTESDVHPSCKVTAMKCFLLELQVISLESGDASIHDTVENLIILANNSLSSNGNVTESGCGECEELEEKNIKEFLQSFVHIVQMFINTAGGGGSPLGLSGRSDNQGGGGSGGGGSIACPPPMSVEHADIWVKSYSLYSRERYICNSGFKRKAGTSSLTECVLNKATNVAHWTTPSLKCIRDPALVHQRPAPPS

SEQ ID NO: 137 - protein, 214 aa, synthetic construct
DVVMTQSPAFLSVTPGEKVTITCRASQSISDYLHWYQQKPDQAPKLLIKYASQSISGIPARFSGSGSGTDFTFTISSLEAEDAATYYCQNGHSFPLTFGQGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 138 - protein, 677 aa, synthetic construct
QVQLVQSGSELKKPGASVKVSCKASGYTFTNYDINWVRQAPGQGLEWMGWIFPGDESTQYNEKFKGRFVFSLDTSVSTAYLQISSLKAEDTAVYYCARQTTATWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGGSGGSGGSNWVNVISDLKKIEDLIQSMHIDATLYTESDVHPSCKVTAMKCFLLELQVISLESGDASIHDTVENLIILANNSLSSNGNVTESGCGECEELEEKNIKEFLQSFVHIVQMFINTAGGGGSGGGGSGGSGGSGGGGSGGGGSIACPPPMSVEHADIWVKSYSLYSRERYICNSGFKRKAGTSSLTECVLNKATNVAHWTTPSLKCIRDPALVHQRPAPPS

SEQ ID NO: 139 - protein, 214 aa, synthetic construct; identical to SEQ ID NO: 137

SEQ ID NO: 140 - protein, 453 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - protein, 213 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 142 - protein, 443 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 144 - protein, 678 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGSNWVNVISDLKKIEDLIQSMHIDATLYTESDVHPSCKVTAMKCFLLELQVISLESGDASIHDTVENLIILANNSLSSNGNVTESGCGECEELEEKNIKEFLQSFVHIVQMFINTAGGGGSPLGLSGRSDNQGGGGSGGGGSIACPPPMSVEHADIWVKSYSLYSRERYICNSGFKRKAGTSSLTECVLNKATNVAHWTTPSLKCIRDPALVHQRPAPPS

SEQ ID NO: 145 - protein, 213 aa, synthetic construct; identical to SEQ ID NO: 141

SEQ ID NO: 146 - protein, 668 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGSNWVNVISDLKKIEDLIQSMHIDATLYTESDVHPSCKVTAMKCFLLELQVISLESGDASIHDTVENLIILANNSLSSNGNVTESGCGECEELEEKNIKEFLQSFVHIVQMFINTAGGGGSPLGLSGRSDNQGGGGSGGGGSIACPPPMSVEHADIWVKSYSLYSRERYICNSGFKRKAGTSSLTECVLNKATNVAHWTTPSLKCIRDPALVHQRPAPPS

SEQ ID NO: 147 - protein, 214 aa, synthetic construct; identical to SEQ ID NO: 143

SEQ ID NO: 148 - protein, 657 aa, synthetic construct
QVQLVQSGAEVKKPGSSVKVSCKASGFTFTTYYISWVRQAPGQGLEYLGYINMGSGGTNYNEKFKGRVTITADKSTSTAYMELSSLRSEDTAVYYCAIIGYFDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGSNWVNVISDLKKIEDLIQSMHIDATLYTESDVHPSCKVTAMKCFLLELQVISLESGDASIHDTVENLIILANNSLSSNGNVTESGCGECEELEEKNIKEFLQSFVHIVQMFINTAGGGGSPLGLSGRSDNQGGGGSGGGGSIACPPPMSVEHADIWVKSYSLYSRERYICNSGFKRKAGTSSLTECVLNKATNVAHWTTPSLKCIR

SEQ ID NO: 149 - protein, 219 aa, synthetic construct
DVVMTQSPLSLPVTLGQPASISCRSSQSLLDSDGGTYLYWFQQRPGQSPRRLIYLVSTLGSGVPDRFSGSGSGTDFTLKISRVEAEDVGVYYCMQLTHWPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 150 - protein, 4 aa, synthetic construct
  REGION 1..4: note = thrombin cleavable amino acid sequence
GRGD

SEQ ID NO: 151 - protein, 6 aa, synthetic construct
  REGION 1..6: note = thrombin cleavable amino acid sequence
GRGDNP

SEQ ID NO: 152 - protein, 5 aa, synthetic construct
  REGION 1..5: note = thrombin cleavable amino acid sequence
GRGDS

SEQ ID NO: 153 - protein, 8 aa, synthetic construct
  REGION 1..8: note = thrombin cleavable amino acid sequence
GRGSDSPK

SEQ ID NO: 154 - protein, 4 aa, synthetic construct
  REGION 1..4: note = elastase cleavable amino acid sequence
AAPV

SEQ ID NO: 155 - protein, 4 aa, synthetic construct
  REGION 1..4: note = elastase cleavable amino acid sequence
AAPL

SEQ ID NO: 156 - protein, 4 aa, synthetic construct
  REGION 1..4: note = elastase cleavable amino acid sequence
AAPF

SEQ ID NO: 157 - protein, 4 aa, synthetic construct
  REGION 1..4: note = elastase cleavable amino acid sequence
AAPA

SEQ ID NO: 158 - protein, 4 aa, synthetic construct
  REGION 1..4: note = elastase cleavable amino acid sequence
AYLV

SEQ ID NO: 159 - protein, 6 aa, synthetic construct
  REGION 1..6: note = matrix metalloproteinase cleavable amino acid sequence
  VARIANT 3: note = Xaa is any amino acid
  VARIANT 6: note = Xaa is any amino acid
GPXGPX

SEQ ID NO: 160 - skipped

SEQ ID NO: 161 - protein, 6 aa, synthetic construct
  REGION 1..6: note = matrix metalloproteinase cleavable amino acid sequence
  VARIANT 6: note = Xaa is any amino acid
GPIGPX

SEQ ID NO: 162 - protein, 5 aa, synthetic construct
  REGION 1..5: note = matrix metalloproteinase cleavable amino acid sequence
  VARIANT 5: note = Xaa is any amino acid
APGLX

SEQ ID NO: 163 - protein, 7 aa, synthetic construct
  REGION 1..7: note = collagenase cleavable amino acid sequence
  VARIANT 7: note = Xaa is any amino acid
PLGPDRX

SEQ ID NO: 164 - protein, 7 aa, synthetic construct
  REGION 1..7: note = collagenase cleavable amino acid sequence
  VARIANT 7: note = Xaa is any amino acid
PLGLLGX

SEQ ID NO: 165 - protein, 7 aa, synthetic construct
  REGION 1..7: note = collagenase cleavable amino acid sequence
PQGIAGW

SEQ ID NO: 166 - protein, 5 aa, synthetic construct
  REGION 1..5: note = collagenase cleavable amino acid sequence
PLGCH

SEQ ID NO: 167 - protein, 6 aa, synthetic construct
  REGION 1..6: note = collagenase cleavable amino acid sequence
PLGLYA

SEQ ID NO: 168 - protein, 7 aa, synthetic construct
  REGION 1..7: note = collagenase cleavable amino acid sequence
PLALWAR

SEQ ID NO: 169 - protein, 7 aa, synthetic construct
  REGION 1..7: note = collagenase cleavable amino acid sequence
PLAYWAR

SEQ ID NO: 170 - protein, 7 aa, synthetic construct
  REGION 1..7: note = stromelysin cleavable amino acid sequence
PYAYYMR

SEQ ID NO: 171 - protein, 7 aa, synthetic construct
  REGION 1..7: note = stromelysin cleavable amino acid sequence
PLGMYSR

SEQ ID NO: 172 - protein, 4 aa, synthetic construct
  REGION 1..4: note = angiotensin converting enzyme cleavable amino acid sequence
GDKP

SEQ ID NO: 173 - protein, 5 aa, synthetic construct
  REGION 1..5: note = angiotensin converting enzyme cleavable amino acid sequence
GSDKP

SEQ ID NO: 174 - protein, 4 aa, synthetic construct
  REGION 1..4: note = cathepsin B cleavable amino acid sequence
ALAL

SEQ ID NO: 175 - protein, 4 aa, synthetic construct
  REGION 1..4: note = cathepsin B cleavable amino acid sequence
GFLG

SEQ ID NO: 176 - protein, 4 aa, synthetic construct
  REGION 1..4: note = non-cleavable linker sequence
GSGS

SEQ ID NO: 177 - protein, 4 aa, synthetic construct
  REGION 1..4: note = non-cleavable linker sequence
GGSG

SEQ ID NO: 178 - protein, 4 aa, synthetic construct
  REGION 1..4: note = non-cleavable linker sequence
GGGS

SEQ ID NO: 179 - protein, 5 aa, synthetic construct
  REGION 1..5: note = non-cleavable linker sequence
GGGGS

SEQ ID NO: 180 - protein, 4 aa, synthetic construct
  REGION 1..4: note = non-cleavable linker sequence
GNGN

SEQ ID NO: 181 - protein, 4 aa, synthetic construct
  REGION 1..4: note = non-cleavable linker sequence
GGNG

SEQ ID NO: 182 - protein, 4 aa, synthetic construct
  REGION 1..4: note = non-cleavable linker sequence
GGGN

SEQ ID NO: 183 - protein, 5 aa, synthetic construct
  REGION 1..5: note = non-cleavable linker sequence
GGGGN

SEQ ID NO: 184 - protein, 15 aa, synthetic construct
  REGION 1..15: note = non-cleavable linker sequence
GGGGSGGGGSGGGGS

SEQ ID NO: 185 - protein, 22 aa, synthetic construct
  REGION 1..22: note = non-cleavable linker sequence
GSGGGGSGGGGSGGGGSGGGGS

SEQ ID NO: 186 - protein, 30 aa, synthetic construct
  REGION 1..30: note = non-cleavable linker sequence
GGGGSGGGGSGGGGSGGGGSGGGGSGGGGS

SEQ ID NO: 187 - protein, 25 aa, synthetic construct
  REGION 1..25: note = non-cleavable linker sequence
DAAAKEAAAKSAAAREAAARDAAAK

SEQ ID NO: 188 - protein, 14 aa, synthetic construct
  REGION 1..14: note = non-cleavable linker sequence
NVDHKPSNTKVDKR

SEQ ID NO: 189 - protein, 5 aa, synthetic construct
  REGION 1..5: note = non-cleavable linker sequence
DGGGS

SEQ ID NO: 190 - protein, 5 aa, synthetic construct
  REGION 1..5: note = non-cleavable linker sequence
TGEKP

SEQ ID NO: 191 - protein, 4 aa, synthetic construct
  REGION 1..4: note = non-cleavable linker sequence
GGRR

SEQ ID NO: 192 - protein, 5 aa, synthetic construct
GGGGS

SEQ ID NO: 193 - protein, 14 aa, synthetic construct
  REGION 1..14: note = non-cleavable linker sequence
EGKSSGSGSESKVD

SEQ ID NO: 194 - protein, 18 aa, synthetic construct
  REGION 1..18: note = non-cleavable linker sequence
KESGSVSSEQLAQFRSLD

SEQ ID NO: 195 - protein, 8 aa, synthetic construct
  REGION 1..8: note = non-cleavable linker sequence
GGRRGGGS

SEQ ID NO: 196 - protein, 9 aa, synthetic construct
  REGION 1..9: note = non-cleavable linker sequence
LRQRDGERP

SEQ ID NO: 197 - protein, 12 aa, synthetic construct
  REGION 1..12: note = non-cleavable linker sequence
LRQKDGGGSERP

SEQ ID NO: 198 - protein, 16 aa, synthetic construct
  REGION 1..16: note = non-cleavable linker sequence
LRQKDGGGSGGGSERP

SEQ ID NO: 199 - protein, 668 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGSNWVNVISDLKKIEDLIQSMHIDATLYTESDVHPSCKVTAMKCFLLELQVISLESGDASIHDTVENLIILANNSLSSNGNVTESGCGECEELEEKNIKEFLQSFVHIVQMFINTAGGGSPLGLAGSGRSDNQGGSGGSGGSIACPPPMSVEHADIWVKSYSLYSRERYICNSGFKRKAGTSSLTECVLNKATNVAHWTTPSLKCIRDPALVHQRPAPPS

SEQ ID NO: 200 - protein, 214 aa, synthetic construct; identical to SEQ ID NO: 143

SEQ ID NO: 201 - protein, 26 aa, synthetic construct
GGGSPLGLAGSGRSDNQGGSGGSGGS

SEQ ID NO: 202 - protein, 118 aa, synthetic construct
QVQLVQSGSELKKPGASVKVSCKASGYTFTNYDINWVRQAPGQGLEWMGWIFPGDESTQYNEKFKGRFVFSLDTSVSTAYLQISSLKAEDTAVYYCARQTTATWFAYWGQGTLVTVSS

SEQ ID NO: 203 - protein, 107 aa, synthetic construct
DVVMTQSPAFLSVTPGEKVTITCRASQSISDYLHWYQQKPDQAPKLLIKYASQSISGIPARFSGSGSGTDFTFTISSLEAEDAATYYCQNGHSFPLTFGQGTKLELK

(19) United States
(12) Patent Application Publication
(10) Pub. No.: US 2024/0182537 A1
(43) Pub. Date: Jun. 6, 2024

(54) IL-15 PROCYTOKINE ANTIBODY FUSION PROTEINS

(71) Applicant: Proviva Therapeutics (Hong Kong) Limited, Hong Kong (CN)

(72) Inventors: Zijuan LI, Shanghai (CN); Feifei ZHANG, Shanghai (CN); Ze ZHANG, Shanghai (CN); Hongxing ZHOU, Bedford, MA (US)

(21) Appl. No.: 18/459,142

(22) Filed: Aug. 31, 2023

Related U.S. Application Data
(60) Provisional application No. 63/402,639, filed on Aug. 31, 2022.

(51) Int. Cl. C07K 14/54; A61K 38/00; A61P 35/00; C07K 14/715; C07K 16/28

(52) U.S. Cl. CPC C07K 14/5443; A61P 35/00; C07K 14/7155; C07K 16/2818; A61K 38/00; C07K 2317/524; C07K 2317/526; C07K 2317/55; C07K 2317/565; C07K 2319/30

(57) ABSTRACT

Provided are activatable proprotein homodimers comprising two separate but identical polypeptide chains, each chain comprising a fragment antigen-binding (Fab) region that specifically binds to human PD-1 or human PD-L1 or human B7H3, a hinge/Fc domain, a linker, an IL-15 protein, a protease cleavable linker, and an IL-15Rα protein. Also provided are related pharmaceutical compositions and methods of use thereof.

### CROSS-REFERENCE TO RELATED APPLICATIONS

This application claims the benefit under 35 U.S.C. § 119(e) of U.S. Provisional Application No. 63/402,639, filed Aug. 31, 2022, which is incorporated by reference in its entirety.

### STATEMENT REGARDING THE SEQUENCE LISTING

The Sequence Listing XML associated with this application is provided in XML file format and is hereby incorporated by reference into the specification. The name of the XML file containing the Sequence Listing XML is PRVA_016_01US_ST26.xml. The XML file is about 212,188 bytes, was created on Aug. 28, 2023, and is being submitted electronically via USPTO Patent Center.

### BACKGROUND

### Technical Field

The present disclosure relates to activatable proprotein homodimers comprising two separate but identical polypeptide chains, each chain comprising a fragment antigen-binding (Fab) region that specifically binds to human PD-1 or human PD-L1 or human B7H3, a hinge/Fc domain, a linker, an IL-15 protein, a protease cleavable linker, and an IL-15Rα protein. Also included are related pharmaceutical compositions and methods of use thereof.

### Description of the Related Art

Interleukin-15 (IL-15) immunotherapy has proven utility in the treatment of cancers such as malignant melanoma and renal cell cancer, among others. Programmed cell death protein 1 (PD-1) and programmed death ligand 1 (PD-L1) inhibitor therapy enhances anti-tumor T-cell response and mediates antitumor activity (Dermani et al., J Cell Physiol. 234:1313-1325, 2019).

However, there are certain problems associated with most IL-15 therapies. For example, IL-15 has been shown to exhibit a short half-life and high doses can be required to achieve biological responses in vivo, resulting in clinical toxicities and limited anti-tumor responses in patients. IL-15 and IL-15 derivatives are under development to increase therapeutic effectiveness. However, significant drawbacks exist, including high serum Cmax initially causing over-activation of immune system, short PK due to either small molecular size for IL-15 (13-14 kD) or catabolism by the large number of immune cells expressing IL-15 receptors for IL-15 or IL-15 Fc fusion proteins, poor accumulation in the target tumor due to short PK, lack of or ineffective tumor targeting, and undesirable accumulation and immune activation activities in normal tissues. Nonetheless, IL-15 therapies can be effective, and there are strategies for addressing these and other drawbacks (see, for example, WO 2020/123980). However, there is still a need in the art to improve on such strategies.

Embodiments of the present disclosure represent such improvements by providing anti-PD-1/PD-L1 activatable proproteins comprising IL-15 that can be specifically targeted to, and activated within, the tumor microenvironment (TME).

### BRIEF SUMMARY

Embodiments of the present disclosure include an activatable proprotein homodimer, comprising a first polypeptide and a second polypeptide, wherein the first polypeptide and the second polypeptide comprise, in an N- to C-terminal orientation, a fragment antigen-binding (Fab) region that specifically binds to human PD-1 or human PD-L1 or human B7H3, a hinge/Fc domain, a first linker, an IL-15 protein, a second linker, and an IL-15Rα protein, wherein the hinge/Fc domain of the first polypeptide binds to the hinge/Fc domain of the second polypeptide, wherein the IL-15 protein of the first polypeptide binds to the IL-15Rα protein of the second polypeptide, and wherein the IL-15Rα of the first polypeptide binds to the IL-15 protein of the second polypeptide, wherein said binding masks a binding site of the IL-15 protein(s) that otherwise binds to an IL-15β/γc chain present on the surface of an immune cell in vitro or in vivo, and wherein the second linker is a cleavable linker.

In some embodiments, the Fab region specifically binds to human PD-1, and optionally comprises the Fab region from an anti-PD-1 antibody selected from nivolumab, pembrolizumab, cemiplimab, JTX-4014, spartalizumab, camrelizumab, sintilimab, tislelizumab, toripalimab, dostarlimab, MGA012, AMP-22, and AMP-514. In some embodiments, the Fab region specifically binds to human PD-1 and comprises
a heavy chain variable (VH) region comprising VHCDR1, VHCDR2, and VHCDR3 regions set forth in SEQ ID NO: 1; and a light chain variable (VL) region comprising VLCDR1, VLCDR2, and VLCDR3 regions set forth in SEQ ID NO: 2;
a heavy chain variable (VH) region comprising VHCDR1, VHCDR2, and VHCDR3 regions set forth in SEQ ID NO: 3; and a light chain variable (VL) region comprising VLCDR1, VLCDR2, and VLCDR3 regions set forth in SEQ ID NO: 4;
a heavy chain variable (VH) region comprising VHCDR1, VHCDR2, and VHCDR3 regions set forth in SEQ ID NO: 5; and a light chain variable (VL) region comprising VLCDR1, VLCDR2, and VLCDR3 regions set forth in SEQ ID NO: 6; or
a heavy chain variable (VH) region comprising VHCDR1, VHCDR2, and VHCDR3 regions set forth in SEQ ID NO: 7; and a light chain variable (VL) region comprising VLCDR1, VLCDR2, and VLCDR3 regions set forth in SEQ ID NO: 8;
a heavy chain variable (VH) region comprising VHCDR1, VHCDR2, and VHCDR3 regions set forth in SEQ ID NO: 9; and a light chain variable (VL) region comprising VLCDR1, VLCDR2, and VLCDR3 regions set forth in SEQ ID NO: 10;
a heavy chain variable (VH) region comprising VHCDR1, VHCDR2, and VHCDR3 regions set forth in SEQ ID NO: 11; and a light chain variable (VL) region comprising VLCDR1, VLCDR2, and VLCDR3 regions set forth in SEQ ID NO: 12;
a heavy chain variable (VH) region comprising VHCDR1, VHCDR2, and VHCDR3 regions set forth in SEQ ID NO: 13; and a light chain variable (VL) region comprising VLCDR1, VLCDR2, and VLCDR3 regions set forth in SEQ ID NO: 14;
a heavy chain variable (VH) region comprising VHCDR1, VHCDR2, and VHCDR3 regions set forth in SEQ ID NO: 15; and a light chain variable (VL) region comprising VLCDR1, VLCDR2, and VLCDR3 regions set forth in SEQ ID NO: 16;
a heavy chain variable (VH) region comprising VHCDR1, VHCDR2, and VHCDR3 regions set forth in SEQ ID NO: 17; and a light chain variable (VL) region comprising VLCDR1, VLCDR2, and VLCDR3 regions set forth in SEQ ID NO: 18;
a heavy chain variable (VH) region comprising VHCDR1, VHCDR2, and VHCDR3 regions set forth in SEQ ID NO: 19; and a light chain variable (VL) region comprising VLCDR1, VLCDR2, and VLCDR3 regions set forth in SEQ ID NO: 20;
a heavy chain variable (VH) region comprising VHCDR1, VHCDR2, and VHCDR3 regions set forth in SEQ ID NO: 21; and a light chain variable (VL) region comprising VLCDR1, VLCDR2, and VLCDR3 regions set forth in SEQ ID NO: 22; or a heavy chain variable (VH) region comprising VHCDR1, VHCDR2, and VHCDR3 regions set forth in SEQ ID NO: 23; and a light chain variable (VL) region comprising VLCDR1, VLCDR2, and VLCDR3 regions set forth in SEQ ID NO: 24.

In some embodiments, the VH region comprises an amino acid sequence having at least 90%, 95%, 96%, 97%, 98%, 99%, or 100% identity to a sequence selected from SEQ ID NOs: 1, 3, 5, 7, 9, 11, 13, 14, 15, 17, 19, 21, and 23, and the VL region respectively comprises an amino acid sequence having at least 90%, 95%, 96%, 97%, 98%, 99%, or 100% identity to a sequence selected from SEQ ID NOs: 2, 4, 6, 8, 10, 12, 14, 16, 18, 20, 22, and 24.

In some embodiments, the Fab region specifically binds to human PD-L1, and optionally comprises the Fab region from an anti-PD-L1 antibody selected from atezolizumab, avelumab, and durvalumab. In some embodiments, the Fab region specifically binds to human PD-L1 and comprises a heavy chain variable (VH) region comprising VHCDR1, VHCDR2, and VHCDR3 regions set forth in SEQ ID NO: 25; and a light chain variable (VL) region comprising VLCDR1, VLCDR2, and VLCDR3 regions set forth in SEQ ID NO: 26;
a heavy chain variable (VH) region comprising VHCDR1, VHCDR2, and VHCDR3 regions set forth in SEQ ID NO: 27; and a light chain variable (VL) region comprising VLCDR1, VLCDR2, and VLCDR3 regions set forth in SEQ ID NO: 28;
a heavy chain variable (VH) region comprising VHCDR1, VHCDR2, and VHCDR3 regions set forth in SEQ ID NO: 29; and a light chain variable (VL) region comprising VLCDR1, VLCDR2, and VLCDR3 regions set forth in SEQ ID NO: 30; or
a heavy chain variable (VH) region comprising VHCDR1, VHCDR2, and VHCDR3 regions set forth in SEQ ID NO: 31; and a light chain variable (VL) region comprising VLCDR1, VLCDR2, and VLCDR3 regions set forth in SEQ ID NO: 32;
a heavy chain variable (VH) region comprising VHCDR1, VHCDR2, and VHCDR3 regions set forth in SEQ ID NO: 33; and a light chain variable (VL) region comprising VLCDR1, VLCDR2, and VLCDR3 regions set forth in SEQ ID NO: 34;
a heavy chain variable (VH) region comprising VHCDR1, VHCDR2, and VHCDR3 regions set forth in SEQ ID NO: 35; and a light chain variable (VL) region comprising VLCDR1, VLCDR2, and VLCDR3 regions set forth in SEQ ID NO: 36; or
a heavy chain variable (VH) region comprising VHCDR1, VHCDR2, and VHCDR3 regions set forth in SEQ ID NO: 37; and a light chain variable (VL) region comprising VLCDR1, VLCDR2, and VLCDR3 regions set forth in SEQ ID NO: 38.

In some embodiments, the VH region comprises an amino acid sequence having at least 90%, 95%, 96%, 97%, 98%, 99%, or 100% identity to a sequence selected from SEQ ID NOs: 25, 27, 29, 31, 33, 35, and 37, and the VL region respectively comprises an amino acid sequence having at least 90%, 95%, 96%, 97%, 98%, 99%, or 100% identity to a sequence selected from SEQ ID NOs: 26, 28, 30, 32, 34, 36, and 38.

In some embodiments, the Fab region specifically binds to human B7H3 and comprises a heavy chain variable (VH) region comprising VHCDR1, VHCDR2, and VHCDR3 regions set forth in SEQ ID NO: 202; and a light chain variable (VL) region comprising VLCDR1, VLCDR2, and VLCDR3 regions set forth in SEQ ID NO: 203. In specific embodiments, the VH region comprises an amino acid sequence having at least 90%, 95%, 96%, 97%, 98%, 99%, or 100% identity to SEQ ID NO: 202, and the VL region respectively comprises an amino acid sequence having at least 90%, 95%, 96%, 97%, 98%, 99%, or 100% identity to SEQ ID NO: 203.

In some embodiments, the Fc domain comprises a CH2 domain, a CH3 domain, or a CH2CH3 domain of an immunoglobulin, optionally wherein the immunoglobulin is from an immunoglobulin class selected from IgG1, IgG2, IgG3, IgG4, IgA, IgD, IgE, and IgM. In some embodiments, the hinge comprises an amino acid sequence having at least 90%, 95%, 96%, 97%, 98%, 99%, or 100% identity to a sequence selected from Table F1, and wherein the Fc domain comprises an amino acid sequence having at least 90%, 95%, 96%, 97%, 98%, 99%, or 100% identity to a sequence selected from Table F1. In some embodiments, the Fc domain is a modified Fc domain that does not bind or substantially bind to FcγR, and retains normal or substantially normal binding to FcRn. In some embodiments, the modified Fc domain comprises a modified IgG1 CH2 domain with the L234A/L235A (“LALA”) mutations and/or the P329A or P329G mutation (EU numbering).

In some embodiments, the IL-15 protein comprises, consists, or consists essentially of an amino acid sequence that is at least 80, 85, 90, 95, 98, or 100% identical to a sequence selected from Table S1, optionally wherein the IL-15 protein comprises, consists, or consists essentially of an amino acid sequence that is at least 80, 85, 90, 95, 98, or 100% identical to SEQ ID NO: 79 which retains the K86G and S162A mutations. In some embodiments, the IL-15 protein comprises or retains one or more amino acid substitutions at position D8, D22, E46, V49, I50, L66, and/or K86 as defined by SEQ ID NO: 69 (mature human IL-15), and/or S162 as defined by SEQ ID NO: 68 (IL-15 FL precursor). In some embodiments, the one or more amino acid substitutions are selected from D8N, D22K, E46K, V49D, 150D, L66E, K86G, and 162A, optionally the combination of K86G and S162A.

In some embodiments, the one or more amino acid substitutions are combinations selected from K86G and 162A, V49D and S162A; I50D and S162A; L66E and S162A; D8N and S162A; V49D and S162A; E46K and S162A; E46K, E53K, and S162A; D22K, E46K, and S162A; and D22K, E46K, E53K, and S162A.

In some embodiments, the IL-15Rα protein comprises, consists, or consists essentially of an amino acid sequence that is at least 80, 85, 90, 95, 98, or 100% identical to a sequence selected from Table S2, optionally wherein the IL-15Rα protein comprises, consists, or consists essentially of an amino acid sequence that is at least 80, 85, 90, 95, 98, or 100% identical to SEQ ID NO: 87 or 88 and retains the T2A substitution. In some embodiments, the IL-15Rα protein comprises or retains one or more amino acid substitutions at position R24, R26, and R35 as defined by SEQ ID NO: 82 (IL-15Rα Sushi+). In some embodiments, the one or more amino acid substitutions are selected from R24E, R26E, and R35E. In some embodiments, the one or more amino acid substitutions are combinations selected from R26E and R35E; and R24E, R26E, and R35. In some embodiments, the IL-15Rα protein comprises or retains an amino acid substitutions at position T2 as defined by SEQ ID NO: 82 (IL-15Rα Sushi+). In some embodiments, the amino acid substitution is T2A. In some embodiments, the IL-15α protein comprises SEQ ID NO: 82 or 83 with the T2A substitution.

In some embodiments, the hinge of the first polypeptide forms at least one or two disulfide bonds with the hinge of the second polypeptide. In some embodiments, the first linker is a non-cleavable or stable linker, and wherein the cleavable linker comprises a protease cleavage site, optionally wherein the cleavable linker is selected from Table S3.

In some embodiments, the protease cleavage site is cleavable by a protease selected from one or more of a metalloprotease, a serine protease, a cysteine protease, and an aspartic acid protease. In some embodiments, protease cleavage site is cleavable by a protease selected from one or more of MMP1, MMP2, MMP3, MMP4, MMP5, MMP6, MMP7, MMP8, MMP9, MMP10, MMP11, MMP12, MMP13, MMP14, TEV protease, matriptase, uPA, FAP, Legumain, PSA, Kallikrein, Cathepsin A, and Cathepsin B. In some embodiments, the first linker and/or the second linker are about 1-50 1-40, 1-30, 1-20, 1-10, 1-5, 1-4, 1-3 amino acids in length, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 amino acids in length.

In some embodiments, the Fab comprises SEQ ID NOs: 3 (VH) and a human IgG1 CH1 domain, and SEQ ID NO:4 (VL) and a CL domain (human kappa); the Fc domain comprises the IgG1 hinge of SEQ ID NO: 42, a modified human IgG1 CH2 domain of SEQ ID NO: 57, and a human IgG1 CH3 domain of SEQ ID NO: 58; the first linker is an 8 amino acid stable linker of SEQ ID NO: (178, wherein x is 2); the IL-15 protein comprises SEQ ID NO: 79, optionally with K86G and S162A mutations; the second linker is a protease cleavable linker of SEQ ID NO: 90 or SEQ ID NO: 201; and the IL-15Rα protein comprises SEQ ID NO: 87, optionally with a T2A mutation.

In some embodiments, cleavage, optionally protease cleavage, of the second linker exposes the binding site(s) of the IL-15 proteins that bind to the IL-15β/γc chain present on the surface of the immune cell in vitro or in vivo. In some embodiments, the immune cell is selected from one or more of a T cell, a B cell, a natural killer cell, a monocyte, and a macrophage.

In some embodiments, the first polypeptide and the second polypeptide comprise, consist, or consist essentially of an amino acid sequence that is at least 80, 85, 90, 95, 98, or 100% identical to a sequence selected from Table S4 (chains 1 and 2), and a VL/CL region polypeptide that is at least 80, 85, 90, 95, 98, or 100% identical to the corresponding sequence from Table S4 (chains 3 and 4), including wherein
the first polypeptide and the second polypeptide comprise, consist, or consist essentially of an amino acid sequence that is at least 80, 85, 90, 95, 98, or 100% identical to SEQ ID NO: 136, and a VL/CL region polypeptide that is at least 80, 85, 90, 95, 98, or 100% identical to SEQ ID NO: 137;
the first polypeptide and the second polypeptide comprise, consist, or consist essentially of an amino acid sequence that is at least 80, 85, 90, 95, 98, or 100% identical to SEQ ID NO: 138, and a VL/CL region polypeptide that is at least 80, 85, 90, 95, 98, or 100% identical to SEQ ID NO: 139;
the first polypeptide and the second polypeptide comprise, consist, or consist essentially of an amino acid sequence that is at least 80, 85, 90, 95, 98, or 100% identical to SEQ ID NO: 140, and a VL/CL region polypeptide that is at least 80, 85, 90, 95, 98, or 100% identical to SEQ ID NO: 141;
the first polypeptide and the second polypeptide comprise, consist, or consist essentially of an amino acid sequence that is at least 80, 85, 90, 95, 98, or 100% identical to SEQ ID NO: 142, and a VL/CL region polypeptide that is at least 80, 85, 90, 95, 98, or 100% identical to SEQ ID NO: 143;
the first polypeptide and the second polypeptide comprise, consist, or consist essentially of an amino acid sequence that is at least 80, 85, 90, 95, 98, or 100% identical to SEQ ID NO: 144, and a VL/CL region polypeptide that is at least 80, 85, 90, 95, 98, or 100% identical to SEQ ID NO: 145;
the first polypeptide and the second polypeptide comprise, consist, or consist essentially of an amino acid sequence that is at least 80, 85, 90, 95, 98, or 100% identical to SEQ ID NO: 146, and a VL/CL region polypeptide that is at least 80, 85, 90, 95, 98, or 100% identical to SEQ ID NO: 147;
the first polypeptide and the second polypeptide comprise, consist, or consist essentially of an amino acid sequence that is at least 80, 85, 90, 95, 98, or 100% identical to SEQ ID NO: 148, and a VL/CL region polypeptide that is at least 80, 85, 90, 95, 98, or 100% identical to SEQ ID NO: 149; or
the first polypeptide and the second polypeptide comprise, consist, or consist essentially of an amino acid sequence that is at least 80, 85, 90, 95, 98, or 100% identical to SEQ ID NO: 199, and a VL/CL region polypeptide that is at least 80, 85, 90, 95, 98, or 100% identical to SEQ ID NO: 200.

Certain activatable proprotein homodimers described herein are substantially in homodimeric form in a physiological solution, or under physiological conditions, optionally in vivo conditions.

Also included are one or more recombinant nucleic acid molecules that encode the activatable proprotein homodimers described herein. In some embodiments, a first recombinant nucleic acid molecule encodes the VH/CH1 regions of the Fab region, the hinge/Fc domain, the first linker, the IL-15 protein, the second linker, and the IL-15Rα protein, and a second nucleic acid molecule encodes the VL/CL regions of the Fab region. Also included are or more vectors comprising the one or more recombinant nucleic acid molecules described herein. Certain embodiments include a host cell comprising the one or more recombinant nucleic acid molecules described herein, or the one or more vectors described herein.

Certain embodiments include methods of producing an activatable proprotein, comprising culturing the host cell described herein under culture conditions suitable for the expression of the activatable proprotein homodimer, and isolating the activatable proprotein from the culture.

Certain embodiments include methods of producing an activatable proprotein, comprising culturing a host cell described herein under culture conditions suitable for the expression of the activatable proprotein homodimer, and isolating the activatable proprotein from the culture.

Also included are pharmaceutical compositions, comprising an activatable proprotein homodimer described herein, and a pharmaceutically acceptable carrier.

Certain embodiments relate to methods of treating disease in a subject, and/or methods of enhancing an immune response in a subject, comprising administering to the subject a therapeutically effective amount of the pharmaceutical composition described herein.

In some embodiments, the disease is a cancer, for instance, a cancer that expresses or over-expresses PD-L1 or B7H3. In some embodiments, the cancer is a primary cancer or a metastatic cancer, and is selected from one or more of melanoma (optionally metastatic melanoma), kidney cancer (optionally renal cell carcinoma), pancreatic cancer, bone cancer, prostate cancer, small cell lung cancer, non-small cell lung cancer (NSCLC), mesothelioma, leukemia (optionally lymphocytic leukemia, chronic myelogenous leukemia, acute myeloid leukemia, or relapsed acute myeloid leukemia), multiple myeloma, lymphoma, hepatoma (hepatocellular carcinoma), sarcoma, B-cell malignancy, breast cancer, ovarian cancer, colorectal cancer, glioma, glioblastoma multiforme, meningioma, pituitary adenoma, vestibular schwannoma, primary CNS lymphoma, primitive neuroectodermal tumor (medulloblastoma), bladder cancer, uterine cancer, esophageal cancer, brain cancer, head and neck cancers, cervical cancer, testicular cancer, thyroid cancer, and stomach cancer.

In certain embodiments, following administration, the activatable proprotein homodimer is activated through protease cleavage in a cancer cell or cancer tissue, or a tumor microenvironment (TME), which exposes the binding site(s) of the IL-15 proteins that bind to the IL-15β/γc chain present on the surface of the immune cell in vitro or in vivo, and thereby generates an activated protein. In some embodiments, the activated protein binds via the IL-15 protein to the 15β/γc chain present on the surface of an immune cell in vitro or in vivo. In some embodiments, the immune cell is selected from one or more of a T cell, a B cell, a natural killer cell, a monocyte, and a macrophage.

In some embodiments, administration and activation of the activatable proprotein increases an anti-cancer immune response in the subject by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control. In some embodiments, administration and activation of the activatable proprotein increases cancer cell-killing in the subject by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control.

In some embodiments, the pharmaceutical composition is administered to the subject by parenteral administration. In some embodiments, the parenteral administration is intravenous administration.

Certain embodiments include the use of a pharmaceutical composition described herein in the preparation of a medicament for treating a disease in a subject, optionally cancer (e.g., PD-L1 expressing or over-expression cancer), and/or for enhancing an immune response in a subject. Certain embodiments include a pharmaceutical composition described herein for use in treating a disease in a subject, optionally cancer (e.g., PD-L1 or B7H3 expressing or over-expression cancer), and/or for enhancing an immune response in a subject.

### BRIEF DESCRIPTION OF THE DRAWINGS

FIGS. 1A-1B show exemplary structures of a single polypeptide (IgG-proIL-15 motif), which forms a homodimer with a second polypeptide having the same structure. The IgG-proIL-15 motif is comprised of a high affinity neutralizing antibody (for example, with no FcγR binding and intact FcRn binding) and an IL-15 procytokine module.

FIG. 2 shows an exemplary structure of an activatable proprotein homodimer in its inactive (procytokine) state. The dashed lines indicate a cleavable linker. The intact IgG-proIL-15 construct shows little or no IL-15 activity because the IL-15βγ binding site is ‘masked’ in this format.

FIG. 3 illustrates the IL-15 “activation” of the homodimer by protease cleavage of the cleavable linker in the TME. The protease cleavable linkers between IL-15 and IL-15Rα are stable in the peripheral blood, but can be cleaved by tumor proteases in the TME, thereby releasing the active IL-15 at the tumor site. Such can reduce the toxicity in peripheral blood and increase the anti-tumor activity.

FIGS. 4A-4B show the ELISA binding activity of (A) PD-1-proIL-15 to human PD-1 in comparison to PD-1 IgG, and (B) PD-L1-proIL-15 to PD-L1 in comparison to PD-L1 IgG.

FIGS. 5A-5B show proliferation of the human acute megakaryoblastic leukemia cell line M-07e induced by (A) protease activated PD-1-proIL-15 in comparison to human recombinant IL-15, and (B) protease activated PD-L1-proIL-15 in comparison to human recombinant IL-15.

FIGS. 6A-6D show the results of a STAT5 phosphorylation assay of intact and protease activated PD-1-proIL-15 on resting PBMCs of donor 1 (CD4 T-cells (A), CD8 T-cells (B), regulatory T-cell (C), and NK cells (D); Key on 6B).

FIGS. 7A-7D show the results of a STAT5 phosphorylation assay of intact and protease activated PD-1-IL-15 on activated PBMCs of donor 1 (CD4 T-cells (A), CD8 T-cells (B), regulatory T-cell (C), and NK cells (D); Key on 7B).

FIGS. 8A-8D show the results of a STAT5 phosphorylation assay of intact and protease activated PD-L1-IL-15 on resting PBMCs of donor 2 (CD4 T-cells (A), CD8 T-cells (B), regulatory T-cell (C), and NK cells (D); Key on 8B).

FIG. 9 shows the dose dependent IFN-γ secretion by PBMCs from donor 1 upon 3 days of in vitro stimulation with increasing concentrations of both intact and protease activated PD-1-proIL-15 and PD-L1-proIL-15.

FIG. 10A shows the averaged tumor volume measured over time in A375-PBMC xenograft model, which evidences the in vivo anti-tumor activities of B7H3-proIL-15 with different protease cleavable linkers as single agents in inhibiting tumor growth. FIG. 10B shows the averaged tumor volume at Day 22 for the same treatments in 10A. Mice (n=6) were i.v. injected at Day 0, 3, 7, 10, and 14; the results are expressed as mean±S.E.M.

FIG. 11A shows the averaged tumor volume measured over time in A375-PBMC xenograft model, which evidences the in vivo dose-dependent anti-tumor activities of PD-L1-proIL-15 as a single agent in inhibiting the tumor growth. FIG. 11B shows the averaged tumor volume measured over time in A375-PBMC xenograft model, which evidences the in vivo dose-dependent anti-tumor activities of PD-1-proIL-15 as a single agent in inhibiting tumor growth. Mice (n=6) were i.v. injected at Day 0, 7, and 14; the results are expressed as mean±S.E.M.

FIG. 12 shows the averaged tumor volume measured over time in the B16F10 syngeneic model, which evidences the in vivo anti-tumor activities of mPD-1-proIL-15 (P55654367) as a single agent in inhibiting tumor growth. Mice (n=6) were i.v. injected at Day 0 and 3; the results are expressed as mean±S.E.M.

FIG. 13A shows the concentration of PD-1-proIL-15 over time in peripheral blood of cynomolgus monkeys. FIGS. 13B-13D show the expression of Ki67 in NK (B), CD4 (C), and CD8 (D) cells over time in peripheral blood of cynomolgus monkeys. FIGS. 13E-13G show the fold change of cell number of NK (E), CD4+(F), and CD8+(G) cells over time in peripheral blood of cynomolgus monkeys. Monkeys (n=2) were i.v. injected with P53052037 10 mg/kg, Q2W. Circles=male; squares=female.

FIGS. 14A-14D show STAT5 phosphorylation in CD4 T-cells (14A), CD8 T-cells (14B), regulatory T-cells (14C) and NK cells (14D) upon treatment of resting PBMCs with rhIL-15 as well as intact and activated PD-1-proIL-15. The proprotein form of P79772037 was unable to induce STAT5 phosphorylation in all tested cell subsets. The activated form of P79772037 (matrix metalloproteinase-2 cleaved) was equally effective in activating STAT5 phosphorylation in CD8 and CD4 T cells and less potent in NK cells.

FIGS. 15A-15G show the purification and characterization of PD-1-proIL-15 (P53052037, P79772037), PD-L1-proIL-15 (P53021942), and B7H3-proIL-15 (P40503699, P40743699). FIG. 15A shows the reduced SDS-PAGE analysis results, and FIG. 15B shows the non-reduced SDS-PAGE analysis results. FIGS. 15C-15G shows the size exclusion chromatography (SEC-HPLC) analysis results indicating high purity and homogeneity of products (without significant amount of aggregation or degradation product).

### DETAILED DESCRIPTION

Embodiments of the present disclosure relate to activatable proprotein homodimers, comprising two separate but identical polypeptides, each polypeptide comprising in an N- to C-terminal orientation, a fragment antigen-binding (Fab) region that specifically binds to human PD-1 or human PD-L1 or human B7H3, a hinge/Fc domain, a stable linker, an IL-15 protein, a protease cleavable linker, and an IL-15Rα protein. In some instances, the homodimer is formed by the following binding interactions: binding of the hinge/Fc domain of the first polypeptide to the hinge/Fc domain of the second polypeptide, and binding of each of the IL-15 proteins of one polypeptide to each of the IL-15Rα proteins of the other polypeptide. These binding interactions form a biologically-inactive (proprotein) homodimer by masking the binding sites of the IL-15 proteins that would otherwise bind to an IL-15β/γc chain present on the surface of an immune cell. The proprotein homodimer remains inactive or substantially inactive in plasma.

In certain instances, the homodimer is targeted to the tumor microenvironment (TME) by the anti-PD-1 or anti-PD-L1 Fab, and activated within the TME by exposure to tumor-site proteases, which cleave the protease cleavable linker, thereby reactivating the IL-15 protein(s) (see, for example, WO 2020/123980). Such allows synergy between the anti-tumor activity of the anti-PD-1/anti-PD-L1 Fab, and the immune-stimulating activity of the IL-15 proteins. In certain instances, the homodimer is targeted to the tumor microenvironment (TME) by the anti-B7H3 Fab, and activated within the TME by exposure to tumor-site proteases, which cleave the protease cleavable linker, thereby reactivating the IL-15 protein(s) (see, for example, WO 2020/123980). Such allows synergy between the anti-tumor activity of the anti-B7H3 Fab, and the immune-stimulating activity of the IL-15 proteins.

Unless defined otherwise, all technical and scientific terms used herein have the same meaning as commonly understood by those of ordinary skill in the art to which the disclosure belongs. Although any methods, materials, compositions, reagents, cells, similar or equivalent similar or equivalent to those described herein can be used in the practice or testing of the subject matter of the present disclosure, preferred methods and materials are described. All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. These and related techniques and procedures may be generally performed according to conventional methods well known in the art and as described in various general and more specific references that are cited and discussed throughout the present specification. Unless specific definitions are provided, the nomenclature utilized in connection with, and the laboratory procedures and techniques of, molecular biology, analytical chemistry, synthetic organic chemistry, and medicinal and pharmaceutical chemistry described herein are those well-known and commonly used in the art. Standard techniques may be used for recombinant technology, molecular biological, microbiological, chemical syntheses, chemical analyses, pharmaceutical preparation, formulation, and delivery, and treatment of patients.

For the purposes of the present disclosure, the following terms are defined below.

The articles “a” and “an” are used herein to refer to one or to more than one (i.e., to at least one) of the grammatical object of the article. By way of example, “an element” includes “one element”, “one or more elements” and/or “at least one element”.

By “about” is meant a quantity, level, value, number, frequency, percentage, dimension, size, amount, weight or length that varies by as much as 20, 15, 10, 9, 8, 7, 6, 5, 4, 3, 2 or 1% to a reference quantity, level, value, number, frequency, percentage, dimension, size, amount, weight or length.

The terms “activatable proprotein,” “proprotein”, “activatable procytokine”, “procytokine”, “activatable prodrug”, and “prodrug” or are used interchangeably herein and refer to an activatable proprotein comprising at least a masking moiety and an active domain, or derivatives/variants therefrom, as described herein. In one embodiment, the proprotein may also comprise one or more protein domains.

The term “antigen” refers to a molecule or a portion of a molecule capable of being bound by a selective binding agent, such as an antibody, and additionally capable of being used in an animal to produce antibodies capable of binding to an epitope of that antigen. An antigen may have one or more epitopes. As used herein, the term “antigen” includes substances that are capable, under appropriate conditions, of inducing an immune response to the substance and of reacting with the products of the immune response. More broadly, the term “antigen” includes any substance to which an antibody binds, or for which antibodies are desired, regardless of whether the substance is immunogenic. For such antigens, antibodies can be identified by recombinant methods, independently of any immune response.

An “antagonist” refers to biological structure or chemical agent that interferes with or otherwise reduces the physiological action of another agent or molecule. In some instances, the antagonist specifically binds to the other agent or molecule. Included are full and partial antagonists.

An “agonist” refers to biological structure or chemical agent that increases or enhances the physiological action of another agent or molecule. In some instances, the agonist specifically binds to the other agent or molecule. Included are full and partial agonists.

As used herein, the term “amino acid” is intended to mean both naturally occurring and non-naturally occurring amino acids as well as amino acid analogs and mimetics. Naturally-occurring amino acids include the 20 (L)-amino acids utilized during protein biosynthesis as well as others such as 4-hydroxyproline, hydroxylysine, desmosine, isodemosine, homocysteine, citrulline and ornithine, for example. Non-naturally occurring amino acids include, for example, (D)-amino acids, norleucine, norvaline, p-fluorophenylalanine, ethionine and the like, which are known to a person skilled in the art. Amino acid analogs include modified forms of naturally and non-naturally occurring amino acids.

Such modifications can include, for example, substitution or replacement of chemical groups and moieties on the amino acid or by derivatization of the amino acid. Amino acid mimetics include, for example, organic structures which exhibit functionally similar properties such as charge and charge spacing characteristic of the reference amino acid. For example, an organic structure which mimics arginine (Arg or R) would have a positive charge moiety located in similar molecular space and having the same degree of mobility as the e-amino group of the side chain of the naturally occurring Arg amino acid. Mimetics also include constrained structures so as to maintain optimal spacing and charge interactions of the amino acid or of the amino acid functional groups. Those skilled in the art know or can determine what structures constitute functionally equivalent amino acid analogs and amino acid mimetics.

As used herein, a subject “at risk” of developing a disease, or adverse reaction may or may not have detectable disease, or symptoms of disease, and may or may not have displayed detectable disease or symptoms of disease prior to the treatment methods described herein. “At risk” denotes that a subject has one or more risk factors, which are measurable parameters that correlate with development of a disease, as described herein and known in the art. A subject having one or more of these risk factors has a higher probability of developing disease, or an adverse reaction than a subject without one or more of these risk factor(s).

“Biocompatible” refers to materials or compounds which are generally not injurious to biological functions of a cell or subject and which will not result in any degree of unacceptable toxicity, including allergenic and disease states.

The term “binding” refers to a direct association between two molecules, due to, for example, covalent, electrostatic, hydrophobic, and ionic and/or hydrogen-bond interactions, including interactions such as salt bridges and water bridges.

By “coding sequence” is meant any nucleic acid sequence that contributes to the code for the polypeptide product of a gene. By contrast, the term “non-coding sequence” refers to any nucleic acid sequence that does not directly contribute to the code for the polypeptide product of a gene.

Throughout this disclosure, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The term “endotoxin free” or “substantially endotoxin free” relates generally to compositions, solvents, and/or vessels that contain at most trace amounts (e.g., amounts having no clinically adverse physiological effects to a subject) of endotoxin, and preferably undetectable amounts of endotoxin. Endotoxins are toxins associated with certain micro-organisms, such as bacteria, typically gram-negative bacteria, although endotoxins may be found in gram-positive bacteria, such as Listeria monocytogenes. The most prevalent endotoxins are lipopolysaccharides (LPS) or lipo-oligo-saccharides (LOS) found in the outer membrane of various Gram-negative bacteria, and which represent a central pathogenic feature in the ability of these bacteria to cause disease. Small amounts of endotoxin in humans may produce fever, a lowering of the blood pressure, and activation of inflammation and coagulation, among other adverse physiological effects.

Therefore, in pharmaceutical production, it is often desirable to remove most or all traces of endotoxin from drug products and/or drug containers, because even small amounts may cause adverse effects in humans. A depyrogenation oven may be used for this purpose, as temperatures in excess of 300° C. are typically required to break down most endotoxins. For instance, based on primary packaging material such as syringes or vials, the combination of a glass temperature of 250° C. and a holding time of 30 minutes is often sufficient to achieve a 3 log reduction in endotoxin levels. Other methods of removing endotoxins are contemplated, including, for example, chromatography and filtration methods, as described herein and known in the art.

Endotoxins can be detected using routine techniques known in the art. For example, the Limulus Amoebocyte Lysate assay, which utilizes blood from the horseshoe crab, is a very sensitive assay for detecting presence of endotoxin. In this test, very low levels of LPS can cause detectable coagulation of the limulus lysate due a powerful enzymatic cascade that amplifies this reaction. Endotoxins can also be quantitated by enzyme-linked immunosorbent assay (ELISA). To be substantially endotoxin free, endotoxin levels may be less than about 0.001, 0.005, 0.01, 0.02, 0.03, 0.04, 0.05, 0.06, 0.08, 0.09, 0.1, 0.5, 1.0, 1.5, 2, 2.5, 3, 4, 5, 6, 7, 8, 9, or 10 EU/mg of active compound. Typically, 1 ng lipopolysaccharide (LPS) corresponds to about 1-10 EU.

The term “half maximal effective concentration” or “EC₅₀” refers to the concentration of an agent (e.g., activatable proprotein) as described herein at which it induces a response halfway between the baseline and maximum after some specified exposure time; the EC₅₀ of a graded dose response curve therefore represents the concentration of a compound at which 50% of its maximal effect is observed. EC₅₀ also represents the plasma concentration required for obtaining 50% of a maximum effect in vivo. Similarly, the “EC₉₀” refers to the concentration of an agent or composition at which 90% of its maximal effect is observed. The “EC₉₀” can be calculated from the “EC₅₀” and the Hill slope, or it can be determined from the data directly, using routine knowledge in the art. In some embodiments, the EC₅₀ of an agent (e.g., activatable proprotein) is less than about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90, 100, 200 or 500 nM. In some embodiments, an agent will have an EC₅₀ value of about 1 nM or less.

“Immune response” means any immunological response originating from immune system, including responses from the cellular and humeral, innate and adaptive immune systems. Exemplary cellular immune cells include for example, lymphocytes, macrophages, T cells, B cells, NK cells, neutrophils, eosinophils, dendritic cells, mast cells, monocytes, and all subsets thereof. Cellular responses include for example, effector function, cytokine release, phagocytosis, efferocytosis, translocation, trafficking, proliferation, differentiation, activation, repression, cell-cell interactions, apoptosis, etc. Humeral responses include for example IgG, IgM, IgA, IgE, responses and their corresponding effector functions.

The “half-life” of an agent such as an activatable proprotein can refer to the time it takes for the agent to lose half of its pharmacologic, physiologic, or other activity, relative to such activity at the time of administration into the serum or tissue of an organism, or relative to any other defined time-point. “Half-life” can also refer to the time it takes for the amount or concentration of an agent to be reduced by half of a starting amount administered into the serum or tissue of an organism, relative to such amount or concentration at the time of administration into the serum or tissue of an organism, or relative to any other defined time-point. The half-life can be measured in serum and/or any one or more selected tissues.

The terms “modulating” and “altering” include “increasing” or “enhancing” as well as “decreasing” or “reducing,” typically in a statistically significant or a physiologically significant amount or degree relative to a control. An “increased” or “enhanced” amount is typically a “statistically significant” amount, and may include an amount that is about or at least about 1.1, 1.2, 1.5, 2, 3, 4, 5, 6, 7, 8, 9, 10, 15, 20, 30, 40, 50, 60, 70, 80, 90, 100, 200, 300, 400, 500, 600, 700, 800, 900, 1000, 2000, 3000, 4000, 5000-fold or more relative to a control. An “increased” or “enhanced” amount may also include an amount that is about or at least about 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, 2000%, 3000%, 4000%, 5000% or more of the amount relative to a control. A “decreased” or “reduced” amount is typically a “statistically significant” amount, and may include an amount that is about or at least about 1.1, 1.2, 1.5, 2, 3, 4, 5, 6, 7, 8, 9, 10, 15, 20, 30, 40, 50, 60, 70, 80, 90, 100, 200, 300, 400, 500, 600, 700, 800, 900, 1000, 2000, 3000, 4000, or 5000-fold less of the amount relative to a control. A “decreased” or “reduced” amount may also include a 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100%, 200%, 300%, 400%, 500%, 600%, 700%, 800%, 900%, 1000%, 2000%, 3000%, 4000%, or 5000% less of the amount relative to a control. Examples of comparisons and “statistically significant” amounts are described herein.

The terms “polypeptide,” “protein” and “peptide” are used interchangeably and mean a polymer of amino acids not limited to any particular length. The term “enzyme” includes polypeptide or protein catalysts. The terms include modifications such as myristoylation, sulfation, glycosylation, phosphorylation and addition or deletion of signal sequences. The terms “polypeptide” or “protein” means one or more chains of amino acids, wherein each chain comprises amino acids covalently linked by peptide bonds, and wherein said polypeptide or protein can comprise a plurality of chains non-covalently and/or covalently linked together by peptide bonds, having the sequence of native proteins, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence. In certain embodiments, the polypeptide is a “recombinant” polypeptide, produced by recombinant cell that comprises one or more recombinant DNA molecules, which are typically made of heterologous polynucleotide sequences or combinations of polynucleotide sequences that would not otherwise be found in the cell.

The term “polynucleotide” and “nucleic acid” includes mRNA, RNA, cRNA, cDNA, and DNA. The term typically refers to polymeric form of nucleotides of at least 10 bases in length, either ribonucleotides or deoxynucleotides or a modified form of either type of nucleotide. The term includes single and double stranded forms of DNA. The terms “isolated DNA” and “isolated polynucleotide” and “isolated nucleic acid” refer to a molecule that has been isolated free of total genomic DNA of a particular species. Therefore, an isolated DNA segment encoding a polypeptide refers to a DNA segment that contains one or more coding sequences yet is substantially isolated away from, or purified free from, total genomic DNA of the species from which the DNA segment is obtained. Also included are non-coding polynucleotides (e.g., primers, probes, oligonucleotides), which do not encode a polypeptide. Also included are recombinant vectors, including, for example, expression vectors, viral vectors, plasmids, cosmids, phagemids, phage, viruses, and the like.

Additional coding or non-coding sequences may, but need not, be present within a polynucleotide described herein, and a polynucleotide may, but need not, be linked to other molecules and/or support materials. Hence, a polynucleotide or expressible polynucleotides, regardless of the length of the coding sequence itself, may be combined with other sequences, for example, expression control sequences.

The term “isolated” polypeptide or protein referred to herein means that a subject protein (1) is free of at least some other proteins with which it would typically be found in nature, (2) is essentially free of other proteins from the same source, e.g., from the same species, (3) is expressed by a cell from a different species, (4) has been separated from at least about 50 percent of polynucleotides, lipids, carbohydrates, or other materials with which it is associated in nature, (5) is not associated (by covalent or non-covalent interaction) with portions of a protein with which the “isolated protein” is associated in nature, (6) is operably associated (by covalent or non-covalent interaction) with a polypeptide with which it is not associated in nature, or (7) does not occur in nature. Such an isolated protein can be encoded by genomic DNA, cDNA, mRNA or other RNA, of may be of synthetic origin, or any combination thereof. In certain embodiments, the isolated protein is substantially free from proteins or polypeptides or other contaminants that are found in its natural environment that would interfere with its use (therapeutic, diagnostic, prophylactic, research or otherwise).

In certain embodiments, the “purity” of any given agent (e.g., activatable proprotein) in a composition may be defined. For instance, certain compositions may comprise an agent such as a polypeptide agent that is at least 80%, 85%, 90%, 91%, 92%, 93%, 94%, 95%, 96%, 97%, 98%, 99%, or 100% pure on a protein basis or a weight-weight basis, including all decimals and ranges in between, as measured, for example and by no means limiting, by high performance liquid chromatography (HPLC), a well-known form of column chromatography used frequently in biochemistry and analytical chemistry to separate, identify, and quantify compounds.

The term “reference sequence” refers generally to a nucleic acid coding sequence, or amino acid sequence, to which another sequence is being compared. All polypeptide and polynucleotide sequences described herein are included as references sequences, including those described by name and those described in the Tables and the Sequence Listing.

Certain embodiments include biologically active “variants” and “fragments” of the proteins/polypeptides described herein, and the polynucleotides that encode the same. “Variants” contain one or more substitutions, additions, deletions, and/or insertions relative to a reference polypeptide or polynucleotide (see, e.g., the Tables and the Sequence Listing). A variant polypeptide or polynucleotide comprises an amino acid or nucleotide sequence with at least about 50%, 55%, 60%, 65%, 70%, 75%, 80%, 85%, 90%, 91%, 92%, 93%, 94%, 95%, 96%, 97%, 98%, 99% or more sequence identity or similarity or homology to a reference sequence, as described herein, and substantially retains the activity of that reference sequence. Also included are sequences that consist of or differ from a reference sequences by the addition, deletion, insertion, or substitution of 1, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130, 140, 150 or more amino acids or nucleotides and which substantially retain at least one activity of that reference sequence. In certain embodiments, the additions or deletions include C-terminal and/or N-terminal additions and/or deletions.

The terms “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., Nucl. Acids Res. 25:3389, 1997.

The term “solubility” refers to the property of an agent (e.g., activatable proprotein) provided herein to dissolve in a liquid solvent and form a homogeneous solution. Solubility is typically expressed as a concentration, either by mass of solute per unit volume of solvent (g of solute per kg of solvent, g per dL (100 mL), mg/ml, etc.), molarity, molality, mole fraction or other similar descriptions of concentration. The maximum equilibrium amount of solute that can dissolve per amount of solvent is the solubility of that solute in that solvent under the specified conditions, including temperature, pressure, pH, and the nature of the solvent. In certain embodiments, solubility is measured at physiological pH, or other pH, for example, at pH 5.0, pH 6.0, pH 7.0, pH 7.4, pH 7.6, pH 7.8, or pH 8.0 (e.g., about pH 5-8). In certain embodiments, solubility is measured in water or a physiological buffer such as PBS or NaCl (with or without NaPO₄). In specific embodiments, solubility is measured at relatively lower pH (e.g., pH 6.0) and relatively higher salt (e.g., 500 mM NaCl and 10 mM NaPO₄). In certain embodiments, solubility is measured in a biological fluid (solvent) such as blood or serum. In certain embodiments, the temperature can be about room temperature (e.g., about 20, 21, 22, 23, 24, 25° C.) or about body temperature (37° C.). In certain embodiments, an agent has a solubility of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90 or 100 mg/ml at room temperature or at 37° C.

A “subject” or a “subject in need thereof” or a “patient” or a “patient in need thereof” includes a mammalian subject such as a human subject.

“Substantially” or “essentially” means nearly totally or completely, for instance, 95%, 96%, 97%, 98%, 99% or greater of some given quantity.

By “statistically significant,” it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

“Therapeutic response” refers to improvement of symptoms (whether or not sustained) based on administration of one or more therapeutic agents.

As used herein, the terms “therapeutically effective amount”, “therapeutic dose,” “prophylactically effective amount,” or “diagnostically effective amount” is the amount of an agent (e.g., activatable proprotein, activated protein) needed to elicit the desired biological response following administration.

As used herein, “treatment” of a subject (e.g., a mammal, such as a human) or a cell is any type of intervention used in an attempt to alter the natural course of the individual or cell. Treatment includes, but is not limited to, administration of a pharmaceutical composition, and may be performed either prophylactically or subsequent to the initiation of a pathologic event or contact with an etiologic agent. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

The term “wild-type” refers to a gene or gene product (e.g., a polypeptide) that is most frequently observed in a population and is thus arbitrarily designed the “normal” or “wild-type” form of the gene.

Each embodiment in this specification is to be applied to every other embodiment unless expressly stated otherwise.

### Activatable Proprotein Homodimers

Certain embodiments relate to activatable proprotein homodimers, comprising a first polypeptide and a second polypeptide, wherein the first polypeptide and the second polypeptide comprise, in an N- to C-terminal orientation, a fragment antigen-binding (Fab) region that specifically binds to human PD-1 or human PD-L1 or human B7H3, a hinge/Fc domain, a first linker, an IL-15 protein, a second linker, and an IL-15Rα protein, wherein the hinge/Fc domain of the first polypeptide binds to the hinge/Fc domain of the second polypeptide, wherein the IL-15 protein of the first polypeptide binds to the IL-15Rα protein of the second polypeptide, and wherein the IL-15Rα of the first polypeptide binds to the IL-15 protein of the second polypeptide, wherein said binding masks a binding site of the IL-15 protein(s) that otherwise binds to an IL-15β/γc chain present on the surface of an immune cell in vitro or in vivo, and wherein the second linker is a cleavable linker.

As noted above, the IL-15 protein(s) and the IL-15Rα protein(s) interact or bind together, for example, via non-covalent interactions or certain covalent bonds (e.g., disulfide bonds). In some instances, the binding of the IL-15 protein(s) to the IL-15Rα protein(s) sterically blocks or hinders binding of the IL-15 protein(s) to their cognate IL-15β/γc receptor chains expressed on immune cells. Exemplary IL-15 proteins and IL-15Rα proteins are described elsewhere herein.

In some instances, the hinge/Fc domains of the first and second polypeptides dimerize together via at least one non-covalent interaction, at least one covalent bond (for example, at least one disulfide bond), or any combination of non-covalent interactions and covalent bonds, to further stabilize the activatable proprotein and/or to further mask the binding of the IL-15 proteins to their cognate receptors, for example, IL-15β/γc receptor chains. Typically, however, the hinge/Fc domains of the first and second polypeptide do not bind together or dimerize via a peptide or amide bond. In some embodiments, the hinge/Fc domains bind together as a homodimer, that is, a homodimer composed of two identical or nearly identical hinge/Fc domains. Thus, the hinge/Fc domains of the first and second polypeptides can be the same (or substantially the same) or different (e.g., knob-in-hole). Exemplary hinge/Fc domains are described herein.

As noted above, the second linker comprises a cleavable linker, for example, a linker cleavable by a protease. In some instances, the first linker is a stable (e.g., physiologically stable) linker. In some instances, the first linker is also a cleavable linker, for example, a linker cleavable by a protease. In some instances, the protease is expressed in target tissues or cells, for example, cancer tissues or cancer cells. Cleavage of the linker in that context releases a masking moiety, removes the steric hindrance of the IL-15 protein, and allows selective activation of the IL-15 protein in diseased tissues or cells (e.g., TME), relative to normal or healthy tissues or cells. Such selective and localized activation not only reduces needless consumption of administered IL-15, thereby increasing its half-life, but also enhances tissue penetration and reduces undesirable systemic effects of IL-15, among other advantages. Exemplary linkers are described herein.

In some embodiments, the homodimeric binding between the first and second polypeptides allosterically inhibits the binding of the IL-15 proteins to their target, for example, cognate IL-15β/γc receptor chains on the surface of an immune cell. In these and related embodiments, the IL-15 portion of the activatable proprotein shows no binding or substantially no binding to its target, or no more than 0.001%, 0.01%, 0.1%, 1%, 2%, 3%, 4%, 5%, 6%, 7%, 8%, 9%, 10%, 15%, 20%, 25%, 30%, 35%, 40%, or 50% binding to its target, as compared to the binding of the active domain or the IL-15 protein alone, optionally for at least 2, 4, 6, 8, 12, 28, 24, 30, 36, 48, 60, 72, 84, 96 hours, or 5, 10, 15, 30, 45, 60, 90, 120, 150, 180 days, or 1, 2, 3, 4, 5, 6, 7, 8, 9, 10, 11, 12 months or greater, optionally as measured in vivo or in a Target Displacement in vitro assay available in the art.

In certain instances, the anti-PD-1 or anti-PD-L1 or human B7H3 Fab not only improves targeting of the activatable IL-15 proprotein (or procytokine) module to the TME, but also provides increased anti-cancer/immune-stimulating activity in addition to that of the IL-15 protein, once the latter is activated by protease cleavage of the second linker.

In specific embodiments, the first and second polypeptides of the activatable proprotein homodimer comprise an anti-PD-1 Fab (SEQ ID NOs: 3 and 4) or an anti-PD-L1 Fab (SEQ ID NOs: 25 and 26), together with a human IgG1 CH1 domain and a CL domain (kappa), an IgG1 hinge (SEQ ID NO: 42), a modified IgG1 Fc domain with LALA and P329A mutations (see CH2 domain of SEQ ID NO: 57), an IgG1 CH3 domain (see CH3 domain of SEQ ID NO: 58), a stable linker (for example, of eight amino acids such as a GGGSGGGS; SEQ ID NO: 178), an IL-15 protein (SEQ ID NO: 69 or 79 optionally with K86G and S162A mutations), a protease cleavable linker (for example, GGGGSPLGLSGRSDNQGGGGSGGGGS, SEQ ID NO: 90; or GGGSPLGLAGSGRSDNQGGSGGSGGS, SEQ ID NO: 201), and an IL-15Rα protein (SEQ ID NO: 87 or 88 optionally with T2A mutation), including active fragments and variants of the foregoing sequences, as described herein. The individual components of exemplary activatable proproteins are described in greater detail herein.

Anti-PD-1 and Anti-PD-L1 and Anti-B7H3 Fab Regions. The activatable proproteins described herein comprise at least one fragment antigen-binding (Fab) region that specifically binds to human PD-1 or human PD-L1 or human B7H3. A “Fab” region is composed of one constant and one variable domain of each of the heavy and the light chains of an immunoglobulin molecule, for instance, a VL/CL region bound to a VH/CH1 region that is fused at its C-terminus to the Fc domain optionally via a linker or hinge (hinge/Fc domain). The VL:VH and CL:CH1 regions of the Fab are typically bound together as a covalent heterodimer. In certain embodiments, the CL domain is a kappa chain. In some embodiments, the CL domain is a lambda chain. In some embodiments, the CH1 domain is an IgA, IgD, IgE, IgG, IgM domain, for example, an IgA1, IgA2, IgG1, IgG2, IgG2, IgG3, or IgG4 CH1 domain. In specific embodiments, the CL domain is a kappa chain and the CH1 domain is an IgG1 domain.

In certain embodiments, an antibody or Fab region as described herein includes a heavy chain and a light chain CDR set, respectively interposed between a heavy chain and a light chain framework region (FR) set which provide support to the CDRs and define the spatial relationship of the CDRs relative to each other. As used herein, the term “CDR set” refers to the three hypervariable regions of a heavy or light chain V region. Proceeding from the N-terminus of a heavy or light chain, these regions are denoted as “CDR1,” “CDR2,” and “CDR3” respectively. An antigen-binding site, therefore, includes six CDRs, comprising the CDR set from each of a heavy and a light chain V region (VHCDR1, VHCDR2, VHCDR3, VLCDR1, VLCDR2, VLCDR3). A polypeptide comprising a single CDR, (e.g., a CDR1, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRs. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures-regardless of the precise CDR amino acid sequence. Further, certain FR residues are known to participate in non-covalent interdomain contacts which stabilize the interaction of the antibody heavy and light chains.

In certain embodiments, the Fab regions are humanized. These embodiments refer to a chimeric molecule, generally prepared using recombinant techniques, having an antigen-binding site derived from an immunoglobulin from a non-human species and the remaining immunoglobulin structure of the molecule based upon the structure and/or sequence of a human immunoglobulin. The antigen-binding site may comprise either complete variable domains fused onto constant domains or only the CDRs grafted onto appropriate framework regions in the variable domains. Epitope binding sites may be wild-type or modified by one or more amino acid substitutions. This eliminates the constant region as an immunogen in human individuals, but the possibility of an immune response to the foreign variable region remains (LoBuglio et al., PNAS USA 86:4220-4224, 1989; Queen et al., PNAS USA. 86:10029-10033, 1988; Riechmann et al., Nature. 332:323-327, 1988). Illustrative methods for humanization of antibodies include the methods described in U.S. Pat. No. 7,462,697.

Another approach focuses not only on providing human-derived constant regions, but modifying the variable regions as well so as to reshape them as closely as possible to human form. It is known that the variable regions of both heavy and light chains contain three complementarity-determining regions (CDRs) which vary in response to the epitopes in question and determine binding capability, flanked by four framework regions (FRs) which are relatively conserved in a given species and which putatively provide a scaffolding for the CDRs. When nonhuman antibodies are prepared with respect to a particular epitope, the variable regions can be “reshaped” or “humanized” by grafting CDRs derived from nonhuman antibody on the FRs present in the human antibody to be modified. Application of this approach to various antibodies has been reported by Sato et al., Cancer Res. 53:851-856, 1993; Riechmann et al., Nature 332:323-327, 1988; Verhoeyen et al., Science 239:1534-1536, 1988; Kettleborough et al., Protein Engineering. 4:773-3783, 1991; Maeda et al., Human Antibodies Hybridoma 2:124-134, 1991; Gorman et al., PNAS USA. 88:4181-4185, 1991; Tempest et al., Bio/Technology 9:266-271, 1991; Co et al., PNAS USA. 88:2869-2873, 1991; Carter et al., PNAS USA. 89:4285-4289, 1992; and Co et al., J Immunol. 148:1149-1154, 1992. In some embodiments, humanized antibodies or Fab regions preserve all CDR sequences (for example, a humanized mouse antibody or Fab which contains all six CDRs from the mouse antibodies). In certain embodiments, humanized antibodies or Fab regions have one or more CDRs (one, two, three, four, five, six) which are altered with respect to the original antibody, which are also termed one or more CDRs “derived from” one or more CDRs from the original antibody.

The binding properties of antibodies and Fab regions can be quantified using methods well known in the art (see Davies et al., Annual Rev. Biochem. 59:439-473, 1990). In some embodiments, an antibody or Fab region specifically binds to a target molecule, for example, a PD-1 or PD-L1 protein or an epitope or complex thereof, with an equilibrium dissociation constant that is about or ranges from about ≤10⁻⁷ M to about 10⁻⁸ M. In some embodiments, the equilibrium dissociation constant is about or ranges from about ≤10⁻⁹ M to about ≤10⁻¹⁰ M. In certain illustrative embodiments, an antibody or antigen-binding fragment thereof has an affinity (Kd or EC₅₀) for a PD-1 or PD-L1 protein (to which it specifically binds) of about, at least about, or less than about, 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, or 50 nM.

A molecule such as an antibody or Fab region is said to exhibit “specific binding” or “preferential binding” if it reacts or associates more frequently, more rapidly, with greater duration and/or with greater affinity with a particular cell, substance, or particular epitope than it does with alternative cells or substances, or epitopes. An antibody “specifically binds” or “preferentially binds” to a target molecule or epitope if it binds with greater affinity, avidity, more readily, and/or with greater duration than it binds to other substances or epitopes, for example, by a statistically significant amount. Typically one member of the pair of molecules that exhibit specific binding has an area on its surface, or a cavity, which specifically binds to and is therefore complementary to a particular spatial and/or polar organization of the other member of the pair of molecules. Thus, the members of the pair have the property of binding specifically to each other. For instance, an antibody that specifically or preferentially binds to a specific epitope is an antibody that binds that specific epitope with greater affinity, avidity, more readily, and/or with greater duration than it binds to other epitopes. It is also understood by reading this definition that, for example, an antibody (or moiety or epitope) that specifically or preferentially binds to a first target may or may not specifically or preferentially bind to a second target. The term is also applicable where, for example, an antibody is specific for a particular epitope which is carried by a number of antigens, in which case the specific binding member carrying the antigen-binding fragment or domain will be able to bind to the various antigens carrying the epitope; for example, it may be cross reactive to a number of different forms of a target antigen from multiple species that share a common epitope

Immunological binding generally refers to the non-covalent interactions of the type which occur between an immunoglobulin molecule and an antigen for which the immunoglobulin is specific, for example by way of illustration and not limitation, as a result of electrostatic, ionic, hydrophilic and/or hydrophobic attractions or repulsion, steric forces, hydrogen bonding, van der Waals forces, and other interactions. The strength, or affinity of immunological binding interactions can be expressed in terms of the dissociation constant (Kd) of the interaction, wherein a smaller Kd represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and on geometric parameters that equally influence the rate in both directions. Thus, both the “on rate constant” (Kon) and the “off rate constant” (Koff) can be determined by calculation of the concentrations and the actual rates of association and dissociation. The ratio of Koff/Kon enables cancellation of all parameters not related to affinity, and is thus equal to the dissociation constant Kd. As used herein, the term “affinity” includes the equilibrium constant for the reversible binding of two agents and is expressed as Kd or EC₅₀. Affinity of an antibody for a PD-1 or PD-L1 protein or epitope can be, for example, from about 100 nanomolar (nM) to about 0.1 nM, from about 100 nM to about 1 picomolar (pM), or from about 100 nM to about 1 femtomolar (fM). As used herein, the term “avidity” refers to the resistance of a complex of two or more agents to dissociation after dilution.

Programmed cell death protein 1 (PD-1; CD279 (cluster of differentiation 279)) refers a protein expressed on the surface of cells that regulates the immune response to the cells of the human body, for example, by down-regulating the immune system and promoting self-tolerance by suppressing T cell inflammatory activity (see Uniprot: Q15116). PD-1 is an immune checkpoint that promotes apoptosis of antigen-specific T-cells in lymph nodes, and reduces apoptosis in regulatory T cells (anti-inflammatory, suppressive T cells). Thus, in certain embodiments, the Fab region specifically binds to the human PD-1 protein sequence described in Uniprot: Q15116. Anti-PD-1 antibodies are known in the art (see, e.g., U.S. Pat. Nos. 8,008,449; 8,993,731; 9,073,994; 9,084,776; 9,102,727; 9,102,728; 9,181,342; 9,217,034; 9,387,247; 9,492,539; 9,492,540; and U.S. Application Nos. 2012/0039906; 2015/0203579). For example, in specific embodiments, the Fab region is from an anti-PD-1 antibody selected from nivolumab, pembrolizumab, cemiplimab, JTX-4014, spartalizumab, camrelizumab, sintilimab, tislelizumab, toripalimab, dostarlimab, MGA012, AMP-22, and AMP-514.

Programmed death-ligand 1 (PD-L1) is a 40 kDa type 1 transmembrane protein that binds to its receptor, PD-1, which is expressed on activated T cells, B cells, and myeloid cells, to modulate activation or inhibition (see Uniprot: Q9NZQ7). In certain embodiments, the Fab region specifically binds to the human PD-L1 protein sequence described in Uniprot: Q9NZQ7. Anti-PD-L1 antibodies are known in the art (see, e.g., U.S. Pat. Nos. 9,102,725; 9,393,301; 9,402,899; 9,439,962). For example, in specific embodiments, the Fab region is from an anti-PD-L1 antibody selected from atezolizumab, avelumab, and durvalumab.

In certain embodiments, an anti-PD-1 or anti-PD-L1 Fab region is characterized by or comprises a heavy chain variable region (VH) sequence that comprises complementary determining region VHCDR1, VHCDR2, and VHCDR3 sequences, and a light chain variable region (VL) sequence that comprises complementary determining region VLCDR1, VLCDR2, and VLCDR3 sequences. Exemplary VH, VHCDR1, VHCDR2, VHCDR3, VL, VLCDR1, VLCDR2, and VLCDR3 sequences are provided in Table P1 and Table P2 below.

TABLE P1
Exemplary anti-PD-1 antibodies

| | | |
|---|---|---|
| Name | Sequence | SEQ ID NO: |

Anti-PD-1 Antibody 1

| | | |
|---|---|---|
| VH | QVQLVQSGVEVKKPGASVKVSCKASGYTFTNYYMYWVRQAPGQGLEWMGGINPS | 1 |
| | NGGTNFNEKFKNRVTLTTDSSTTTAYMELKSLQFDDTAVYYCARRDYRFDMGFD | |
| | YWGQGTTVTVSS | |
| VL | EIVLTQSPATLSLSPGERATLSCRASKGVSTSGYSYLHWYQQKPGQAPRLLIYL | 2 |
| | ASYLESGVPARFSGSGSGTDFTLTISSLEPEDFAVYYCQHSRDLPLTFGGGTKV | |
| | EIK | |

Anti-PD-1 Antibody 2

| | | |
|---|---|---|
| VH | QVQLVESGGGVVQPGRSLRLDCKASGITFSNSGMHWVRQAPGKGLEWVAVIWYD | 3 |
| | GSKRYYADSVKGRFTISRDNSKNTLFLQMNSLRAEDTAVYYCATNDDYWGQGTL | |
| | VTVSS | |
| VL | EIVLTQSPATLSLSPGERATLSCRASQSVSSYLAWYQQKPGQAPRLLIYDASNR | 4 |
| | ATGIPARFSGSGSGTDFTLTISSLEPEDFAVYYCQQSSNWPRTFGQGTKVEIK | |

Anti-PD-1 Antibody 3

| | | |
|---|---|---|
| VH | QVQLQESGPGLVKPSETLSLTCTVSGFSLTSYGVHWIRQPPGKGLEWIGVIYAD | 5 |
| | GSTNYNPSLKSRVTISKDTSKNQVSLKLSSVTAADTAVYYCARAYGNYWYIDVW | |
| | GQGTTVTVSS | |
| VL | DIVMTQSPDSLAVSLGERATINCKSSESVSNDVAWYQQKPGQPPKLLINYAFHR | 6 |
| | FTGVPDRFSGSGYGTDFTLTISSLQAEDVAVYYCHQAYSSPYTFGQGTKLEIK | |

Anti-PD-1 Antibody 4

| | | |
|---|---|---|
| VH | QVQLVQSGAEVKKPGSSVKVSCKASGFTFTTYYISWVRQAPGQGLEYLGYINMG | 7 |
| | SGGTNYNEKFKGRVTITADKSTSTAYMELSSLRSEDTAVYYCAIIGYFDYWGQG | |
| | TMVTVSS | |
| VL | DVVMTQSPLSLPVTLGQPASISCRSSQSLLDSDGGTYLYWFQQRPGQSPRRLIY | 8 |
| | LVSTLGSGVPDRFSGSGSGTDFTLKISRVEAEDVGVYYCMQLTHWPYTFGQGTK | |
| | LEIK | |

Anti-PD-1 Antibody 5

| | | |
|---|---|---|
| VH | QGQLVQSGAEVKKPGASVKVSCKASGYTFTDYEMHWVRQAPIHGLEWIGVIESE | 9 |
| | TGGTAYNQKFKGRVTITADKSTSTAYMELSSLRSEDTAVYYCAREGITTVATTY | |
| | YWYFDVWGQGTTVTVSS | |
| VL | DVVMTQSPLSLPVTLGQPASISCRSSQSIVHSNGNTYLEWYLQKPGQSPQLLIY | 10 |
| | KVSNRFSGVPDRFSGSGSGTDFTLKISRVEAEDVGVYYCFQGSHVPLTFGQGTK | |
| | LEIK | |

Anti-PD-1 Antibody 6

| | | |
|---|---|---|
| VH | EVQLVESGGGLVQPGGSLRLSCAASGFTFSSYMMSWVRQAPGKGLEWVATISGG | 11 |
| | GANTYYPDSVKGRFTISRDNAKNSLYLQMNSLRAEDTAVYYCARQLYYFDYWGQ | |
| | GTTVTVSS | |
| VL | DIQMTQSPSSLSASVGDRVTITCLASQTIGTWLTWYQQKPGKAPKLLIYTATSL | 12 |
| | ADGVPSRFSGSGSGTDFTLTISSLQPEDFATYYCQQVYSIPWTFGGGTKVEIK | |

Anti-PD-1 Antibody 7

| | | |
|---|---|---|
| VH | QVQLVQSGAEVKKPGSSVKVSCKASGGTFSSYAISWVRQAPGQGLEWMGLIIPM | 13 |
| | FDTAGYAQKFQGRVAITVDESTSTAYMELSSLRSEDTAVYYCARAEHSSTGTFD | |
| | YWGQGTLVTVSS | |
| VL | DIQMTQSPSSVSASVGDRVTITCRASQGISSWLAWYQQKPGKAPKLLISAASSL | 14 |
| | QSGVPSRFSGSGSGTDFTLTISSLQPEDFATYYCQQANHLPFTFGGGTKVEIK | |

Anti-PD-1 Antibody 8

| | | |
|---|---|---|
| VH | QLQLQESGPGLVKPSETLTLTCTVSADSISSTTYYWVWIRQPPGKGLEWIGSIS | 15 |
| | YSGSTYYNPSLKSRVTVSVDTSKNQFSLKLNSVAATDTALYYCARHLGYNGRYL | |
| | PFDYWGQGTLVTVSS | |
| VL | QSALTQPASVSGSPGQSITISCTGTSSDVGFYNYVSWYQQHPGKAPELMIYDVS | 16 |
| | NRPSGVSDRFSGSKSGNTASLTISGLQAEDEADYYCSSYTSISTWVFGGGTKLT | |
| | VL | |

Anti-PD-1 Antibody 9

| | | |
|---|---|---|
| VH | EVQLVESGGGLVQPGGSLRLSCAASGFAFSSYDMSWVRQAPGKGLDWVATISGG | 17 |
| | GRYTYYPDSVKGRFTISRDNSKNNLYLQMNSLRAEDTALYYCANRYGEAWFAYW | |
| | GQGTLVTVSS | |
| VL | DIQMTQSPSSMSASVGDRVTFTCRASQDINTYLSWFQQKPGKSPKTLIYRANRL | 18 |
| | VSGVPSRFSGSGSGQDYTLTISSLQPEDMATYYCLQYDEFPLTFGAGTKLELK | |

Anti-PD-1 Antibody 10

| | | |
|---|---|---|
| VH | EVQLVQSGGGLVQPGGSLKLSCAASGFTFSSYGMSWVRQAPGKGLDWVATISGG | 19 |
| | GRDTYYPDSVKGRFTISRDNSKNNLYLQMNSLRAEDTALYYCARQKGEAWFAYW | |
| | GQGTLVTVSS | |
| VL | DIVLTQSPASLAVSPGQRATITCRASESVDNYGISFMNWFQQKPGQPPKLLIYA | 20 |
| | ASNKGTGVPARFSGSGSGTDFTLNINPMEENDTAMYFCQQSKEVPWTFGGGTKL | |
| | EIK | |

Anti-PD-1 Antibody 11

| | | |
|---|---|---|
| VH | QVQLVESGGGLVKPGGSLRLSCAASGFTFSNYGMSWIRQAPGKGLEWVSTISGG | 21 |
| | GSNIYYADSVKGRFTISRDNAKNSLYLQMNSLRAEDTAVYYCVSYYYGIDFWGQ | |
| | GTSVTVSS | |
| VL | DIQMTQSPSSLSASVGDRVTITCKASQDVTTAVAWYQQKPGKAPKLLIYWASTR | 22 |
| | HTGVPSRFSGSGSGTDFTLTISSLQPEDFATYYCQQHYTIPWTFGGGTKLEIK | |

Anti-PD-1 Antibody 12

| | | |
|---|---|---|
| VH | QIQLVQSGSELKKPGASVKVSCKASGYTFTNFGMNWVRQAPGQGLKWMGWISGY | 23 |
| | TREPTYAADFKGRFVISLDTSVSTAYLQISSLKAEDTAVYYCARDVFDYWGQGT | |
| | LVTVSS | |
| VI | DIVLTQSPASLAVSPGQRATITCRASESVDNYGYSFMNWFQQKPGQPPKLLIYR | 24 |
| | ASNLESGVPARFSGSGSRTDFTLTINPVEADDTANYYCQQSNADPTFGQGTKLE | |
| | IK | |

TABLE P2
Exemplary anti-PD-L1 antibodies

| | | |
|---|---|---|
| Name | Sequence | SEQ ID NO: |

Anti-PD-L1 Antibody 1

| | | |
|---|---|---|
| VH | QVQLVQSGAEVKKPGSSVKVSCKTSGDTFSTYAISWVRQAPGQGLEWMGGIIPI | 25 |
| | FGKAHYAQKFQGRVTITADESTSTAYMELSSLRSEDTAVYFCARKFHFVSGSPF | |
| | GMDVWGQGTTVTVSS | |
| VL | EIVLTQSPATLSLSPGERATLSCRASQSVSSYLAWYQQKPGQAPRLLIYDASNR | 26 |
| | ATGIPARFSGSGSGTDFTLTISSLEPEDFAVYYCQQRSNWPTFGQGTKVEIK | |

Anti-PD-L1 Antibody 2

| | | |
|---|---|---|
| VH | EVQLVESGGGLVQPGGSLRLSCAASGFTFSDSWIHWVRQAPGKGLEWVAWISPY | 27 |
| | GGSTYYADSVKGRFTISADTSKNTAYLQMNSLRAEDTAVYYCARRHWPGGFDYW | |
| | GQGTLVTVSS | |
| VL | DIQMTQSPSSLSASVGDRVTITCRASQDVSTAVAWYQQKPGKAPKLLIYSASFL | 28 |
| | YSGVPSRFSGSGSGTDFTLTISSLQPEDFATYYCQQYLYHPATFGQGTKVEIK | |

Anti-PD-L1 Antibody 3

| | | |
|---|---|---|
| VH | EVQLVESGGGLVQPGGSLRLSCAASGFTFSRYWMSWVRQAPGKGLEWVANIKQD | 29 |
| | GSEKYYVDSVKGRFTISRDNAKNSLYLQMNSLRAEDTAVYYCAREGGWFGELAF | |
| | DYWGQGTLVTVSS | |
| VL | EIVLTQSPGTLSLSPGERATLSCRASQRVSSSYLAWYQQKPGQAPRLLIYDASS | 30 |
| | RATGIPDRFSGSGSGTDFTLTISRLEPEDFAVYYCQQYGSLPWTFGQGTKVEIK | |

Anti-PD-L1 Antibody 4

| | | |
|---|---|---|
| VH | QVQLVQSGAEVKKPGASVKLSCKASGYIFTTYWMHWVKQRPGQGLEWIGMIQPN | 31 |
| | SGGTKYNEKFKKKATLTVDKSISTAYMELSRLTSDDTAVYYCARGAGTVDYFDY | |
| | WGQGSTLTVSS | |
| VL | DIVLTQSPASLAVSVGQRATITCRASESVDIYGNSFMHWYQQKPGQPPKLLIYR | 32 |
| | ASNLESGIPARFSGSGSRTDFTLTINPVEAQDTATYYCQQSTEDPYTFGGGTKL | |
| | EIK | |

Anti-PD-L1 Antibody 5

| | | |
|---|---|---|
| VH | EVQLLESGGGLVQPGGSLRLSCAASGFTFSSYAMSWVRQAPGKGLEWVSGISGS | 33 |
| | GGFTYYADSVKGRFTISRDNSKNTLYLQMNSLRAEDTAVYYCAKPPRGYNYGPF | |
| | DYWGQGTLVTVSS | |
| VL | SYVLTQPPSVSVAPGQTARITCGGNNIGSKSVHWYQQKPGQAPVLVVYDDSDRP | 34 |
| | SGIPERFSGSNSGNTATLTISRVEAGDEADYYCQVWDSSSDHVVFGGGTKLTVL | |

Anti-PD-L1 Antibody 6

| | | |
|---|---|---|
| VH | EVQLVESGAEVKKPGSSVKVSCKASGGTFSSYAISWVRQAPGQGLEWMGGIIPI | 35 |
| | FGTANYAQKFQGRVTITADESTSTAYMELSSLRSEDTAVYYCARAPYYYYYMDV | |
| | WGQGTTVTVSS | |
| VL | QSALTQPASVSGSLGQSVTISCTGSSSDVGSYNLVSWYQQHPGKAPNLMIYDVS | 36 |
| | KRSGVSNRFSGSKSGNTASLTISGLQAEDEADYYCSSYTGISTVVFGGGTKLTV | |
| | L | |

Anti-PD-L1 Antibody 7

| | | |
|---|---|---|
| VH | QVQLQESGPGLVKPSETLSITCTVSGFSLSNYDISWIRQPPGKGLEWLGVIWTG | 37 |
| | GATNYNPALKSRLTISRDNSKNQVSLKMSSVTAADTAVYYCVRDSNYRYDEPFT | |
| | YWGQGTLVTVSS | |
| VL | EIVLTQSPDTLSVTPKEKVTLTCRASQSIGTNIHWFQQKPGQSPKLLIKYASES | 38 |
| | ISGVPSRFSGSGSGTDFTLTINSVEAEDAATYYCQQSNSWPYTFGQGTKLEIK | |

Thus, in certain embodiments, an anti-PD-1 Fab region thereof comprises a heavy chain variable (VH) region comprising VHCDR1, VHCDR2, and VHCDR3 regions (underlined) from Table P1, and a corresponding light chain variable (VL) region comprising the VLCDR1, VLCDR2, and VLCDR3 regions (underlined) from Table P1. Also included are variants thereof that bind to human PD-1, for example, variants having 1, 2, 3, 4, 5, or 6 total alterations in the combined CDR regions, for example, the VHCDR1, VHCDR2, VHCDR3, VLCDR1, VLCDR2, and/or VLCDR3 sequences described herein. Exemplary “alterations” include amino acid substitutions, additions, and deletions. In certain embodiments, an anti-PD-1 Fab region comprises a VH region from Table P1, and the corresponding VL region from Table P1. In certain embodiments, the VH region comprises an amino acid sequence having at least 90%, 95%, 96%, 97%, 98%, 99%, or 100% identity to a sequence selected from Table P1, and the VL region comprises an amino acid sequence having at least 90%, 95%, 96%, 97%, 98%, 99%, or 100% identity to the corresponding sequence selected Table P1. Also included are variants thereof that bind to human PD-1, for example, variants having 1, 2, 3, 4, 5, 6 alterations in one or more framework regions. Exemplary “alterations” include amino acid substitutions, additions, and deletions.

Thus, in certain embodiments, an anti-PD-L1 Fab region thereof comprises a heavy chain variable (VH) region comprising VHCDR1, VHCDR2, and VHCDR3 regions (underlined) from Table P2, and a corresponding light chain variable (VL) region comprising the VLCDR1, VLCDR2, and VLCDR3 regions (underlined) from Table P2. Also included are variants thereof that bind to human PD-L1, for example, variants having 1, 2, 3, 4, 5, or 6 total alterations in the combined CDR regions, for example, the VHCDR1, VHCDR2, VHCDR3, VLCDR1, VLCDR2, and/or VLCDR3 sequences described herein. Exemplary “alterations” include amino acid substitutions, additions, and deletions. In certain embodiments, an anti-PD-L1 Fab region comprises a VH region from Table P2, and the corresponding VL region from Table P2. In certain embodiments, the VH region comprises an amino acid sequence having at least 90%, 95%, 96%, 97%, 98%, 99%, or 100% identity to a sequence selected from Table P2, and the VL region comprises an amino acid sequence having at least 90%, 95%, 96%, 97%, 98%, 99%, or 100% identity to the corresponding sequence selected Table P2. Also included are variants thereof that bind to human PD-L1, for example, variants having 1, 2, 3, 4, 5, 6 alterations in one or more framework regions. Exemplary “alterations” include amino acid substitutions, additions, and deletions.

CD276 (B7H3) is an immune checkpoint molecule that participates in the regulation of T-cell-mediated immune responses, and is expressed on some solid tumors. It plays a protective role in tumor cells, for example, by inhibiting natural-killer mediated cell lysis and potentially other anti-tumor immune responses. In particular embodiments, the B7H3 is human B7H3, or a domain thereof.

In certain embodiments, an anti-B7H3 Fab region specifically binds to a human B7H3 protein, for example, a domain of human B7H3 selected from one or more of the Ig-like V-type 1 domain, Ig-like C2-type 1 domain, Ig-like V-type 2 domain, and an Ig-like C2-type 2 domain. In specific embodiments, a Fab region specifically binds to human BH73 with a K_{D} of about 0.4 or 0.5 nM (400 or 500 pM) or lower.

In certain embodiments, an anti-B7H3 Fab region is characterized by or comprises a VH sequence that comprises complementary determining region VHCDR1, VHCDR2, and VHCDR3 sequences, and a VL sequence that comprises complementary determining region VLCDR1, VLCDR2, and VLCDR3 sequences. Exemplary VH, VHCDR1, VHCDR2, VHCDR3, VL, VLCDR1, VLCDR2, and VLCDR3 sequences are provided in Table P3 below.

TABLE P3
Exemplary anti-B7H3 antibodies

| | | |
|---|---|---|
| Name | Sequence | SEQ ID NO: |

Anti-B7H3 Antibody 1

| | | |
|---|---|---|
| VH | QVQLVQSGSELKKPGASVKVSCKASGYTFTNYDINWVRQAPGQGLEWMGWIFPG | 202 |
| | DESTQYNEKFKGRFVFSLDTSVSTAYLQISSLKAEDTAVYYCARQTTATWFAYW | |
| | GQGTLVTVSS | |
| VL | DVVMTQSPAFLSVTPGEKVTITCRASQSISDYLHWYQQKPDQAPKLLIKYASQS | 203 |
| | ISGIPARFSGSGSGTDFTFTISSLEAEDAATYYCQNGHSFPLTFGQGTKLELK | |

Thus, in certain embodiments, an anti-B7H3 Fab region thereof comprises a VH region comprising VHCDR1, VHCDR2, and VHCDR3 regions (underlined) from Table P3, and a corresponding VL region comprising the VLCDR1, VLCDR2, and VLCDR3 regions (underlined) from Table P3. Also included are variants thereof that bind to human B7H3, for example, variants having 1, 2, 3, 4, 5, or 6 total alterations in the combined CDR regions, for example, the VHCDR1, VHCDR2, VHCDR3, VLCDR1, VLCDR2, and/or VLCDR3 sequences described herein. Exemplary “alterations” include amino acid substitutions, additions, and deletions. In certain embodiments, an anti-B7H3 Fab region comprises a VH region from Table P3, and the corresponding VL region from Table P3. In certain embodiments, the VH region comprises an amino acid sequence having at least 90%, 95%, 96%, 97%, 98%, 99%, or 100% identity to a sequence selected from Table P3, and the VL region comprises an amino acid sequence having at least 90%, 95%, 96%, 97%, 98%, 99%, or 100% identity to the corresponding sequence selected Table P3. Also included are variants thereof that bind to human B7H3, for example, variants having 1, 2, 3, 4, 5, 6 alterations in one or more framework regions. Exemplary “alterations” include amino acid substitutions, additions, and deletions.

Antibodies or Fab regions may be prepared by any of a variety of techniques known to those of ordinary skill in the art. See, e.g., Harlow and Lane, Antibodies: A Laboratory Manual, Cold Spring Harbor Laboratory, 1988. Monoclonal antibodies specific for a polypeptide of interest may be prepared, for example, using the technique of Kohler and Milstein, Eur. J. Immunol. 6:511-519, 1976, and improvements thereto. Also included are methods that utilize transgenic animals such as mice to express human antibodies or Fab regions. See, e.g., Neuberger et al., Nature Biotechnology 14:826, 1996; Lonberg et al., Handbook of Experimental Pharmacology 113:49-101, 1994; and Lonberg et al., Internal Review of Immunology 13:65-93, 1995. The structures and locations of immunoglobulin variable domains may be determined by reference to Kabat, E. A. et al., Sequences of Proteins of Immunological Interest. 4th Edition. US Department of Health and Human Services. 1987, and updates thereof.

It will be appreciated that any one or more of the foregoing anti-PD-1 Fabs or anti-PD-L1 Fabs can be combined with any of the other components described herein, for example, hinge/Fc domains, IL-15 proteins, IL-15Rα proteins, and linkers described herein, to generate one or more activatable proproteins.

Hinge/Fc Domains. Certain activatable proprotein homodimers comprises a hinge/Fc domain. The hinge region (found in IgG, IgA, and IgD) acts as a flexible spacer that allows the Fab portion to move freely in space relative to the Fc domain. In contrast to the constant regions, the hinge regions are structurally diverse, varying in both sequence and length among immunoglobulin classes and subclasses. The hinge region may also contain one or more glycosylation site(s), which include a number of structurally distinct types of sites for carbohydrate attachment. For example, IgA1 contains five glycosylation sites within a 17 amino acid segment of the hinge region, conferring significant resistance of the hinge region polypeptide to intestinal proteases. Residues in the hinge proximal region of the CH₂ domain can also influence the specificity of the interaction between an immunoglobulin and its respective Fc receptor(s) (see, e.g., Shin et al., Intern. Rev. Immunol. 10:177-186, 1993).

The term “Fc domain” or “Fc fragment” or “Fc” refers to a protein that contains one or more of a CH2 domain, a CH3 domain, and/or a CH4 domain from one or more selected immunoglobulin(s), including fragments and variants and combinations thereof. An “Fc domain” may also include one or more hinge region(s) of the heavy chain constant region of an immunoglobulin. In certain embodiments, the Fc domain does not contain one or more of the CH1, CL, VL, and/or VH regions of an immunoglobulin.

The Fc domain can be derived from the CH2 domain, CH3 domain, CH4 domain, and/or hinge region(s) of any one or more immunoglobulin classes, including but not limited to IgA, IgD, IgE, IgG, IgM, including subclasses and combinations thereof. In some embodiments, the Fc domain is derived from an IgA immunoglobulin, including subclasses IgA1 and/or IgA2. In certain embodiments, the Fc domain is derived from an IgD immunoglobulin. In particular embodiments, the Fc domain is derived from an IgE immunoglobulin. In some embodiments, the Fc domain is derived from an IgG immunoglobulin, including subclasses IgG1, IgG2, IgG2, IgG3, and/or IgG4. In certain embodiments, the Fc domain is derived from an IgM immunoglobulin. Exemplary hinge and Fc domain sequences are provided in Table PR below.

TABLE F1
Exemplary Hinge and Fc Domain Sequences

| Name | Sequence | SEQ ID NO: |
|---|---|---|
| IgA1 hinge | VPSTPPTPSPSTPPTPSPS | 39 |
| IgA2 hinge | VPPPPP | 40 |
| IgD hinge | ESPKAQASSVPTAQPQAEGSLAKATTAPATTRNTGRGGEEKKKEKEKEEQE | 41 |
| | ERETKTP | |
| IgG1 hinge | EPKSCDKTHTCPPCP | 42 |
| modified | SDKTHTCPPCP | 43 |
| human IgG1 | | |
| hinge | | |
| IgG2 hinge | ERKCCVECPPCP | 44 |
| human IgG3 | ELKTPLGDTTHTCPRCPEPKSCDTPPPCPRCPEPKSCDTPPPCPRCPEPKS | 45 |
| hinge | CDTPPPCPRCP | |
| IgG4 hinge | ESKYGPPCPSCP | 46 |
| IgA1 CH2 | CCHPRLSLHRPALEDLLLGSEANLTCTLTGLRDASGVTFTWTPSSGKSAVQ | 47 |
| domain | GPPERDLCGCYSVSSVLPGCAEPWNHGKTFTCTAAYPESKTPLTATLSKS | |
| IgA1 CH3 | GNTFRPEVHLLPPPSEELALNELVTLTCLARGFSPKDVLVRWLQGSQELPR | 48 |
| domain | EKYLTWASRQEPSQGTTTFAVTSILRVAAEDWKKGDTFSCMVGHEALPLAF | |
| | TQKTIDRLAGKPTHVNVSVVMAEVDGTCY | |
| IgA2 CH2 | CCHPRLSLHRPALEDLLLGSEANLTCTLTGLRDASGATFTWTPSSGKSAVQ | 49 |
| domain | GPPERDLCGCYSVSSVLPGCAQPWNHGETFTCTAAHPELKTPLTANITKS | |
| IgA2 CH3 | GNTFRPEVHLLPPPSEELALNELVTLTCLARGFSPKDVLVRWLQGSQELPR | 50 |
| domain | EKYLTWASRQEPSQGTTTFAVTSILRVAAEDWKKGDTFSCMVGHEALPLAF | |
| | TQKTIDRLAGKPTHVNVSVVMAEVDGTCY | |
| IgD CH2 | ECPSHTQPLGVYLLTPAVQDLWLRDKATFTCFVVGSDLKDAHLTWEVAGKV | 51 |
| domain | PTGGVEEGLLERHSNGSQSQHSRLTLPRSLWNAGTSVTCTLNHPSLPPQRL | |
| | MALREP | |
| IgD CH3 | AAQAPVKLSLNLLASSDPPEAASWLLCEVSGFSPPNILLMWLEDQREVNTS | 52 |
| | GFAPARPPPQPRSTTFWAWSVLRVPAPPSPQPATYTCVVSHEDSRTLLNAS | |
| | RSLEVSYVTDHGPMK | |
| IgE CH2 | VCSRDFTPPTVKILQSSCDGGGHFPPTIQLLCLVSGYTPGTINITWLEDGQ | 53 |
| domain | VMDVDLSTASTTQEGELASTQSELTLSQKHWLSDRTYTCQVTYQGHTFEDS | |
| | TKKCA | |
| IgE CH3 | DSNPRGVSAYLSRPSPFDLFIRKSPTITCLVVDLAPSKGTVNLTWSRASGK | 54 |
| domain | PVNHSTRKEEKQRNGTLTVTSTLPVGTRDWIEGETYQCRVTHPHLPRALMR | |
| | STTKTS | |
| IgE CH4 | GPRAAPEVYAFATPEWPGSRDKRTLACLIQNEMPEDISVQWLHNEVQLPDA | 55 |
| domain | RHSTTQPRKTKGSGFFVFSRLEVTRAEWEQKDEFICRAVHEAASPSQTVQR | |
| | AVSVNPGK | |
| IgG1 CH2 | APELLGGPSVFLFPPKPKDTLMISRTPEVTCVVVDVSHEDPEVKENWYVDG | 56 |
| domain | VEVHNAKTKPREEQYNSTYRVVSVLTVLHQDWLNGKEYKCKVSNKALPAPI | |
| | EKTISKAK | |
| Modified | APEAAGGPSVFLFPPKPKDTLMISRTPEVTCVVVDVSHEDPEVKENWYVDG | 57 |
| IgG1 CH2 | VEVHNAKTKPREEQYNSTYRVVSVLTVLHQDWLNGKEYKCKVSNKALAAPI | |
| domain | EKTISKAK | |
| LALA-P329A | | |
| IgG1 CH3 | GQPREPQVYTLPPSRDELTKNQVSLTCLVKGFYPSDIAVEWESNGQPENNY | 58 |
| domain | KTTPPVLDSDGSFFLYSKLTVDKSRWQQGNVFSCSVMHEALHNHYTQKSLS | |
| | LSPG | |
| IgG2 CH2 | APPVAGPSVFLFPPKPKDTLMISRTPEVTCVVVDVSHEDPEVQFNWYVDGV | 59 |
| domain | EVHNAKTKPREEQFNSTFRVVSVLTVVHQDWLNGKEYKCKVSNKGLPAPIE | |
| | KTISKTK | |
| IgG2 CH3 | GQPREPQVYTLPPSREEMTKNQVSLTCLVKGFYPSDIAVEWESNGQPENNY | 60 |
| domain | KTTPPMLDSDGSFFLYSKLTVDKSRWQQGNVFSCSVMHEALHNHYTQKSLS | |
| | LSPGK | |
| IgG3 CH2 | APELLGGPSVFLFPPKPKDTLMISRTPEVTCVVVDVSHEDPEVQFKWYVDG | 61 |
| domain | VEVHNAKTKPREEQYNSTFRVVSVLTVLHQDWLNGKEYKCKVSNKALPAPI | |
| | EKTISKTK | |
| IgG3 CH3 | GQPREPQVYTLPPSREEMTKNQVSLTCLVKGFYPSDIAVEWESSGQPENNY | 62 |
| domain | NTTPPMLDSDGSFFLYSKLTVDKSRWQQGNIFSCSVMHEALHNRFTQKSLS | |
| | LSPGK | |
| IgG4 CH2 | APEFLGGPSVFLFPPKPKDTLMISRTPEVTCVVVDVSQEDPEVQFNWYVDG | 63 |
| domain | VEVHNAKTKPREEQFNSTYRVVSVLTVLHQDWLNGKEYKCKVSNKGLPSSI | |
| | EKTISKAK | |
| IgG4 CH3 | GQPREPQVYTLPPSQEEMTKNQVSLTCLVKGFYPSDIAVEWESNGQPENNY | 64 |
| domain | KTTPPVLDSDGSFFLYSRLTVDKSRWQEGNVFSCSVMHEALHNHYTQKSLS | |
| | LSLGK | |
| IgM CH2 | VIAELPPKVSVFVPPRDGFFGNPRKSKLICQATGFSPRQIQVSWLREGKQV | 65 |
| domain | GSGVTTDQVQAEAKESGPTTYKVTSTLTIKESDWLGQSMFTCRVDHRGLTF | |
| | QQNASSMCVP | |
| IgM CH3 | DQDTAIRVFAIPPSFASIFLTKSTKLTCLVTDLTTYDSVTISWTRQNGEAV | 66 |
| domain | KTHTNISESHPNATFSAVGEASICEDDWNSGERFTCTVTHTDLPSPLKQTI | |
| | SRPK | |
| IgM CH4 | GVALHRPDVYLLPPAREQLNLRESATITCLVTGFSPADVFVQWMQRGQPLS | 67 |
| domain | PEKYVTSAPMPEPQAPGRYFAHSILTVSEEEWNTGETYTCVVAHEALPNRV | |
| | TERTVDKSTGKPTLYNVSLVMSDTAGTCY | |

Thus, in some embodiments, the hinge comprises an amino acid sequence having at least 90%, 95%, 96%, 97%, 98%, 99%, or 100% identity to a hinge sequence selected from Table F1, for instance, an IgA1, IgA2, IgD, IgG1, IgG2, IgG3, IgG4 hinge region selected from Table F1. In certain embodiments, the Fc domain comprises an amino acid sequence having at least 90%. 95%, 96%, 97%, 98%, 99%, or 100% identity to a sequence selected from Table F1, for instance, an IgA1 CH2, CH3, or combined CH2CH3 sequence, an IgA2 CH2, CH3, or combined CH2CH3 sequence, an IgD CH2, CH3, or combined CH2CH3 sequence, an IgE CH2, CH3, CH4, or combined CH2CH3 or CH2CH3CH4 sequence, an IgG1 CH2, CH3, or combined CH2CH3 sequence, an IgG2 CH2, CH3, or combined CH2CH3 sequence, an IgG3 CH2, CH3, or combined CH2CH3 sequence, an IgG4 CH2, CH3, or combined CH2CH3 sequence, or an IgM CH2, CH3, CH4, or combined CH2CH3 or CH2CH3CH4 sequence from Table F1. In certain embodiments, the hinge is of the same Ig class as the Fc domain.

In certain embodiments, the Fc domain is a modified Fc domain. Such modifications can be employed to alter (e.g., increase, decrease) the binding properties of the Fc region to one or more particular FcRs (e.g., FcγRI, FcγRIIa, FcγRIIb, FcγRIIc, FcγRIIIa, FcγRIIIb, FcRn), its pharmacokinetic properties (e.g., stability or half-life, bioavailability, tissue distribution, volume of distribution, concentration, elimination rate constant, elimination rate, area under the curve (AUC), clearance, Cₘₐₓ, tₘₐₓ, Cₘᵢₙ, fluctuation), its immunogenicity, its complement fixation or activation, and/or the CDC/ADCC/ADCP-related activities of the Fc region, among other properties described herein, relative to a corresponding wild-type Fc sequence.

In some embodiments, the modified Fc domain does not bind or does not substantially bind to FcγR. Examples of FcγRs include FcγRI, FcγRIIa, FcγRIIb, FcγRIIc, FcγRIIIa, and FcγRIIIb. FcγRI (CD64) is expressed on macrophages and dendritic cells and plays a role in phagocytosis, respiratory burst, cytokine stimulation, and dendritic cell endocytic transport. Expression of FcγRI is upregulated by both GM-CSF and γ-interferon (γ-IFN) and downregulated by interleukin-4 (IL-4). FcγRIIa is expressed on polymorphonuclear leukocytes (PMN), macrophages, dendritic cells, and mast cells. FcγRIIa plays a role in phagocytosis, respiratory burst, and cytokine stimulation. Expression of FcγRIIa is upregulated by GM-CSF and γ-IFN, and decreased by IL-4. FcγIIb is expressed on B cells, PMN, macrophages, and mast cells. FcγIIb inhibits immunoreceptor tyrosine-based activation motif (ITAM) mediated responses, and is thus an inhibitory receptor. Expression of FcγRIIc is upregulated by intravenous immunoglobulin (IVIG) and IL-4 and decreased by γ-IFN. FcγRIIc is expressed on NK cells. FcγRIIIa is expressed on natural killer (NK) cells, macrophages, mast cells, and platelets. This receptor participates in phagocytosis, respiratory burst, cytokine stimulation, platelet aggregation and degranulation, and NK-mediated ADCC. Expression of FcγRIII is upregulated by C5a, TGF-β, and γ-IFN and downregulated by IL-4. Fc γ RIIIb is a GPI-linked receptor expressed on PMN.

In specific embodiments, the modified Fc domain comprises the L234A/L235A (“LALA”) mutations, and/or the P329A or P329G mutations (EU numbering) (see, for example, CH2 domain of SEQ ID NO: 57). In certain embodiments, the Fc domain or modified Fc domain retains normal (wild-type) or substantially normal binding to the neonatal Fc receptor (FcRn). In specific embodiments, the Fc region comprises the IgG1 hinge region of SEQ ID NO: 42, the modified IgG1 CH2 domain of SEQ ID NO: 57, and the IgG1 CH domain of SEQ ID NO: 58.

It will be appreciated that any one or more of the foregoing hinge and Fc domains can be combined with any of the other components described herein, for example, anti-PD-1 Fabs, anti-PD-L1 Fabs, IL-15 proteins, IL-15Rα proteins, and linkers described herein, to generate one or more activatable proproteins.

IL-15 Proteins. The activatable proproteins described herein comprise at least one “IL-15 protein” (or Interleukin-15 protein), including human IL-15 proteins. IL-15 is a pleiotropic cytokine that has been shown to induce and regulate a myriad of immune functions. For example, IL-15 is critical for lymphoid development, peripheral maintenance of innate immune cells, and immunological memory of T cells, mainly natural killer (NK) and CD8+ T cell populations. Like IL-2, IL-15 binds to and signals through a complex composed of IL-2/IL-15 receptor beta chain (CD122) and the common gamma chain (gamma-C, CD132).

IL-15 is a 14-15 kDA protein composed of a signal peptide (residues 1-29), a propeptide (residues 30-48), and an active mature protein (residues 49-162). Exemplary IL-15 protein sequences are provided in Table S1.

TABLE S1
Exemplary IL-15 Proteins

| Name | Sequence | SEQ ID NO: |
|---|---|---|
| Human IL-15 | MRISKPHLRSISIQCYLCLLLNSHFLTEAGIHVFILGCFSAGLPKTEANW | 68 |
| FL precursor | VNVISDLKKIEDLIQSMHIDATLYTESDVHPSCKVTAMKCFLLELQVISL | |
| | ESGDASIHDTVENLIILANNSLSSNGNVTESGCKECEELEEKNIKEFLQS | |
| | FVHIVQMFINTS | |
| Human IL-15 | NWVNVISDLKKIEDLIQSMHIDATLYTESDVHPSCKVTAMKCFLLELQVI | 69 |
| mature form | SLESGDASIHDTVENLIILANNSLSSNGNVTESGCKECEELEEKNIKEFL | |
| | QSFVHIVQMFINTS | |
| Human IL-15 | NWVNVISDLKKIEDLIQSMHIDATLYTESDVHPSCKVTAMKCFLLELQVI | 70 |
| mature form | SLESGDASIHDTVENLIILANNSLSSNGNVTESGCKECEELEEKNIKEFL | |
| with S162A | QSFVHIVQMFINTA | |
| Human IL-15 | NWVNVISDLKKIEDLIQSMHIDATLYTESDVHPSCKVTAMKCFLLELQDI | 71 |
| mature form | SLESGDASIHDTVENLIILANNSLSSNGNVTESGCKECEELEEKNIKEFL | |
| with V49D, | QSFVHIVQMFINTA | |
| S162A | | |
| Human IL-15 | NWVNVISDLKKIEDLIQSMHIDATLYTESDVHPSCKVTAMKCFLLELQVD | 72 |
| mature form | SLESGDASIHDTVENLIILANNSLSSNGNVTESGCKECEELEEKNIKEFL | |
| with I50D, | QSFVHIVQMFINTA | |
| S162A | | |
| Human IL-15 | NWVNVISDLKKIEDLIQSMHIDATLYTESDVHPSCKVTAMKCFLLELQVI | 73 |
| mature form | SLESGDASIHDTVENEIILANNSLSSNGNVTESGCKECEELEEKNIKEFL | |
| with L66E, | QSFVHIVQMFINTA | |
| S162A | | |
| Human IL-15 | NWVNVISNLKKIEDLIQSMHIDATLYTESDVHPSCKVTAMKCFLLELQVI | 74 |
| mature form | SLESGDASIHDTVENLIILANNSLSSNGNVTESGCKECEELEEKNIKEFL | |
| with D8N, | QSFVHIVQMFINTA | |
| S162A | | |
| Human IL-15 | NWVNVISDLKKIEDLIQSMHIDATLYTESDVHPSCKVTAMKCFLLKLQVI | 75 |
| mature form | SLESGDASIHDTVENLIILANNSLSSNGNVTESGCKECEELEEKNIKEFL | |
| with E46K, | QSFVHIVQMFINTA | |
| S162A | | |
| Human IL-15 | NWVNVISDLKKIEDLIQSMHIDATLYTESDVHPSCKVTAMKCFLLKLQVI | 76 |
| mature form | SLKSGDASIHDTVENLIILANNSLSSNGNVTESGCKECEELEEKNIKEFL | |
| with E46K, | QSFVHIVQMFINTA | |
| E53K, S162A | | |
| Human IL-15 | NWVNVISDLKKIEDLIQSMHIKATLYTESDVHPSCKVTAMKCFLLKLQVI | 77 |
| mature form | SLESGDASIHDTVENLIILANNSLSSNGNVTESGCKECEELEEKNIKEFL | |
| with D22K, | QSFVHIVQMFINTA | |
| E46K, S162A | | |
| Human IL-15 | NWVNVISDLKKIEDLIQSMHIKATLYTESDVHPSCKVTAMKCELLKLQVI | 78 |
| mature form | SLKSGDASIHDTVENLIILANNSLSSNGNVTESGCKECEELEEKNIKEFL | |
| with D22K, | QSFVHIVQMFINTA | |
| E46K, E53K, | | |
| S162A | | |
| Human IL-15 | NWVNVISDLKKIEDLIQSMHIDATLYTESDVHPSCKVTAMKCFLLELQVI | 79 |
| mature form | SLESGDASIHDTVENLIILANNSLSSNGNVTESGCGECEELEEKNIKEFL | |
| with K86G | QSFVHIVQMFINTA | |
| and S162A | | |

Thus, in certain embodiments, an IL-15 protein comprises, consists, or consists essentially of an amino acid sequence selected from Table S1, or an active variant or fragment thereof that is at least 80, 85, 90, 95, 98, or 100% identical to a sequence selected from Table S1. In some embodiments, an “active” IL-15 protein or fragment or variant is characterized, for example, by its ability to bind to an IL-15β/γc and/or IL-15Rα/β/γc receptor chain present on the surface of an immune cell in vitro or in vivo, and stimulate downstream signaling activities, absent steric hindrance by the binding moieties described herein. Examples of downstream signaling activities include IL-15 mediated signaling via Janus kinase 1 (Jak1) and γc subunit Janus kinase 3 (Jak3), which leads to phosphorylation and activation of signal transducer and activator of transcription 3 (STAT3) and STAT5 pathways. Additional examples include activation of Src family kinases including Lek and Fyn, and subsequent activation of PI3K and MAPK signaling pathways. Altogether, IL-15 signaling stimulates an array of downstream pathways leading to responses that have a significant role in the regulating the activation and proliferation of T and natural killer (NK) cells, and the survival of memory T cells, among others.

In particular embodiments, the IL-15 protein is a mature form of IL-15, or an active variant or fragment thereof, which comprises, consists, or consists essentially of an amino acid sequence that is at least 80, 85, 90, 95, 98, or 100% identical to amino acids 49-162 of SEQ ID NO: 68 (Human IL-15 FL precursor). Certain IL-15 proteins comprise, consist, or consist essentially of an amino acid sequence that is at least 80, 85, 90, 95, 98, or 100% identical to SEQ ID NO: 69 (mature human IL-15).

Certain IL-15 proteins comprise one or more defined amino acid substitutions relative to the exemplary amino acid sequences in Table S1. For example, in certain embodiments an IL-15 protein comprises or retains one or more amino acid substitutions at position D8, D22, E46, V49, I50, L66, K86 as defined by SEQ ID NO: 69 (mature human IL-15), and/or S162 as defined by SEQ ID NO: 68 (IL-15 FL precursor). Specific examples of substitutions are selected from one or more of D8N, D22K, E46K, V49D, I50D, L66E, K86G, and 162A, including combinations thereof (see Table S1). Exemplary combinations of substitutions are selected from V49D and S162A; I50D and S162A; L66E and S162A; D8N and S162A; V49D and S162A; E46K and S162A; E46K, E53K, and S162A; D22K, E46K, and S162A; and D22K, E46K, E53K, and S162A. In specific embodiments, the IL-15 protein comprises K86G and S162A mutations, as defined by SEQ ID NO: 69, for example, the mature IL-15 protein with K86G and S162A mutations (e.g., SEQ ID NO: 79).

In some embodiments, a D8N substitution in IL-15 does not significantly reduce binding affinity to IL-15Rα significantly reduces or all but eliminates IL-15 signaling activity. In some embodiments a V49D substitution in IL-15 has significantly lower (e.g., about 13 fold lower) binding affinity to IL-15Rα and retains about or at least about 90-100% of IL-15 signaling activity. In some embodiments, an I50D substitution in IL-15 has significantly lower (e.g., about 100 fold lower) binding affinity to IL-15Rα and retains about 10% of IL-15 signaling activity. In some embodiments, a L66E substitution in IL-15 has significantly lower (e.g., about 15 fold lower) binding affinity to IL-15Rα and retains little to no IL-15 signaling activity.

It will be appreciated that any one or more of the foregoing IL-15 proteins can be combined with any of the other components described herein, for example, anti-PD-1 Fabs, anti-PD-L1 Fabs, hinge/Fc domains, IL-15Rα proteins, and linkers described herein, to generate one or more activatable proprotein.

IL-15Rα proteins. The activatable proproteins described herein comprise at least one “IL-15Rα protein” (or Interleukin-15 Receptor-α protein), including human IL-15Rα proteins. The IL-15 receptor is composed of three subunits: IL-15Rα, CD122, and CD132. IL-15Rα specifically binds IL-15 with very high affinity, and is capable of binding IL-15 independently of other subunits. Exemplary IL-15Rα protein sequences are provided in Table S2.

TABLE S2
Exemplary IL-15Ra Proteins

| Name | Sequence | SEQ ID NO: |
|---|---|---|
| Human IL-15Rα | MAPRRARGCRTLGLPALLLLLLLRPPATRGITCPPPMSVEHADIWVKSY | 80 |
| FL | SLYSRERYICNSGFKRKAGTSSLTECVLNKATNVAHWTTPSLKCIRDPA | |
| | LVHQRPAPPSTVTTAGVTPQPESLSPSGKEPAASSPSSNNTAATTAAIV | |
| | PGSQLMPSKSPSTGTTEISSHESSHGTPSQTTAKNWELTASASHQPPGV | |
| | YPQGHSDTTVAISTSTVLLCGLSAVSLLACYLKSRQTPPLASVEMEAME | |
| | ALPVTWGTSSRDEDLENCSHHL | |
| Human IL-15Rα | ITCPPPMSVEHADIWVKSYSLYSRERYICNSGFKRKAGTSSLTECVLNK | 81 |
| ECD (31-205) | ATNVAHWTTPSLKCIRDPALVHQRPAPPSTVTTAGVTPQPESLSPSGKE | |
| | PAASSPSSNNTAATTAAIVPGSQLMPSKSPSTGTTEISSHESSHGTPSQ | |
| | TTAKNWELTASASHQPPGVYPQGHSDTT | |
| Human IL-15Rα | ITCPPPMSVEHADIWVKSYSLYSRERYICNSGFKRKAGTSSLTECVLNK | 82 |
| Sushi+ (31- | ATNVAHWTTPSLKCIRDPALVHQRPAPPS | |
| 108) | | |
| Human IL-15Rα | ITCPPPMSVEHADIWVKSYSLYSRERYICNSGFKRKAGTSSLTECVLNK | 83 |
| Sushi (31-95) | ATNVAHWTTPSLKCIR | |
| Human IL-15Rα | ITCPPPMSVEHADIWVKSYSLYSRERYICNSGFKEKAGTSSLTECVLNK | 84 |
| Sushi + with | ATNVAHWTTPSLKCIRDPALVHQRPAPPS | |
| R35E | | |
| Human IL-15Rα | ITCPPPMSVEHADIWVKSYSLYSREEYICNSGFKEKAGTSSLTECVLNK | 85 |
| Sushi + with | ATNVAHWTTPSLKCIRDPALVHQRPAPPS | |
| R26E, R35E | | |
| Human IL-15Rα | ITCPPPMSVEHADIWVKSYSLYSEEEYICNSGFKEKAGTSSLTECVLNK | 86 |
| Sushi + with | ATNVAHWTTPSLKCIRDPALVHQRPAPPS | |
| R24E, R26E, | | |
| R35E | | |
| Human IL-15Rα | IACPPPMSVEHADIWVKSYSLYSRERYICNSGFKRKAGTSSLTECVLNK | 87 |
| Sushi + (31- | ATNVAHWTTPSLKCIRDPALVHQRPAPPS | |
| 108) with T2A | | |
| Human IL-15Rα | IACPPPMSVEHADIWVKSYSLYSRERYICNSGFKRKAGTSSLTECVLNK | 88 |
| Sushi (31-95) | ATNVAHWTTPSLKCIR | |
| with T2A | | |

Thus, in certain embodiments, an IL-15Rα protein comprises, consists, or consists essentially of an amino acid sequence selected from Table S2, or an active variant or fragment thereof that is at least 80, 85, 90, 95, 98, or 100% identical to a sequence selected from Table S2, and which binds to an IL-15 protein. In some embodiments, the IL-15Rα protein comprises, consists, or consists essentially of an amino acid sequence that is at least 80, 85, 90, 95, 98, or 100% to SEQ ID NO: 80 (full-length wild-type human IL-15Rα), or a fragment thereof composed of residues 31-205, 31-108, 31-95 of SEQ ID NO: 80 (full-length wild-type human IL-15Rα), which binds to an IL-15 protein.

Certain IL-15Rα proteins comprise one or more defined amino acid substitutions relative to the exemplary amino acid sequences in Table S2. For instance, certain IL-15α proteins comprise or retain one or more amino acid substitutions at position T2A, R24, R26, and R35 as defined by SEQ ID NO: 82 (IL-15Rα Sushi+), including combinations thereof. Exemplary substitutions include R24E, R26E, and R35E, including combinations thereof. Exemplary combinations include R26E and R35E; and R24E, R26E, and R35E. In specific embodiments, the IL-15α protein comprises SEQ ID NO: 82 or 83 with a T2A substitution, for example, SEQ ID NO: 87 or 88.

It will be appreciated that any one or more of the foregoing IL-15Rα proteins can be combined with any of the other components described herein, for example, anti-PD-1 Fabs, anti-PD-L1 Fabs, hinge/Fc domains, IL-15 proteins, and linkers described herein, to generate one or more activatable proproteins.

Linkers. As noted above, in certain embodiments, each polypeptide comprises at least a first linker and a second linker, typically peptide linkers. In some embodiments, the first linker is a non-cleavable linker, that is, a physiologically-stable linker. In some embodiments, the second linker is a cleavable linker, for example, a cleavable linker that comprises a protease cleavage site. In some instances, the first and second linkers are both cleavable linkers, for example, cleavable linkers that each comprise a protease cleavage site.

In some embodiments, the first linker and/or the second linker are about 1-50 1-40, 1-30, 1-20, 1-10, 1-5, 1-4, 1-3 amino acids in length, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 amino acids in length.

In some embodiments, a cleavable linker comprises at least one protease cleavage site. Suitable protease cleavages sites and self-cleaving peptides are known to the skilled person (see, e.g., Ryan et al., J. Gener. Virol. 78:699-722, 1997; and Scymczak et al., Nature Biotech. 5:589-594, 2004). In some embodiments, the protease cleavage site is cleavable by a protease selected from one or more of a metalloprotease, a serine protease, a cysteine protease, and an aspartic acid protease. In particular embodiments, the protease cleavage site is cleavable by a protease selected from one or more of MMP1, MMP2, MMP3, MMP4, MMP5, MMP6, MMP7, MMP8, MMP9, MMP10, MMP11, MMP12, MMP13, MMP14, TEV protease, matriptase, uPA, FAP, Legumain, PSA, Kallikrein, Cathepsin A, and Cathepsin B.

Exemplary cleavable linker sequences are provided in Table S3.

TABLE S3
Exemplary cleavable linkers

| Name | Sequence | SEQ ID NO: |
|---|---|---|
| PS1 | PLGLSGRSDNQG | 89 |
| PS2 | GGGGSPLGLSGRSDNQGGGGSGGGGS | 90 |
| PS3 | PLGLAGSGRSDNQGA | 91 |
| PS4 | GPLGLAGSGRSDNQGA | 92 |
| PS5 | GGPLGLAGSGRSDNQGA | 93 |
| PS6 | GGSPLGLAGSGRSDNHGGA | 94 |
| PS7 | GGSPLGLAGSGRSDNQGGA | 95 |
| PS8 | GGSPLGLAGSGRSDNRGGA | 96 |
| PS9 | GGSPLGLAGSGRSDNTGGA | 97 |
| PS10 | GGGSGRSDNHGGA | 98 |
| PS11 | GGGGSGRSDNHGGA | 99 |
| PS12 | GGGGSSGRSDNHGGGA | 100 |
| PS13 | GGSGGSSGRSDNHGGGGA | 101 |
| PS14 | GGSGGSSGRSDNHGGSGGA | 102 |
| PS15 | GGSGRSDNHGGA | 103 |
| PS16 | GGSGGSGRSDNHGGA | 104 |
| PS17 | GSPLGLAGS | 105 |
| PS18 | GGSPLGLAGGS | 106 |
| PS19 | GGSPLGLAGGGS | 107 |
| PS20 | GGGSPLGLAGGGS | 108 |
| PS21 | GGGSPLGLAGGGGS | 109 |
| PS22 | GGSGGGPLGLAGGGA | 110 |
| PS23 | SGRSDNA | 111 |
| PS24 | SGRSDND | 112 |
| PS25 | SGRSDNE | 113 |
| PS26 | SGRSDNF | 114 |
| PS27 | SGRSDNG | 115 |
| PS28 | SGRSDNI | 116 |
| PS29 | SGRSDNK | 117 |
| PS30 | SGRSDNL | 118 |
| PS31 | SGRSDNM | 119 |
| PS32 | SGRSDNN | 120 |
| PS33 | SGRSDNP | 121 |
| PS34 | SGRSDNQ | 122 |
| PS35 | SGRSDNR | 123 |
| PS36 | SGRSDNS | 124 |
| PS37 | SGRSDNT | 125 |
| PS38 | SGRSDNV | 126 |
| PS39 | SGRSDNW | 127 |
| PS40 | SGRSDNY | 128 |
| PS41 | PLGLAGSGRSDNR | 129 |
| PS42 | PLGLAGSGRSDNQ | 130 |
| PS43 | PLGLAGSGRSDNT | 131 |
| PS44 | PLGLAGSGRSDNN | 132 |
| PS45 | PLGLAGSGRSDNV | 133 |
| PS46 | PLGLAGSGRSDNW | 134 |
| PS47 | PLGLAGSGRSDNY | 135 |
| | GGGSPLGLAGSGRSDNQGGSGGSGGS | 201 |

Thus, in certain embodiment, a cleavable linker is selected from Table S3. Additional examples of cleavable linkers include an amino acid sequence cleaved by a serine protease such as thrombin, chymotrypsin, trypsin, elastase, kallikrein, or subtilisin. Illustrative examples of thrombin-cleavable amino acid sequences include, but are not limited to: -Gly-Arg-Gly-Asp- (SEQ ID NO: 150), -Gly-Gly-Arg-, -Gly- Arg-Gly-Asp-Asn-Pro- (SEQ ID NO: 151), -Gly-Arg-Gly-Asp-Ser- (SEQ ID NO: 152), -Gly-Arg-Gly-Asp-Ser-Pro-Lys- (SEQ ID NO: 153), -Gly-Pro- Arg-, -Val-Pro-Arg-, and -Phe-Val-Arg-. Illustrative examples of elastase-cleavable amino acid sequences include, but are not limited to: -Ala-Ala-Ala-, -Ala-Ala-Pro-Val- (SEQ ID NO: 154), -Ala-Ala-Pro-Leu- (SEQ ID NO: 155), -Ala-Ala-Pro-Phe-(SEQ ID NO: 156), -Ala-Ala-Pro-Ala- (SEQ ID NO: 157), and -Ala-Tyr-Leu-Val- (SEQ ID NO: 158).

Cleavable linkers also include amino acid sequences that can be cleaved by a matrix metalloproteinase such as collagenase, stromelysin, and gelatinase. Illustrative examples of matrix metalloproteinase-cleavable amino acid sequences include, but are not limited to: -Gly-Pro-Y-Gly-Pro-Z-(SEQ ID NO: 159), -Gly-Pro-, Leu-Gly-Pro-Z-(SEQ ID NO: 160), -Gly-Pro-Ile-Gly-Pro-Z-(SEQ ID NO: 161), and -Ala-Pro-Gly-Leu-Z-(SEQ ID NO: 162), where Y and Z are amino acids. Illustrative examples of collagenase-cleavable amino acid sequences include, but are not limited to: -Pro-Leu-Gly-Pro-D-Arg-Z-(SEQ ID NO: 163), -Pro- Leu-Gly-Leu-Leu-Gly-Z-(SEQ ID NO: 164), -Pro-Gln-Gly-Ile-Ala-Gly-Trp-(SEQ ID NO: 165), -Pro-Leu-Gly-Cys(Me)-His-(SEQ ID NO: 166), -Pro-Leu-Gly-Leu-Tyr-Ala-(SEQ ID NO: 167), -Pro-Leu-Ala-Leu-Trp-Ala-Arg-(SEQ ID NO: 168), and -Pro-Leu-Ala-Tyr-Trp-Ala-Arg-(SEQ ID NO: 169), where Z is an amino acid. An illustrative example of a stromelysin-cleavable amino acid sequence is -Pro-Tyr-Ala-Tyr-Tyr-Met-Arg- (SEQ ID NO: 170); and an example of a gelatinase-cleavable amino acid sequence is -Pro-Leu-Gly-Met-Tyr-Ser-Arg-(SEQ ID NO: 171).

Cleavable linkers also include amino acid sequences that can be cleaved by an angiotensin converting enzyme, such as, for example, -Asp-Lys-Pro-, -Gly-Asp-Lys-Pro-(SEQ ID NO: 172), and -Gly-Ser-Asp-Lys-Pro- (SEQ ID NO: 173). Cleavable linkers also include amino acid sequences that can be degraded by cathepsin B, such as, for example, Val-Cit, Ala-Leu-Ala-Leu-(SEQ ID NO: 174), Gly-Phe-Leu-Gly-(SEQ ID NO: 175), and Phe-Lys.

In particular embodiments, a cleavable linker has a half life at pH 7.4, 25° C., for example, at physiological pH, human body temperature (e.g., in vivo, in serum, in a given tissue), of about or less than about 30 minutes, 1 hour, 2 hours, 3 hours, 4 hours, 5 hours, 6 hours, 12 hours, 18 hours, 24 hours, 36 hours, 48 hours, 72 hours, or 96 hours, or any intervening half-life.

Typically, at least one of the first or second linker is a non-cleavable linker. Exemplary non-cleavable linkers include those disclosed in Maratea et al., Gene 40:39-46, 1985; Murphy et al., PNAS USA. 83:8258-8262, 1986; U.S. Pat. Nos. 4,935,233 and 4,751,180. Particular non-cleavable linker sequences contain Gly, Ser, and/or Asn residues. Other near neutral amino acids, such as Thr and Ala may also be employed in the peptide linker sequence, if desired.

Certain exemplary non-cleavable linkers include Gly, Ser and/or Asn-containing linkers, as follows: [G]ₓ, [S]ₓ, [N]ₓ, [GS]ₓ, [GGS]ₓ, [GSS]ₓ, [GSGS]ₓ (SEQ ID NO: 176), [GGSG]ₓ (SEQ ID NO: 177), [GGGS]ₓ (SEQ ID NO: 178), [GGGGS]ₓ (SEQ ID NO: 179), [GN]ₓ, [GGN]ₓ, [GNN]ₓ, [GNGN]ₓ (SEQ ID NO: 180), [GGNG]ₓ(SEQ ID NO: 181), [GGGN]ₓ (SEQ ID NO: 182), [GGGGN]ₓ(SEQ ID NO: 183) linkers, where x is 1, 2, 3, 4, 5, 6, 7, 8, 9, 10, 11, 12, 13, 14, 15, 16, 17, 18, 19, or 20 or more. Other combinations of these and related amino acids will be apparent to persons skilled in the art.

Additional examples of non-cleavable linkers include the following amino acid sequences:

(SEQ ID NO: 184)
Gly-Gly-Gly-Gly-Ser-Gly-Gly-Gly-Gly-Ser-Gly-Gly-
Gly-Gly-Ser-;
(SEQ ID NO: 185)
Gly-Ser-Gly-Gly-Gly-Gly-Ser-Gly-Gly-Gly-Gly-Ser-
Gly-Gly-Gly-Gly-Ser-Gly-Gly-Gly-Gly-Ser-;
(SEQ ID NO: 186)
Gly-Gly-Gly-Gly-Ser-Gly-Gly-Gly-Gly-Ser-Gly-Gly-
Gly-Gly-Ser-Gly-Gly-Gly-Gly-Ser-Gly-Gly-Gly-Gly-
Ser-Gly-Gly-Gly-Gly-Ser-;
(SEQ ID NO: 187)
Asp-Ala-Ala-Ala-Lys-Glu-Ala-Ala-Ala-Lys-Asp-Ala-
Ala-Ala-Arg-Glu-Ala-Ala-Ala-Arg-Asp-Ala-Ala-Ala-
Lys-;
and
(SEQ ID NO: 188)
Asn-Val-Asp-His-Lys-Pro-Ser-Asn-Thr-Lys-Val-Asp-
Lys-Arg-.

Further non-limiting examples of non-cleavable linkers include DGGGS (SEQ ID NO: 189); TGEKP (SEQ ID NO: 190) (see, e.g., Liu et al., PNAS. 94:5525-5530, 1997); GGRR (SEQ ID NO: 191) (Pomerantz et al. 1995); (GGGGS)ₙ (SEQ ID NO: 192) (Kim et al., PNAS. 93:1156-1160, 1996); EGKSSGSGSESKVD (SEQ ID NO: 193) (Chaudhary et al., PNAS. 87:1066-1070, 1990); KESGSVSSEQLAQFRSLD (SEQ ID NO: 194) (Bird et al., Science. 242:423-426, 1988), GGRRGGGS (SEQ ID NO: 195); LRQRDGERP (SEQ ID NO: 196); LRQKDGGGSERP (SEQ ID NO: 197); LRQKd(GGGS)₂ ERP (SEQ ID NO: 198). In specific embodiments, the linker comprises a Gly3 linker sequence, which includes three glycine residues. In particular embodiments, flexible linkers can be rationally designed using a computer program capable of modeling both DNA-binding sites and the peptides themselves (Desjarlais & Berg, PNAS. 90:2256-2260, 1993; and PNAS. 91:11099-11103, 1994) or by phage display methods.

In some embodiments, the linker comprises a spacer element and a cleavable element so as to make the cleavable element more accessible to the enzyme responsible for cleavage.

It will be appreciated that any one or more of the foregoing linkers can be combined with any one or more of the anti-PD-1 Fabs, anti-PD-L1 Fabs, hinge/Fc domains, IL-15 proteins, and IL-15Rα proteins described herein, to form an activatable proprotein homodimer.

Exemplary activatable proprotein sequences are provided in Table S4.

TABLE S4
Exemplary proIL-15 fusion proteins

| | | |
|---|---|---|
| Name | Sequence | SEQ ID NO: |

P40503699

| | | |
|---|---|---|
| Chains 1 | QVQLVQSGSELKKPGASVKVSCKASGYTFTNYDINWVRQAPGQGLEWMGWIFPG | 136 |
| and 2 | DESTQYNEKFKGRFVFSLDTSVSTAYLQISSLKAEDTAVYYCARQTTATWFAYW | |
| | GQGTLVTVSSASTKGPSVFPLAPSSKSTSGGTAALGCLVKDYFPEPVTVSWNSG | |
| | ALTSGVHTFPAVLQSSGLYSLSSVVTVPSSSLGTQTYICNVNHKPSNTKVDKKV | |
| | EPKSCDKTHTCPPCPAPEAAGGPSVFLFPPKPKDTLMISRTPEVTCVVVDVSHE | |
| | DPEVKFNWYVDGVEVHNAKTKPREEQYNSTYRVVSVLTVLHQDWLNGKEYKCKV | |
| | SNKALAAPIEKTISKAKGQPREPQVYTLPPSRDELTKNQVSLTCLVKGFYPSDI | |
| | AVEWESNGQPENNYKTTPPVLDSDGSFFLYSKLTVDKSRWQQGNVFSCSVMHEA | |
| | LHNHYTQKSLSLSPGPLGLSGRSDNQGNWVNVISDLKKIEDLIQSMHIDATLYT | |
| | ESDVHPSCKVTAMKCFLLELQVISLESGDASIHDTVENLIILANNSLSSNGNVT | |
| | ESGCGECEELEEKNIKEFLQSFVHIVQMFINTAGGGGSPLGLSGRSDNQGGGGS | |
| | GGGGSIACPPPMSVEHADIWVKSYSLYSRERYICNSGFKRKAGTSSLTECVLNK | |
| | ATNVAHWTTPSLKCIRDPALVHQRPAPPS | |
| Chains 3 | DVVMTQSPAFLSVTPGEKVTITCRASQSISDYLHWYQQKPDQAPKLLIKYASQS | 137 |
| and 4 | ISGIPARFSGSGSGTDFTFTISSLEAEDAATYYCQNGHSFPLTFGQGTKLELKR | |
| | TVAAPSVFIFPPSDEQLKSGTASVVCLLNNFYPREAKVQWKVDNALQSGNSQES | |
| | VTEQDSKDSTYSLSSTLTLSKADYEKHKVYACEVTHQGLSSPVTKSENRGEC | |

P40743699

| | | |
|---|---|---|
| Chains 1 | QVQLVQSGSELKKPGASVKVSCKASGYTFTNYDINWVRQAPGQGLEWMGWIFPG | 138 |
| and 2 | DESTQYNEKFKGRFVFSLDTSVSTAYLQISSLKAEDTAVYYCARQTTATWFAYW | |
| | GQGTLVTVSSASTKGPSVFPLAPSSKSTSGGTAALGCLVKDYFPEPVTVSWNSG | |
| | ALTSGVHTFPAVLQSSGLYSLSSVVTVPSSSLGTQTYICNVNHKPSNTKVDKKV | |
| | EPKSCDKTHTCPPCPAPEAAGGPSVFLFPPKPKDTLMISRTPEVTCVVVDVSHE | |
| | DPEVKFNWYVDGVEVHNAKTKPREEQYNSTYRVVSVLTVLHQDWLNGKEYKCKV | |
| | SNKALAAPIEKTISKAKGQPREPQVYTLPPSRDELTKNQVSLTCLVKGFYPSDI | |
| | AVEWESNGQPENNYKTTPPVLDSDGSFFLYSKLTVDKSRWQQGNVFSCSVMHEA | |
| | LHNHYTQKSLSLSPGGGSGGSGGSGGSNWVNVISDLKKIEDLIQSMHIDATLYT | |
| | ESDVHPSCKVTAMKCFLLELQVISLESGDASIHDTVENLIILANNSLSSNGNVT | |
| | ESGCGECEELEEKNIKEFLQSFVHIVQMFINTAGGGGSGGGGSGGSGGSGGGGS | |
| | GGGGSIACPPPMSVEHADIWVKSYSLYSRERYICNSGFKRKAGTSSLTECVLNK | |
| | ATNVAHWTTPSLKCIRDPALVHQRPAPPS | |
| Chains 3 | DVVMTQSPAFLSVTPGEKVTITCRASQSISDYLHWYQQKPDQAPKLLIKYASQS | 139 |
| and 4 | ISGIPARFSGSGSGTDFTFTISSLEAEDAATYYCQNGHSFPLTFGQGTKLELKR | |
| | TVAAPSVFIFPPSDEQLKSGTASVVCLLNNFYPREAKVQWKVDNALQSGNSQES | |
| | VTEQDSKDSTYSLSSTLTLSKADYEKHKVYACEVTHQGLSSPVTKSENRGEC | |

P40751942 (anti-PD-L1 antibody)

| | | |
|---|---|---|
| Chains 1 | QVQLVQSGAEVKKPGSSVKVSCKTSGDTFSTYAISWVRQAPGQGLEWMGGIIPI | 140 |
| and 2 | FGKAHYAQKFQGRVTITADESTSTAYMELSSLRSEDTAVYFCARKFHFVSGSPF | |
| | GMDVWGQGTTVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTK | |
| | VDKKVEPKSCDKTHTCPPCPAPEAAGGPSVFLFPPKPKDTLMISRTPEVTCVVV | |
| | DVSHEDPEVKFNWYVDGVEVHNAKTKPREEQYNSTYRVVSVLTVLHQDWLNGKE | |
| | YKCKVSNKALAAPIEKTISKAKGQPREPQVYTLPPSRDELTKNQVSLTCLVKGF | |
| | YPSDIAVEWESNGQPENNYKTTPPVLDSDGSFFLYSKLTVDKSRWQQGNVFSCS | |
| | VMHEALHNHYTQKSLSLSPGK | |
| Chains 3 | EIVLTQSPATLSLSPGERATLSCRASQSVSSYLAWYQQKPGQAPRLLIYDASNR | 141 |
| and 4 | ATGIPARFSGSGSGTDFTLTISSLEPEDFAVYYCQQRSNWPTFGQGTKVEIKRT | |
| | VAAPSVFIFPPSDEQLKSGTASVVCLLNNFYPREAKVQWKVDNALQSGNSQESV | |
| | TEQDSKDSTYSLSSTLTLSKADYEKHKVYACEVTHQGLSSPVTKSENRGEC | |

P42412037 (anti-PD-1 antibody)

| | | |
|---|---|---|
| Chains 1 | QVQLVESGGGVVQPGRSLRLDCKASGITFSNSGMHWVRQAPGKGLEWVAVIWYD | 142 |
| and 2 | GSKRYYADSVKGRFTISRDNSKNTLFLQMNSLRAEDTAVYYCATNDDYWGQGTL | |
| | VTVSSASTKGPSVFPLAPSSKSTSGGTAALGCLVKDYFPEPVTVSWNSGALTSG | |
| | VHTFPAVLQSSGLYSLSSVVTVPSSSLGTQTYICNVNHKPSNTKVDKKVEPKSC | |
| | DKTHTCPPCPAPEAAGGPSVFLFPPKPKDTLMISRTPEVTCVVVDVSHEDPEVK | |
| | FNWYVDGVEVHNAKTKPREEQYNSTYRVVSVLTVLHQDWLNGKEYKCKVSNKAL | |
| | AAPIEKTISKAKGQPREPQVYTLPPSRDELTKNQVSLTCLVKGFYPSDIAVEWE | |
| | SNGQPENNYKTTPPVLDSDGSFFLYSKLTVDKSRWQQGNVFSCSVMHEALHNHY | |
| | TQKSLSLSPGK | |
| Chains 3 | EIVLTQSPATLSLSPGERATLSCRASQSVSSYLAWYQQKPGQAPRLLIYDASNR | 143 |
| and 4 | ATGIPARFSGSGSGTDFTLTISSLEPEDFAVYYCQQSSNWPRTFGQGTKVEIKR | |
| | TVAAPSVFIFPPSDEQLKSGTASVVCLLNNFYPREAKVQWKVDNALQSGNSQES | |
| | VTEQDSKDSTYSLSSTLTLSKADYEKHKVYACEVTHQGLSSPVTKSENRGEC | |

P53021942

| | | |
|---|---|---|
| Chains 1 | QVQLVQSGAEVKKPGSSVKVSCKTSGDTFSTYAISWVRQAPGQGLEWMGGIIPI | 144 |
| and 2 | FGKAHYAQKFQGRVTITADESTSTAYMELSSLRSEDTAVYFCARKFHFVSGSPF | |
| | GMDVWGQGTTVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTK | |
| | VDKKVEPKSCDKTHTCPPCPAPEAAGGPSVFLFPPKPKDTLMISRTPEVTCVVV | |
| | DVSHEDPEVKFNWYVDGVEVHNAKTKPREEQYNSTYRVVSVLTVLHQDWLNGKE | |
| | YKCKVSNKALAAPIEKTISKAKGQPREPQVYTLPPSRDELTKNQVSLTCLVKGF | |
| | YPSDIAVEWESNGQPENNYKTTPPVLDSDGSFFLYSKLTVDKSRWQQGNVFSCS | |
| | VMHEALHNHYTQKSLSLSPGGGGSGGGSNWVNVISDLKKIEDLIQSMHIDATLY | |
| | TESDVHPSCKVTAMKCFLLELQVISLESGDASIHDTVENLIILANNSLSSNGNV | |
| | TESGCGECEELEEKNIKEFLQSFVHIVQMFINTAGGGGSPLGLSGRSDNQGGGG | |
| | SGGGGSIACPPPMSVEHADIWVKSYSLYSRERYICNSGFKRKAGTSSLTECVLN | |
| | KATNVAHWTTPSLKCIRDPALVHQRPAPPS | |
| Chains 3 | EIVLTQSPATLSLSPGERATLSCRASQSVSSYLAWYQQKPGQAPRLLIYDASNR | 145 |
| and 4 | ATGIPARFSGSGSGTDFTLTISSLEPEDFAVYYCQQRSNWPTFGQGTKVEIKRT | |
| | VAAPSVFIFPPSDEQLKSGTASVVCLLNNFYPREAKVQWKVDNALQSGNSQESV | |
| | TEQDSKDSTYSLSSTLTLSKADYEKHKVYACEVTHQGLSSPVTKSENRGEC | |

P53052037

| | | |
|---|---|---|
| Chains 1 | QVQLVESGGGVVQPGRSLRLDCKASGITFSNSGMHWVRQAPGKGLEWVAVIWYD | 146 |
| and 2 | GSKRYYADSVKGRFTISRDNSKNTLFLQMNSLRAEDTAVYYCATNDDYWGQGTL | |
| | VTVSSASTKGPSVFPLAPSSKSTSGGTAALGCLVKDYFPEPVTVSWNSGALTSG | |
| | VHTFPAVLQSSGLYSLSSVVTVPSSSLGTQTYICNVNHKPSNTKVDKKVEPKSC | |
| | DKTHTCPPCPAPEAAGGPSVFLFPPKPKDTLMISRTPEVTCVVVDVSHEDPEVK | |
| | FNWYVDGVEVHNAKTKPREEQYNSTYRVVSVLTVLHQDWLNGKEYKCKVSNKAL | |
| | AAPIEKTISKAKGQPREPQVYTLPPSRDELTKNQVSLTCLVKGFYPSDIAVEWE | |
| | SNGQPENNYKTTPPVLDSDGSFFLYSKLTVDKSRWQQGNVFSCSVMHEALHNHY | |
| | TQKSLSLSPGGGGSGGGSNWVNVISDLKKIEDLIQSMHIDATLYTESDVHPSCK | |
| | VTAMKCFLLELQVISLESGDASIHDTVENLIILANNSLSSNGNVTESGCGECEE | |
| | LEEKNIKEFLQSFVHIVQMFINTAGGGGSPLGLSGRSDNQGGGGSGGGGSIACP | |
| | PPMSVEHADIWVKSYSLYSRERYICNSGFKRKAGTSSLTECVLNKATNVAHWTT | |
| | PSLKCIRDPALVHQRPAPPS | |
| Chains 3 | EIVLTQSPATLSLSPGERATLSCRASQSVSSYLAWYQQKPGQAPRLLIYDASNR | 147 |
| and 4 | ATGIPARFSGSGSGTDFTLTISSLEPEDFAVYYCQQSSNWPRTFGQGTKVEIKR | |
| | TVAAPSVFIFPPSDEQLKSGTASVVCLLNNFYPREAKVQWKVDNALQSGNSQES | |
| | VTEQDSKDSTYSLSSTLTLSKADYEKHKVYACEVTHQGLSSPVTKSENRGEC | |

P55654367

| | | |
|---|---|---|
| Chains 1 | QVQLVQSGAEVKKPGSSVKVSCKASGFTFTTYYISWVRQAPGQGLEYLGYINMG | 148 |
| and 2 | SGGTNYNEKFKGRVTITADKSTSTAYMELSSLRSEDTAVYYCAIIGYFDYWGQG | |
| | TMVTVSSASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKVDKKVEPK | |
| | SCDKTHTCPPCPAPEAAGGPSVFLFPPKPKDTLMISRTPEVTCVVVDVSHEDPE | |
| | VKFNWYVDGVEVHNAKTKPREEQYNSTYRVVSVLTVLHQDWLNGKEYKCKVSNK | |
| | ALAAPIEKTISKAKGQPREPQVYTLPPSRDELTKNQVSLTCLVKGFYPSDIAVE | |
| | WESNGQPENNYKTTPPVLDSDGSFFLYSKLTVDKSRWQQGNVFSCSVMHEALHN | |
| | HYTQKSLSLSPGGGGSGGGSNWVNVISDLKKIEDLIQSMHIDATLYTESDVHPS | |
| | CKVTAMKCFLLELQVISLESGDASIHDTVENLIILANNSLSSNGNVTESGCGEC | |
| | EELEEKNIKEFLQSFVHIVQMFINTAGGGGSPLGLSGRSDNQGGGGSGGGGSIA | |
| | CPPPMSVEHADIWVKSYSLYSRERYICNSGFKRKAGTSSLTECVLNKATNVAHW | |
| | TTPSLKCIR | |
| Chains 3 | DVVMTQSPLSLPVTLGQPASISCRSSQSLLDSDGGTYLYWFQQRPGQSPRRLIY | 149 |
| and 4 | LVSTLGSGVPDRFSGSGSGTDFTLKISRVEAEDVGVYYCMQLTHWPYTFGQGTK | |
| | LEIKRTVAAPSVFIFPPSDEQLKSGTASVVCLLNNFYPREAKVQWKVDNALQSG | |
| | NSQESVTEQDSKDSTYSLSSTLTLSKADYEKHKVYACEVTHQGLSSPVTKSENR | |
| | GEC | |

P79772037

| | | |
|---|---|---|
| Chains 1 | QVQLVESGGGVVQPGRSLRLDCKASGITFSNSGMHWVRQAPGKGLEWVAVIWYD | 199 |
| and 2 | GSKRYYADSVKGRFTISRDNSKNTLFLQMNSLRAEDTAVYYCATNDDYWGQGTL | |
| | VTVSSASTKGPSVFPLAPSSKSTSGGTAALGCLVKDYFPEPVTVSWNSGALTSG | |
| | VHTFPAVLQSSGLYSLSSVVTVPSSSLGTQTYICNVNHKPSNTKVDKKVEPKSC | |
| | DKTHTCPPCPAPEAAGGPSVFLFPPKPKDTLMISRTPEVTCVVVDVSHEDPEVK | |
| | FNWYVDGVEVHNAKTKPREEQYNSTYRVVSVLTVLHQDWLNGKEYKCKVSNKAL | |
| | AAPIEKTISKAKGQPREPQVYTLPPSRDELTKNQVSLTCLVKGFYPSDIAVEWE | |
| | SNGQPENNYKTTPPVLDSDGSFFLYSKLTVDKSRWQQGNVFSCSVMHEALHNHY | |
| | TQKSLSLSPGGGGSGGGSNWVNVISDLKKIEDLIQSMHIDATLYTESDVHPSCK | |
| | VTAMKCFLLELQVISLESGDASIHDTVENLIILANNSLSSNGNVTESGCGECEE | |
| | LEEKNIKEFLQSFVHIVQMFINTAGGGSPLGLAGSGRSDNQGGSGGSGGSIACP | |
| | PPMSVEHADIWVKSYSLYSRERYICNSGFKRKAGTSSLTECVLNKATNVAHWTT | |
| | PSLKCIRDPALVHQRPAPPS | |
| Chains 3 | EIVLTQSPATLSLSPGERATLSCRASQSVSSYLAWYQQKPGQAPRLLIYDASNR | 200 |
| and 4 | ATGIPARFSGSGSGTDFTLTISSLEPEDFAVYYCQQSSNWPRTFGQGTKVEIKR | |
| | TVAAPSVFIFPPSDEQLKSGTASVVCLLNNFYPREAKVQWKVDNALQSGNSQES | |
| | VTEQDSKDSTYSLSSTLTLSKADYEKHKVYACEVTHQGLSSPVTKSENRGEC | |

Thus, in certain embodiments, an activatable proprotein comprises a first and second polypeptide that comprises, consists, or consists essentially of an amino acid sequence that is at least 80, 85, 90, 95, 98, or 100% identical to a sequence selected from Table S4 (i.e., chains 1 and 2), and a VL/CL region polypeptide that is at least 80, 85, 90, 95, 98, or 100% identical to the corresponding sequence from Table S4 (i.e., chains 3 and 4).

### Methods of Use and Pharmaceutical Compositions

Certain embodiments include methods of treating, ameliorating the symptoms of, and/or reducing the progression of, a disease or condition in a subject in need thereof, comprising administering to the subject at least one activatable proprotein, as described herein. Also included are methods of enhancing an immune response in a subject comprising administering to the subject at least one activatable proprotein, as described herein. In particular embodiments, the disease is a cancer. In some embodiments, the cancer expresses or over-expresses PD-L1 or B7H3.

In some embodiments, following administration, the activatable proprotein is activated through protease cleavage in a cell or tissue, which exposes the binding site of the IL-15 protein that binds to the IL-15β/γc chain present on the surface of the immune cell in vitro or in vivo, and thereby generates an activated protein. In particular embodiments, the protease cleavage occurs in a cancer cell or cancer tissue. Typically, the activated protein has at least one immune-stimulating IL-15 activity, for example, by binding to the IL-15β/γc chain present on the surface of an immune cell in vivo, and thereby stimulating the immune cell. In particular embodiments, the immune cell is selected from one or more of a T cell, a B cell, a natural killer cell, a monocyte, and a macrophage.

In some embodiments, administration and activation of the activatable proprotein, to generate an activated protein, increases an anti-cancer immune response in the subject by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control. In some embodiments, administration and activation of the activatable proprotein, to generate an activated protein, increases cancer cell-killing in the subject by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control.

In certain embodiments, the activated anti-PD-1 Fab/IL-15 protein stimulates an increased (e.g., synergistically increased) anti-cancer immune response relative to either the corresponding anti-PD-1 Fab (or corresponding anti-PD-1 antibody) alone and/or the corresponding IL-15 protein alone, for example, a 2-fold, 2.5-fold, 3-fold, 3.5-fold, 4-fold, 4.5-fold, 5-fold, 6-fold, 7-fold, 8-fold, 9-fold, or 10-fold or more increase in the anti-cancer immune response relative to each component alone. In certain embodiments, the activated anti-PD-1 Fab/IL-15 protein stimulates increased (e.g., synergistically increased) cancer cell-killing activity relative to either the corresponding anti-PD-1 Fab (or corresponding anti-PD-1 antibody) alone and/or the corresponding IL-15 protein alone, for example, a 2-fold, 2.5-fold, 3-fold, 3.5-fold, 4-fold, 4.5-fold, 5-fold, 6-fold, 7-fold, 8-fold, 9-fold, or 10-fold or more increase in the cancer cell-killing activity relative to each component alone.

In certain embodiments, the activated anti-PD-L1 Fab/IL-15 protein stimulates an increased (e.g., synergistically increased) anti-cancer immune response relative to either the corresponding anti-PD-L1 Fab (or corresponding anti-PD-L1 antibody) alone and/or the corresponding IL-15 protein alone, for example, a 2-fold, 2.5-fold, 3-fold, 3.5-fold, 4-fold, 4.5-fold, 5-fold, 6-fold, 7-fold, 8-fold, 9-fold, or 10-fold or more increase in the anti-cancer immune response relative to each component alone. In certain embodiments, the activated anti-PD-L1 Fab/IL-15 protein stimulates increased (e.g., synergistically increased) cancer cell-killing activity relative to either the corresponding anti-PD-L1 Fab (or corresponding anti-PD-L1 antibody) alone and/or the corresponding IL-15 protein alone, for example, a 2-fold, 2.5-fold, 3-fold, 3.5-fold, 4-fold, 4.5-fold, 5-fold, 6-fold, 7-fold, 8-fold, 9-fold, or 10-fold or more increase in the cancer cell-killing activity relative to each component alone.

In certain embodiments, the activated anti-B7H3 Fab/IL-15 protein stimulates an increased (e.g., synergistically increased) anti-cancer immune response relative to either the corresponding anti-B7H3 Fab (or corresponding anti-B7H3 antibody) alone and/or the corresponding IL-15 protein alone, for example, a 2-fold, 2.5-fold, 3-fold, 3.5-fold, 4-fold, 4.5-fold, 5-fold, 6-fold, 7-fold, 8-fold, 9-fold, or 10-fold or more increase in the anti-cancer immune response relative to each component alone. In certain embodiments, the activated anti-B7H3 Fab/IL-15 protein stimulates increased (e.g., synergistically increased) cancer cell-killing activity relative to either the corresponding anti-B7H3 Fab (or corresponding anti-B7H3 antibody) alone and/or the corresponding IL-15 protein alone, for example, a 2-fold, 2.5-fold, 3-fold, 3.5-fold, 4-fold, 4.5-fold, 5-fold, 6-fold, 7-fold, 8-fold, 9-fold, or 10-fold or more increase in the cancer cell-killing activity relative to each component alone.

In some embodiments, the disease is a cancer, that is, the subject in need thereof has or is suspected of having a cancer. Certain embodiments thus include methods of treating, ameliorating the symptoms of, or inhibiting the progression of, a cancer in a subject in need thereof, comprising administering to the subject at least one activatable proprotein, as described herein. In particular embodiments, the cancer is a primary cancer or a metastatic cancer. In specific embodiments, the cancer is selected from one or more of melanoma (optionally metastatic melanoma), kidney cancer (optionally renal cell carcinoma), pancreatic cancer, bone cancer, prostate cancer, small cell lung cancer, non-small cell lung cancer (NSCLC), mesothelioma, leukemia (optionally lymphocytic leukemia, chronic myelogenous leukemia, acute myeloid leukemia, or relapsed acute myeloid leukemia), multiple myeloma, lymphoma, hepatoma (hepatocellular carcinoma), sarcoma, B-cell malignancy, breast cancer, ovarian cancer, colorectal cancer, glioma, glioblastoma multiforme, meningioma, pituitary adenoma, vestibular schwannoma, primary CNS lymphoma, primitive neuroectodermal tumor (medulloblastoma), bladder cancer, uterine cancer, esophageal cancer, brain cancer, head and neck cancers, cervical cancer, testicular cancer, thyroid cancer, and stomach cancer

In some embodiments, as noted above, the cancer is a metastatic cancer. Further to the above cancers, exemplary metastatic cancers include, without limitation, bladder cancers which have metastasized to the bone, liver, and/or lungs; breast cancers which have metastasized to the bone, brain, liver, and/or lungs; colorectal cancers which have metastasized to the liver, lungs, and/or peritoneum; kidney cancers which have metastasized to the adrenal glands, bone, brain, liver, and/or lungs; lung cancers which have metastasized to the adrenal glands, bone, brain, liver, and/or other lung sites; melanomas which have metastasized to the bone, brain, liver, lung, and/or skin/muscle; ovarian cancers which have metastasized to the liver, lung, and/or peritoneum; pancreatic cancers which have metastasized to the liver, lung, and/or peritoneum; prostate cancers which have metastasized to the adrenal glands, bone, liver, and/or lungs; stomach cancers which have metastasized to the liver, lung, and/or peritoneum; thyroid cancers which have metastasized to the bone, liver, and/or lungs; and uterine cancers which have metastasized to the bone, liver, lung, peritoneum, and/or vagina; among others.

In certain embodiments, as noted herein, the cancer expresses or over-expresses PD-L1 or B7H3. PD-L1 and B7H3 expression levels in a sample of tissue (e.g., cancer tissue) can be determined by any variety of methods. For example, PD-L1 or B7H3 protein levels can be determined by immunohistochemistry (IHC) including chromogenic or fluorescent IHC, enzyme linked immunosorbent assay (ELISA), or Western blot on a human protein or gene, among other assays. PD-L1 or B7H3 mRNA levels can be measured, for example, by RT-PCR, for example, quantitative competitive (QC) RT-PCR, among other techniques known in the art. Certain embodiments thus include the step of determining or detecting or measuring PD-L1 and/or B7H3 levels in a tissue sample from a subject in need thereof. Also included is the step of comparing the PD-L1 levels in a tissue sample relative to that of a control or reference. Certain embodiments include the step of determining PD-L1 and/or B7H3 levels in a sample of cancer tissue from the subject (e.g., biopsy tissue), and administering the activatable proprotein homodimer if the cancer tissue from the subject expresses or over-expresses PD-L1 or B7H3.

The methods for treating cancers can be combined with other therapeutic modalities. For example, a combination therapy described herein can be administered to a subject before, during, or after other therapeutic interventions, including symptomatic care, radiotherapy, surgery, transplantation, hormone therapy, photodynamic therapy, antibiotic therapy, or any combination thereof. Symptomatic care includes administration of corticosteroids, to reduce cerebral edema, headaches, cognitive dysfunction, and emesis, and administration of anti-convulsants, to reduce seizures. Radiotherapy includes whole-brain irradiation, fractionated radiotherapy, and radiosurgery, such as stereotactic radiosurgery, which can be further combined with traditional surgery.

Certain embodiments thus include combination therapies for treating cancers, including methods of treating ameliorating the symptoms of, or inhibiting the progression of, a cancer in a subject in need thereof, comprising administering to the subject at least one activatable proprotein described herein in combination with at least one additional agent, for example, a chemotherapeutic agent, a hormonal therapeutic agent, and/or a kinase inhibitor. In some embodiments, administering the at least one activatable proprotein enhances the susceptibility of the cancer to the additional agent (for example, chemotherapeutic agent, hormonal therapeutic agent, and or kinase inhibitor) by about or at least about 5, 10, 15, 20, 25, 30, 35, 40, 45, 50, 60, 70, 80, 90, 100, 200, 300, 400, 500, 600, 700, 800, 900, 1000, 2000% or more relative to the additional agent alone.

Certain combination therapies employ one or more chemotherapeutic agents, for example, small molecule chemotherapeutic agents. Non-limiting examples of chemotherapeutic agents include alkylating agents, anti-metabolites, cytotoxic antibiotics, topoisomerase inhibitors (type 1 or type II), an anti-microtubule agents, among others.

Examples of alkylating agents include nitrogen mustards (e.g., mechlorethamine, cyclophosphamide, mustine, melphalan, chlorambucil, ifosfamide, and busulfan), nitrosoureas (e.g., N-Nitroso-N-methylurea (MNU), carmustine (BCNU), lomustine (CCNU), semustine (MeCCNU), fotemustine, and streptozotocin), tetrazines (e.g., dacarbazine, mitozolomide, and temozolomide), aziridines (e.g., thiotepa, mytomycin, and diaziquone (AZQ)), cisplatins and derivatives thereof (e.g., carboplatin and oxaliplatin), and non-classical alkylating agents (optionally procarbazine and hexamethylmelamine).

Examples of anti-metabolites include anti-folates (e.g., methotrexate and pemetrexed), fluoropyrimidines (e.g., 5-fluorouracil and capecitabine), deoxynucleoside analogues (e.g., ancitabine, enocitabine, cytarabine, gemcitabine, decitabine, azacitidine, fludarabine, nelarabine, cladribine, clofarabine, fludarabine, and pentostatin), and thiopurines (e.g., thioguanine and mercaptopurine);

Examples of cytotoxic antibiotics include anthracyclines (e.g., doxorubicin, daunorubicin, epirubicin, idarubicin, pirarubicin, aclarubicin, and mitoxantrone), bleomycins, mitomycin C, mitoxantrone, and actinomycin. Examples of topoisomerase inhibitors include camptothecin, irinotecan, topotecan, etoposide, doxorubicin, mitoxantrone, teniposide, novobiocin, merbarone, and aclarubicin.

Examples of anti-microtubule agents include taxanes (e.g., paclitaxel and docetaxel) and vinca alkaloids (e.g., vinblastine, vincristine, vindesine, vinorelbine).

The skilled artisan will appreciate that the various chemotherapeutic agents described herein can be combined with any one or more of the activatable proproteins described herein, and used according to any one or more of the methods or compositions described herein.

Certain combination therapies employ at least one hormonal therapeutic agent. General examples of hormonal therapeutic agents include hormonal agonists and hormonal antagonists. Particular examples of hormonal agonists include progestogen (progestin), corticosteroids (e.g., prednisolone, methylprednisolone, dexamethasone), insulin like growth factors, VEGF derived angiogenic and lymphangiogenic factors (e.g., VEGF-A, VEGF-A145, VEGF-A165, VEGF-C, VEGF-D, PIGF-2), fibroblast growth factor (FGF), galectin, hepatocyte growth factor (HGF), platelet derived growth factor (PDGF), transforming growth factor (TGF)-beta, androgens, estrogens, and somatostatin analogs. Examples of hormonal antagonists include hormone synthesis inhibitors such as aromatase inhibitors and gonadotropin-releasing hormone (GnRH)s agonists (e.g., leuprolide, goserelin, triptorelin, histrelin) including analogs thereof. Also included are hormone receptor antagonist such as selective estrogen receptor modulators (SERMs; e.g., tamoxifen, raloxifene, toremifene) and anti-androgens (e.g., flutamide, bicalutamide, nilutamide).

Also included are hormonal pathway inhibitors such as antibodies directed against hormonal receptors. Examples include inhibitors of the the IGF receptor (e.g., IGF-IR1) such as cixutumumab, dalotuzumab, figitumumab, ganitumab, istiratumab, and robatumumab; inhibitors of the vascular endothelial growth factor receptors 1, 2 or 3 (VEGFR1, VEGFR2 or VEGFR3) such as alacizumab pegol, bevacizumab, icrucumab, ramucirumab; inhibitors of the TGF-beta receptors R1, R2, and R3 such as fresolimumab and metelimumab; inhibitors of c-Met such as naxitamab; inhibitors of the EGF receptor such as cetuximab, depatuxizumab mafodotin, futuximab, imgatuzumab, laprituximab emtansine, matuzumab, modotuximab, necitumumab, nimotuzumab, panitumumab, tomuzotuximab, and zalutumumab; inhibitors of the FGF receptor such as aprutumab ixadotin and bemarituzumab; and inhibitors of the PDGF receptor such as olaratumab and tovetumab.

The skilled artisan will appreciate that the various hormonal therapeutic agents described herein can be combined with any one or more of the various activatable proproteins described herein, and used according to any one or more of the methods or compositions described herein.

Certain combination therapies employ at least one kinase inhibitor, including tyrosine kinase inhibitors. Examples of kinase inhibitors include, without limitation, adavosertib, afanitib, aflibercept, axitinib, bevacizumab, bosutinib, cabozantinib, cetuximab, cobimetinib, crizotinib, dasatinib, entrectinib, erdafitinib, erlotinib, fostamitinib, gefitinib, ibrutinib, imatinib, lapatinib, lenvatinib, mubritinib, nilotinib, panitumumab, pazopanib, pegaptanib, ponatinib, ranibizumab, regorafenib, ruxolitinib, sorafenib, sunitinib, SU6656, tofacitinib, trastuzumab, vandetanib, and vemuafenib.

The skilled artisan will appreciate that the various kinase inhibitors described herein can be combined with any one or more of the various activatable proproteins described herein, and used according to any one or more of the methods or compositions described herein.

In some embodiments, the methods and pharmaceutical compositions described herein increase median survival time of a subject by 4 weeks, 5 weeks, 6 weeks, 7 weeks, 8 weeks, 9 weeks, 10 weeks, 15 weeks, 20 weeks, 25 weeks, 30 weeks, 40 weeks, or longer. In certain embodiments, the methods and pharmaceutical compositions described herein increase median survival time of a subject by 1 year, 2 years, 3 years, or longer. In some embodiments, the methods and pharmaceutical compositions increase progression-free survival by 2 weeks, 3 weeks, 4 weeks, 5 weeks, 6 weeks, 7 weeks, 8 weeks, 9 weeks, 10 weeks or longer. In certain embodiments, the methods and pharmaceutical compositions described herein increase progression-free survival by 1 year, 2 years, 3 years, or longer.

In certain embodiments, the methods and therapeutic compositions described herein are sufficient to result in tumor regression, as indicated by a statistically significant decrease in the amount of viable tumor, for example, at least a 10%, 20%, 30%, 40%, 50% or greater decrease in tumor mass, or by altered (e.g., decreased with statistical significance) scan dimensions. In certain embodiments, the methods and therapeutic compositions described herein are sufficient to result in stable disease.

In certain embodiments, the methods and therapeutic compositions described herein are sufficient to result in clinically relevant reduction in symptoms of a particular disease indication known to the skilled clinician.

For in vivo use, as noted above, for the treatment of human or non-human mammalian disease or testing, the agents described herein are generally incorporated into one or more therapeutic or pharmaceutical compositions prior to administration, including veterinary therapeutic compositions.

Thus, certain embodiments relate to pharmaceutical or therapeutic compositions that comprise at least one activatable proprotein, as described herein. In some instances, a pharmaceutical or therapeutic composition comprises one or more of the activatable proproteins described herein in combination with a pharmaceutically- or physiologically-acceptable carrier or excipient. Certain pharmaceutical or therapeutic compositions further comprise at least one additional agent, for example, a chemotherapeutic agent, a hormonal therapeutic agent, and/or a kinase inhibitor as described herein.

Some therapeutic compositions comprise (and certain methods utilize) only one activatable proprotein. Certain therapeutic compositions comprise (and certain methods utilize) a mixture of at least two, three, four, or five different activatable proproteins.

In particular embodiments, the pharmaceutical or therapeutic compositions comprising at least one activatable proprotein is substantially pure on a protein basis or a weight-weight basis, for example, the composition has a purity of at least about 80%, 85%, 90%, 95%, 98%, or 99% on a protein basis or a weight-weight basis.

In some embodiments, the activatable proproteins described herein do not form aggregates, have a desired solubility, and/or have an immunogenicity profile that is suitable for use in humans, as known in the art. Thus, in some embodiments, the therapeutic composition comprising an activatable proprotein is substantially aggregate-free. For example, certain compositions comprise less than about 10% (on a protein basis) high molecular weight aggregated proteins, or less than about 5% high molecular weight aggregated proteins, or less than about 4% high molecular weight aggregated proteins, or less than about 3% high molecular weight aggregated proteins, or less than about 2% high molecular weight aggregated proteins, or less than about 1% high molecular weight aggregated proteins. Some compositions comprise an activatable proprotein that is at least about 50%, about 60%, about 70%, about 80%, about 90% or about 95% monodisperse with respect to its apparent molecular mass.

In some embodiments, the activatable proprotein are concentrated to about or at least about 0.1 mg/ml, 0.2 mg/ml, 0.3 mg/ml, 0.4 mg/ml, 0.5 mg/ml, 0.6, 0.7, 0.8, 0.9, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11, 12, 13, 14 or 15 mg/ml and are formulated for biotherapeutic uses.

To prepare a therapeutic or pharmaceutical composition, an effective or desired amount of one or more agents is mixed with any pharmaceutical carrier(s) or excipient known to those skilled in the art to be suitable for the particular agent and/or mode of administration. A pharmaceutical carrier may be liquid, semi-liquid or solid. Solutions or suspensions used for parenteral, intradermal, subcutaneous or topical application may include, for example, a sterile diluent (such as water), saline solution (e.g., phosphate buffered saline; PBS), fixed oil, polyethylene glycol, glycerin, propylene glycol or other synthetic solvent; antimicrobial agents (such as benzyl alcohol and methyl parabens); antioxidants (such as ascorbic acid and sodium bisulfite) and chelating agents (such as ethylenediaminetetraacetic acid (EDTA)); buffers (such as acetates, citrates and phosphates). If administered intravenously (e.g., by IV infusion), suitable carriers include physiological saline or phosphate buffered saline (PBS), and solutions containing thickening and solubilizing agents, such as glucose, polyethylene glycol, polypropylene glycol and mixtures thereof.

Administration of agents described herein, in pure form or in an appropriate therapeutic or pharmaceutical composition, can be carried out via any of the accepted modes of administration of agents for serving similar utilities. The therapeutic or pharmaceutical compositions can be prepared by combining an agent-containing composition with an appropriate physiologically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. In addition, other pharmaceutically active ingredients (including other small molecules as described elsewhere herein) and/or suitable excipients such as salts, buffers and stabilizers may, but need not, be present within the composition.

Administration may be achieved by a variety of different routes, including oral, parenteral, nasal, intravenous, intradermal, intramuscular, subcutaneous or topical. Preferred modes of administration depend upon the nature of the condition to be treated or prevented. Particular embodiments include administration by IV infusion.

Carriers can include, for example, pharmaceutically- or physiologically-acceptable carriers, excipients, or stabilizers that are non-toxic to the cell or mammal being exposed thereto at the dosages and concentrations employed. Often the physiologically-acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as polysorbate 20 (TWEEN™) polyethylene glycol (PEG), and poloxamers (PLURONICS™), and the like.

In some embodiments, one or more agents can be entrapped in microcapsules prepared, for example, by coacervation techniques or by interfacial polymerization (for example, hydroxymethylcellulose or gelatin-microcapsules and poly-(methylmethacylate)microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980). The particle(s) or liposomes may further comprise other therapeutic or diagnostic agents.

The precise dosage and duration of treatment is a function of the disease being treated and may be determined empirically using known testing protocols or by testing the compositions in model systems known in the art and extrapolating therefrom. Controlled clinical trials may also be performed. Dosages may also vary with the severity of the condition to be alleviated. A pharmaceutical composition is generally formulated and administered to exert a therapeutically useful effect while minimizing undesirable side effects. The composition may be administered one time, or may be divided into a number of smaller doses to be administered at intervals of time. For any particular subject, specific dosage regimens may be adjusted over time according to the individual need.

Typical routes of administering these and related therapeutic or pharmaceutical compositions thus include, without limitation, oral, topical, transdermal, inhalation, parenteral, sublingual, buccal, rectal, vaginal, and intranasal. The term parenteral as used herein includes subcutaneous injections, intravenous, intramuscular, intrasternal injection or infusion techniques. Therapeutic or pharmaceutical compositions according to certain embodiments of the present disclosure are formulated so as to allow the active ingredients contained therein to be bioavailable upon administration of the composition to a subject or patient. Compositions that will be administered to a subject or patient may take the form of one or more dosage units, where for example, a tablet may be a single dosage unit, and a container of a herein described agent in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will typically contain a therapeutically effective amount of an agent described herein, for treatment of a disease or condition of interest.

A therapeutic or pharmaceutical composition may be in the form of a solid or liquid. In one embodiment, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi-solid, semi-liquid, suspension and gel forms are included within the forms considered herein as either solid or liquid. Certain embodiments include sterile, injectable solutions.

As a solid composition for oral administration, the pharmaceutical composition may be formulated into a powder, granule, compressed tablet, pill, capsule, chewing gum, wafer or the like. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the pharmaceutical composition is in the form of a capsule, for example, a gelatin capsule, it may contain, in addition to materials of the above type, a liquid carrier such as polyethylene glycol or oil.

The therapeutic or pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid therapeutic or pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid therapeutic or pharmaceutical composition intended for either parenteral or oral administration should contain an amount of an agent such that a suitable dosage will be obtained. Typically, this amount is at least 0.01% of the agent of interest in the composition. When intended for oral administration, this amount may be varied to be between 0.1 and about 70% of the weight of the composition. Certain oral therapeutic or pharmaceutical compositions contain between about 4% and about 75% of the agent of interest. In certain embodiments, therapeutic or pharmaceutical compositions and preparations are prepared so that a parenteral dosage unit contains between 0.01 to 10% by weight of the agent of interest prior to dilution.

The therapeutic or pharmaceutical compositions may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a therapeutic or pharmaceutical composition for topical administration. If intended for transdermal administration, the composition may include a transdermal patch or iontophoresis device.

The therapeutic or pharmaceutical compositions may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter, and polyethylene glycol.

The therapeutic or pharmaceutical composition may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The therapeutic or pharmaceutical compositions in solid or liquid form may include a component that binds to agent and thereby assists in the delivery of the compound. Suitable components that may act in this capacity include monoclonal or polyclonal antibodies, one or more proteins or a liposome.

The therapeutic or pharmaceutical composition may consist essentially of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols may be delivered in single phase, bi-phasic, or tri-phasic systems in order to deliver the active ingredient(s). Delivery of the aerosol includes the necessary container, activators, valves, subcontainers, and the like, which together may form a kit. One of ordinary skill in the art, without undue experimentation may determine preferred aerosols.

The compositions described herein may be prepared with carriers that protect the agents against rapid elimination from the body, such as time release formulations or coatings. Such carriers include controlled release formulations, such as, but not limited to, implants and microencapsulated delivery systems, and biodegradable, biocompatible polymers, such as ethylene vinyl acetate, polyanhydrides, polyglycolic acid, polyorthoesters, polylactic acid and others known to those of ordinary skill in the art.

The therapeutic or pharmaceutical compositions may be prepared by methodology well known in the pharmaceutical art. For example, a therapeutic or pharmaceutical composition intended to be administered by injection may comprise one or more of salts, buffers and/or stabilizers, with sterile, distilled water so as to form a solution. A surfactant may be added to facilitate the formation of a homogeneous solution or suspension. Surfactants are compounds that non-covalently interact with the agent so as to facilitate dissolution or homogeneous suspension of the agent in the aqueous delivery system.

The therapeutic or pharmaceutical compositions may be administered in a therapeutically effective amount, which will vary depending upon a variety of factors including the activity of the specific compound employed; the metabolic stability and length of action of the compound; the age, body weight, general health, sex, and diet of the subject; the mode and time of administration; the rate of excretion; the drug combination; the severity of the particular disorder or condition; and the subject undergoing therapy. In some instances, a therapeutically effective daily dose is (for a 70 kg mammal) from about 0.001 mg/kg (i.e., ˜0.07 mg) to about 100 mg/kg (i.e., ˜7.0 g); preferably a therapeutically effective dose is (for a 70 kg mammal) from about 0.01 mg/kg (i.e., ˜0.7 mg) to about 50 mg/kg (i.e., ˜3.5 g); more preferably a therapeutically effective dose is (for a 70 kg mammal) from about 1 mg/kg (i.e., ˜70 mg) to about 25 mg/kg (i.e., ˜1.75 g). In some embodiments, the therapeutically effective dose is administered on a weekly, bi-weekly, or monthly basis. In specific embodiments, the therapeutically effective dose is administered on a weekly, bi-weekly, or monthly basis, for example, at a dose of about 1-10 or 1-5 mg/kg, or about 1, 2, 3, 4, 5, 6, 7, 8, 9, or 10 mg/kg.

The combination therapies described herein may include administration of a single pharmaceutical dosage formulation, which contains an activatable proprotein and an additional therapeutic agent (e.g., chemotherapeutic agent, hormonal therapeutic agent, kinase inhibitor), as well as administration of compositions comprising an activatable proprotein and an additional therapeutic agent in its own separate pharmaceutical dosage formulation. For example, an activatable proprotein and additional therapeutic agent can be administered to the subject together in a single oral dosage composition such as a tablet or capsule, or each agent administered in separate oral dosage formulations. Similarly, an activatable proprotein and additional therapeutic agent can be administered to the subject together in a single parenteral dosage composition such as in a saline solution or other physiologically acceptable solution, or each agent administered in separate parenteral dosage formulations. As another example, for cell-based therapies, an activatable proprotein can be mixed with the cells prior to administration, administered as part of a separate composition, or both. Where separate dosage formulations are used, the compositions can be administered at essentially the same time, i.e., concurrently, or at separately staggered times, i.e., sequentially and in any order; combination therapy is understood to include all these regimens.

Also included are patient care kits, comprising (a) at least one activatable proprotein, as described herein; and optionally (b) at least one additional therapeutic agent (e.g., chemotherapeutic agent, hormonal therapeutic agent, kinase inhibitor). In certain kits, (a) and (b) are in separate therapeutic compositions. In some kits, (a) and (b) are in the same therapeutic composition.

The kits herein may also include a one or more additional therapeutic agents or other components suitable or desired for the indication being treated, or for the desired diagnostic application. The kits herein can also include one or more syringes or other components necessary or desired to facilitate an intended mode of delivery (e.g., stents, implantable depots, etc.).

In some embodiments, a patient care kit contains separate containers, dividers, or compartments for the composition(s) and informational material(s). For example, the composition(s) can be contained in a bottle, vial, or syringe, and the informational material(s) can be contained in association with the container. In some embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of an activatable proprotein and optionally at least one additional therapeutic agent. For example, the kit includes a plurality of syringes, ampules, foil packets, or blister packs, each containing a single unit dose of an activatable proprotein and optionally at least one additional therapeutic agent. The containers of the kits can be air tight, waterproof (e.g., impermeable to changes in moisture or evaporation), and/or light-tight.

The patient care kit optionally includes a device suitable for administration of the composition, e.g., a syringe, inhalant, dropper (e.g., eye dropper), swab (e.g., a cotton swab or wooden swab), or any such delivery device. In some embodiments, the device is an implantable device that dispenses metered doses of the agent(s). Also included are methods of providing a kit, e.g., by combining the components described herein.

### Expression and Purification Systems

Certain embodiments include methods and related compositions for expressing and purifying an activatable proprotein described herein. Such recombinant activatable proproteins can be conveniently prepared using standard protocols as described for example in Sambrook, et al., (1989, supra), in particular Sections 16 and 17; Ausubel et al., (1994, supra), in particular Chapters 10 and 16; and Coligan et al., Current Protocols in Protein Science (John Wiley & Sons, Inc. 1995-1997), in particular Chapters 1, 5 and 6. As one general example, activatable proproteins may be prepared by a procedure including one or more of the steps of: (a) preparing one or more vectors or constructs comprising one or more polynucleotide sequences that encode a first and second polypeptide described herein, and a VL/CL region of an anti-PD-1 or anti-PD-L1 or anti-B7H3 Fab region described herein, which are operably linked to one or more regulatory elements; (b) introducing the one or more vectors or constructs into one or more host cells; (c) culturing the one or more host cell to express the first and second polypeptides and the VL/CL regions, which bind together to form an activatable proprotein; and (d) isolating the activatable proprotein from the host cell.

To express a desired polypeptide, a nucleotide sequence encoding a first and/or second polypeptide chain of an activatable proprotein may be inserted into appropriate expression vector(s), i.e., vector(s) which contain the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described in Sambrook et al., Molecular Cloning, A Laboratory Manual (1989), and Ausubel et al., Current Protocols in Molecular Biology (1989).

A variety of expression vector/host systems are known and may be utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems, including mammalian cell and more specifically human cell systems.

The “control elements” or “regulatory sequences” present in an expression vector are those non-translated regions of the vector-enhancers, promoters, 5′ and 3′ untranslated regions-which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the PBLUESCRIPT phagemid (Stratagene, La Jolla, Calif.) or PSPORT1 plasmid (Gibco BRL, Gaithersburg, Md.) and the like may be used. In mammalian cell systems, promoters from mammalian genes or from mammalian viruses are generally preferred. If it is necessary to generate a cell line that contains multiple copies of the sequence encoding a polypeptide, vectors based on SV40 or EBV may be advantageously used with an appropriate selectable marker.

In bacterial systems, a number of expression vectors may be selected depending upon the use intended for the expressed polypeptide. For example, when large quantities are needed, vectors which direct high level expression of fusion proteins that are readily purified may be used. Such vectors include, but are not limited to, the multifunctional E. coli cloning and expression vectors such as BLUESCRIPT (Stratagene), in which the sequence encoding the polypeptide of interest may be ligated into the vector in frame with sequences for the amino-terminal Met and the subsequent 7 residues of β-galactosidase so that a hybrid protein is produced; pIN vectors (Van Heeke & Schuster, J. Biol. Chem. 264:5503 5509 (1989)); and the like. pGEX Vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

Certain embodiments employ E. coli-based expression systems (see, e.g., Structural Genomics Consortium et al., Nature Methods. 5:135-146, 2008). These and related embodiments may rely partially or totally on ligation-independent cloning (LIC) to produce a suitable expression vector. In specific embodiments, protein expression may be controlled by a T7 RNA polymerase (e.g., pET vector series). These and related embodiments may utilize the expression host strain BL21(DE3), a λDE3 lysogen of BL21 that supports T7-mediated expression and is deficient in lon and ompT proteases for improved target protein stability. Also included are expression host strains carrying plasmids encoding tRNAs rarely used in E. coli, such as ROSETTA™ (DE3) and Rosetta 2 (DE3) strains. Cell lysis and sample handling may also be improved using reagents sold under the trademarks BENZONASE® nuclease and BUGBUSTER® Protein Extraction Reagent. For cell culture, auto-inducing media can improve the efficiency of many expression systems, including high-throughput expression systems. Media of this type (e.g., OVERNIGHT EXPRESS™ Autoinduction System) gradually elicit protein expression through metabolic shift without the addition of artificial inducing agents such as IPTG. Particular embodiments employ hexahistidine tags (such as those sold under the trademark HIS•TAG® fusions), followed by immobilized metal affinity chromatography (IMAC) purification, or related techniques. In certain aspects, however, clinical grade proteins can be isolated from E. coli inclusion bodies, without or without the use of affinity tags (see, e.g., Shimp et al., Protein Expr Purif 50:58-67, 2006). As a further example, certain embodiments may employ a cold-shock induced E. coli high-yield production system, because over-expression of proteins in Escherichia coli at low temperature improves their solubility and stability (see, e.g., Qing et al., Nature Biotechnology. 22:877-882, 2004).

Also included are high-density bacterial fermentation systems. For example, high cell density cultivation of Ralstonia eutropha allows protein production at cell densities of over 150 g/L, and the expression of recombinant proteins at titers exceeding 10 g/L.

In the yeast Saccharomyces cerevisiae, a number of vectors containing constitutive or inducible promoters such as alpha factor, alcohol oxidase, and PGH may be used. For reviews, see Ausubel et al. (supra) and Grant et al., Methods Enzymol. 153:516-544 (1987). Also included are Pichia pandoris expression systems (see, e.g., Li et al., Nature Biotechnology. 24, 210-215, 2006; and Hamilton et al., Science, 301:1244, 2003). Certain embodiments include yeast systems that are engineered to selectively glycosylate proteins, including yeast that have humanized N-glycosylation pathways, among others (see, e.g., Hamilton et al., Science. 313:1441-1443, 2006; Wildt et al., Nature Reviews Microbiol. 3:119-28, 2005; and Gerngross et al., Nature-Biotechnology. 22:1409-1414, 2004; U.S. Pat. Nos. 7,629,163; 7,326,681; and 7,029,872). Merely by way of example, recombinant yeast cultures can be grown in Fernbach Flasks or 15 L, 50 L, 100 L, and 200 L fermentors, among others.

In cases where plant expression vectors are used, the expression of sequences encoding polypeptides may be driven by any of a number of promoters. For example, viral promoters such as the 35S and 19S promoters of CaMV may be used alone or in combination with the omega leader sequence from TMV (Takamatsu, EMBO J. 6:307-311 (1987)). Alternatively, plant promoters such as the small subunit of RUBISCO or heat shock promoters may be used (Coruzzi et al., EMBO J. 3:1671-1680 (1984); Broglie et al., Science 224:838-843 (1984); and Winter et al., Results Probl. Cell Differ. 17:85-105 (1991)). These constructs can be introduced into plant cells by direct DNA transformation or pathogen-mediated transfection. Such techniques are described in a number of generally available reviews (see, e.g., Hobbs in McGraw Hill, Yearbook of Science and Technology, pp. 191-196 (1992)).

An insect system may also be used to express a polypeptide of interest. For example, in one such system, Autographa californica nuclear polyhedrosis virus (AcNPV) is used as a vector to express foreign genes in Spodoptera frugiperda cells or in Trichoplusia cells. The sequences encoding the polypeptide may be cloned into a non-essential region of the virus, such as the polyhedrin gene, and placed under control of the polyhedrin promoter. Successful insertion of the polypeptide-encoding sequence will render the polyhedrin gene inactive and produce recombinant virus lacking coat protein. The recombinant viruses may then be used to infect, for example, S. frugiperda cells or Trichoplusia cells in which the polypeptide of interest may be expressed (Engelhard et al., Proc. Natl. Acad. Sci. U.S.A. 91:3224-3227 (1994)). Also included are baculovirus expression systems, including those that utilize SF9, SF21, and T. ni cells (see, e.g., Murphy and Piwnica-Worms, Curr Protoc Protein Sci. Chapter 5: Unit 5.4, 2001). Insect systems can provide post-translation modifications that are similar to mammalian systems.

In mammalian host cells, a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus which is capable of expressing the polypeptide in infected host cells (Logan & Shenk, Proc. Natl. Acad. Sci. U.S.A. 81:3655-3659 (1984)). In addition, transcription enhancers, such as the Rous sarcoma virus (RSV) enhancer, may be used to increase expression in mammalian host cells.

Examples of useful mammalian host cell lines include monkey kidney CV1 line transformed by SV40 (COS-7, ATCC CRL 1651); human embryonic kidney line (293 or 293 cells sub-cloned for growth in suspension culture, Graham et al., J. Gen Virol. 36:59 (1977)); baby hamster kidney cells (BHK, ATCC CCL 10); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1982)); MRC 5 cells; FS4 cells; and a human hepatoma line (Hep G2). Other useful mammalian host cell lines include Chinese hamster ovary (CHO) cells, including DHFR-CHO cells (Urlaub et al., PNAS USA 77:4216 (1980)); and myeloma cell lines such as NSO and Sp2/0. For a review of certain mammalian host cell lines suitable for protein production, see, e.g., Yazaki and Wu, Methods in Molecular Biology, Vol. 248 (B. K.C Lo, ed., Humana Press, Totowa, N.J., 2003), pp. 255-268. Certain preferred mammalian cell expression systems include CHO and HEK293-cell based expression systems. Mammalian expression systems can utilize attached cell lines, for example, in T-flasks, roller bottles, or cell factories, or suspension cultures, for example, in 1 L and 5 L spinners, 5 L, 14 L, 40 L, 100 L and 200 L stir tank bioreactors, or 20/50 L and 100/200 L WAVE bioreactors, among others known in the art.

Also included is the cell-free expression of proteins. These and related embodiments typically utilize purified RNA polymerase, ribosomes, tRNA and ribonucleotides; these reagents may be produced by extraction from cells or from a cell-based expression system.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf. et al., Results Probl. Cell Differ. 20:125-162 (1994)).

In addition, a host cell strain may be chosen for its ability to modulate the expression of the inserted sequences or to process the expressed protein in the desired fashion. Such modifications of the polypeptide include, but are not limited to, post-translational modifications such as acetylation, carboxylation, glycosylation, phosphorylation, lipidation, and acylation. Post-translational processing which cleaves a “prepro” form of the protein may also be used to facilitate correct insertion, folding and/or function. Different host cells such as yeast, CHO, HeLa, MDCK, HEK293, and W138, in addition to bacterial cells, which have or even lack specific cellular machinery and characteristic mechanisms for such post-translational activities, may be chosen to ensure the correct modification and processing of the foreign protein.

For long-term, high-yield production of recombinant proteins, stable expression is generally preferred. For example, cell lines which stably express a polynucleotide of interest may be transformed using expression vectors which may contain viral origins of replication and/or endogenous expression elements and a selectable marker gene on the same or on a separate vector.

Following the introduction of the vector, cells may be allowed to grow for about 1-2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type. Transient production, such as by transient transfection or infection, can also be employed. Exemplary mammalian expression systems that are suitable for transient production include HEK293 and CHO-based systems.

Any number of selection systems may be used to recover transformed or transduced cell lines. These include, but are not limited to, the herpes simplex virus thymidine kinase (Wigler et al., Cell 11:223-232 (1977)) and adenine phosphoribosyltransferase (Lowy et al., Cell 22:817-823 (1990)) genes which can be employed in tk- or aprt- cells, respectively. Also, antimetabolite, antibiotic or herbicide resistance can be used as the basis for selection; for example, dhfr which confers resistance to methotrexate (Wigler et al., Proc. Natl. Acad. Sci. U.S.A. 77:3567-70 (1980)); npt, which confers resistance to the aminoglycosides, neomycin and G-418 (Colbere-Garapin et al., J. Mol. Biol. 150:1-14 (1981)); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman & Mulligan, Proc. Natl. Acad. Sci. U.S.A. 85:8047-51 (1988)). The use of visible markers has gained popularity with such markers as green fluorescent protein (GFP) and other fluorescent proteins (e.g., RFP, YFP), anthocyanins, β-glucuronidase and its substrate GUS, and luciferase and its substrate luciferin, being widely used not only to identify transformants, but also to quantify the amount of transient or stable protein expression attributable to a specific vector system (see, e.g., Rhodes et al., Methods Mol. Biol. 55:121-131 (1995)).

Also included are high-throughput protein production systems, or micro-production systems. Certain aspects may utilize, for example, hexa-histidine fusion tags for protein expression and purification on metal chelate-modified slide surfaces or MagneHis Ni-Particles (see, e.g., Kwon et al., BMC Biotechnol. 9:72, 2009; and Lin et al., Methods Mol Biol. 498:129-41, 2009)). Also included are high-throughput cell-free protein expression systems (see, e.g., Sitaraman et al., Methods Mol Biol. 498:229-44, 2009).

A variety of protocols for detecting and measuring the expression of polynucleotide-encoded products, using binding agents or antibodies such as polyclonal or monoclonal antibodies specific for the product, are known in the art. Examples include enzyme-linked immunosorbent assay (ELISA), western immunoblots, radioimmunoassays (RIA), and fluorescence activated cell sorting (FACS). These and other assays are described, among other places, in Hampton et al., Serological Methods, a Laboratory Manual (1990) and Maddox et al., J. Exp. Med. 158:1211-1216 (1983).

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used include radionuclides, enzymes, fluorescent, chemiluminescent, or chromogenic agents as well as substrates, cofactors, inhibitors, magnetic particles, and the like.

Host cells transformed with one or more polynucleotide sequences of interest may be cultured under conditions suitable for the expression and recovery of the protein from cell culture. Certain specific embodiments utilize serum free cell expression systems. Examples include HEK293 cells and CHO cells that can grown on serum free medium (see, e.g., Rosser et al., Protein Expr. Purif. 40:237-43, 2005; and U.S. Pat. No. 6,210,922).

An activatable proprotein produced by a recombinant cell may be secreted or contained intracellularly depending on the sequence and/or the vector used. As will be understood by those of skill in the art, expression vectors containing polynucleotides may be designed to contain signal sequences which direct secretion of the encoded polypeptide through a prokaryotic or eukaryotic cell membrane. Other recombinant constructions may be used to join sequences encoding a polypeptide of interest to nucleotide sequence encoding a polypeptide domain which will facilitate purification and/or detection of soluble proteins. Examples of such domains include cleavable and non-cleavable affinity purification and ec50 tags such as avidin, FLAG tags, poly-histidine tags (e.g., 6×His), cMyc tags, V5-tags, glutathione S-transferase (GST) tags, and others.

The protein produced by a recombinant cell can be purified and characterized according to a variety of techniques known in the art. Exemplary systems for performing protein purification and analyzing protein purity include fast protein liquid chromatography (FPLC) (e.g., AKTA and Bio-Rad FPLC systems), high-pressure liquid chromatography (HPLC) (e.g., Beckman and Waters HPLC). Exemplary chemistries for purification include ion exchange chromatography (e.g., Q, S), size exclusion chromatography, salt gradients, affinity purification (e.g., Ni, Co, FLAG, maltose, glutathione, protein A/G), gel filtration, reverse-phase, ceramic HYPERD® ion exchange chromatography, and hydrophobic interaction columns (HIC), among others known in the art. Also included are analytical methods such as SDS-PAGE (e.g., coomassie, silver stain), immunoblot, Bradford, and ELISA, which may be utilized during any step of the production or purification process, typically to measure the purity of the protein composition.

Also included are methods of concentrating activatable proproteins, and composition comprising concentrated soluble activatable proprotein. In some aspects, such concentrated solutions of at least tone activatable proprotein comprise proteins at a concentration of about or at least about 5 mg/mL, 8 mg/mL, 10 mg/mL, 15 mg/mL. 20 mg/mL, or more.

In some aspects, such compositions may be substantially monodisperse, meaning that an activatable proprotein exists primarily (i.e., at least about 90%, or greater) in one apparent molecular weight form when assessed for example, by size exclusion chromatography, dynamic light scattering, or analytical ultracentrifugation.

In some aspects, such compositions have a purity (on a protein basis) of at least about 90%, or in some aspects at least about 95% purity, or in some embodiments, at least 98% purity. Purity may be determined via any routine analytical method as known in the art.

In some aspects, such compositions have a high molecular weight aggregate content of less than about 10%, compared to the total amount of protein present, or in some embodiments such compositions have a high molecular weight aggregate content of less than about 5%, or in some aspects such compositions have a high molecular weight aggregate content of less than about 3%, or in some embodiments a high molecular weight aggregate content of less than about 1%. High molecular weight aggregate content may be determined via a variety of analytical techniques including for example, by size exclusion chromatography, dynamic light scattering, or analytical ultracentrifugation.

Examples of concentration approaches contemplated herein include lyophilization, which is typically employed when the solution contains few soluble components other than the protein of interest. Lyophilization is often performed after HPLC run, and can remove most or all volatile components from the mixture. Also included are ultrafiltration techniques, which typically employ one or more selective permeable membranes to concentrate a protein solution. The membrane allows water and small molecules to pass through and retains the protein; the solution can be forced against the membrane by mechanical pump, gas pressure, or centrifugation, among other techniques.

In certain embodiments, an activatable proprotein in a composition has a purity of at least about 90%, as measured according to routine techniques in the art. In certain embodiments, such as diagnostic compositions or certain pharmaceutical or therapeutic compositions, an activatable proprotein composition has a purity of at least about 95%, or at least about 97% or 98% or 99%. In some embodiments, such as when being used as reference or research reagents, activatable proproteins can be of lesser purity, and may have a purity of at least about 50%, 60%, 70%, or 80%. Purity can be measured overall or in relation to selected components, such as other proteins, e.g., purity on a protein basis.

Purified activatable proproteins can also be characterized according to their biological characteristics. Binding affinity and binding kinetics can be measured according to a variety of techniques known in the art, such as Biacore® and related technologies that utilize surface plasmon resonance (SPR), an optical phenomenon that enables detection of unlabeled interactants in real time. SPR-based biosensors can be used in determination of active concentration, screening and characterization in terms of both affinity and kinetics. The presence or levels of one or more biological activities can be measured according to cell-based assays, including those that utilize at least one IL-15 receptor, which is optionally functionally coupled to a readout or indicator, such as a fluorescent or luminescent indicator of biological activity, as described herein.

In certain embodiments, as noted above, an activatable proprotein composition is substantially endotoxin free, including, for example, about 95% endotoxin free, preferably about 99% endotoxin free, and more preferably about 99.99% endotoxin free. The presence of endotoxins can be detected according to routine techniques in the art, as described herein. In specific embodiments, an activatable proprotein composition is made from a eukaryotic cell such as a mammalian or human cell in substantially serum free media. In certain embodiments, as noted herein, an activatable proprotein composition has an endotoxin content of less than about 10 EU/mg of activatable proprotein, or less than about 5 EU/mg of activatable proprotein, less than about 3 EU/mg of activatable proprotein, or less than about 1 EU/mg of activatable proprotein.

In certain embodiments, an activatable proprotein composition comprises less than about 10% wt/wt high molecular weight aggregates, or less than about 5% wt/wt high molecular weight aggregates, or less than about 2% wt/wt high molecular weight aggregates, or less than about or less than about 1% wt/wt high molecular weight aggregates.

Also included are protein-based analytical assays and methods, which can be used to assess, for example, protein purity, size, solubility, and degree of aggregation, among other characteristics. Protein purity can be assessed a number of ways. For instance, purity can be assessed based on primary structure, higher order structure, size, charge, hydrophobicity, and glycosylation. Examples of methods for assessing primary structure include N- and C-terminal sequencing and peptide-mapping (see, e.g., Allen et al., Biologicals. 24:255-275, 1996)). Examples of methods for assessing higher order structure include circular dichroism (see, e.g., Kelly et al., Biochim Biophys Acta. 1751:119-139, 2005), fluorescent spectroscopy (see, e.g., Meagher et al., J. Biol. Chem. 273:23283-89, 1998), FT-IR, amide hydrogen-deuterium exchange kinetics, differential scanning calorimetry, NMR spectroscopy, immunoreactivity with conformationally sensitive antibodies. Higher order structure can also be assessed as a function of a variety of parameters such as pH, temperature, or added salts. Examples of methods for assessing protein characteristics such as size include analytical ultracentrifugation and size exclusion HPLC (SEC-HPLC), and exemplary methods for measuring charge include ion-exchange chromatography and isoelectric focusing. Hydrophobicity can be assessed, for example, by reverse-phase HPLC and hydrophobic interaction chromatography HPLC. Glycosylation can affect pharmacokinetics (e.g., clearance), conformation or stability, receptor binding, and protein function, and can be assessed, for example, by mass spectrometry and nuclear magnetic resonance (NMR) spectroscopy.

As noted above, certain embodiments include the use of SEC-HPLC to assess protein characteristics such as purity, size (e.g., size homogeneity) or degree of aggregation, and/or to purify proteins, among other uses. SEC, also including gel-filtration chromatography (GFC) and gel-permeation chromatography (GPC), refers to a chromatographic method in which molecules in solution are separated in a porous material based on their size, or more specifically their hydrodynamic volume, diffusion coefficient, and/or surface properties. The process is generally used to separate biological molecules, and to determine molecular weights and molecular weight distributions of polymers. Typically, a biological or protein sample (such as a protein extract produced according to the protein expression methods provided herein and known in the art) is loaded into a selected size-exclusion column with a defined stationary phase (the porous material), preferably a phase that does not interact with the proteins in the sample. In certain aspects, the stationary phase is composed of inert particles packed into a dense three-dimensional matrix within a glass or steel column. The mobile phase can be pure water, an aqueous buffer, an organic solvent, or a mixture thereof. The stationary-phase particles typically have small pores and/or channels which only allow molecules below a certain size to enter. Large particles are therefore excluded from these pores and channels, and their limited interaction with the stationary phase leads them to elute as a “totally-excluded” peak at the beginning of the experiment. Smaller molecules, which can fit into the pores, are removed from the flowing mobile phase, and the time they spend immobilized in the stationary-phase pores depends, in part, on how far into the pores they penetrate. Their removal from the mobile phase flow causes them to take longer to elute from the column and results in a separation between the particles based on differences in their size. A given size exclusion column has a range of molecular weights that can be separated. Overall, molecules larger than the upper limit will not be trapped by the stationary phase, molecules smaller than the lower limit will completely enter the solid phase and elute as a single band, and molecules within the range will elute at different rates, defined by their properties such as hydrodynamic volume. For examples of these methods in practice with pharmaceutical proteins, see Bruner et al., Journal of Pharmaceutical and Biomedical Analysis. 15: 1929-1935, 1997.

Protein purity for clinical applications is also discussed, for example, by Anicetti et al. (Trends in Biotechnology. 7:342-349, 1989). More recent techniques for analyzing protein purity include, without limitation, the LabChip GXII, an automated platform for rapid analysis of proteins and nucleic acids, which provides high throughput analysis of titer, sizing, and purity analysis of proteins. In certain non-limiting embodiments, clinical grade activatable proproteins can be obtained by utilizing a combination of chromatographic materials in at least two orthogonal steps, among other methods (see, e.g., Therapeutic Proteins: Methods and Protocols. Vol. 308, Eds., Smales and James, Humana Press Inc., 2005). Typically, protein agents (e.g., activatable proprotein) are substantially endotoxin-free, as measured according to techniques known in the art and described herein.

Protein solubility assays are also included. Such assays can be utilized, for example, to determine optimal growth and purification conditions for recombinant production, to optimize the choice of buffer(s), and to optimize the choice of activatable proproteins and variants thereof. Solubility or aggregation can be evaluated according to a variety of parameters, including temperature, pH, salts, and the presence or absence of other additives. Examples of solubility screening assays include, without limitation, microplate-based methods of measuring protein solubility using turbidity or other measure as an end point, high-throughput assays for analysis of the solubility of purified recombinant proteins (see, e.g., Stenvall et al., Biochim Biophys Acta. 1752:6-10, 2005), assays that use structural complementation of a genetic marker protein to monitor and measure protein folding and solubility in vivo (see, e.g., Wigley et al., Nature Biotechnology. 19:131-136, 2001), and electrochemical screening of recombinant protein solubility in Escherichia coli using scanning electrochemical microscopy (SECM) (see, e.g., Nagamine et al., Biotechnology and Bioengineering. 96:1008-1013, 2006), among others. Activatable proprotein with increased solubility (or reduced aggregation) can be identified or selected for according to routine techniques in the art, including simple in vivo assays for protein solubility (see, e.g., Maxwell et al., Protein Sci. 8:1908-11, 1999).

Protein solubility and aggregation can also be measured by dynamic light scattering techniques. Aggregation is a general term that encompasses several types of interactions or characteristics, including soluble/insoluble, covalent/noncovalent, reversible/irreversible, and native/denatured interactions and characteristics. For protein therapeutics, the presence of aggregates is typically considered undesirable because of the concern that aggregates may cause an immunogenic reaction (e.g., small aggregates), or may cause adverse events on administration (e.g., particulates). Dynamic light scattering refers to a technique that can be used to determine the size distribution profile of small particles in suspension or polymers such as proteins in solution. This technique, also referred to as photon correlation spectroscopy (PCS) or quasi-elastic light scattering (QELS), uses scattered light to measure the rate of diffusion of the protein particles. Fluctuations of the scattering intensity can be observed due to the Brownian motion of the molecules and particles in solution. This motion data can be conventionally processed to derive a size distribution for the sample, wherein the size is given by the Stokes radius or hydrodynamic radius of the protein particle. The hydrodynamic size depends on both mass and shape (conformation). Dynamic scattering can detect the presence of very small amounts of aggregated protein (<0.01% by weight), even in samples that contain a large range of masses. It can also be used to compare the stability of different formulations, including, for example, applications that rely on real-time monitoring of changes at elevated temperatures. Accordingly, certain embodiments include the use of dynamic light scattering to analyze the solubility and/or presence of aggregates in a sample that contains an activatable proprotein of the present disclosure.

Although the foregoing embodiments have been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### Examples

### Example 1A. Preparation of PD-L1-proIL-15 Fusion Proteins

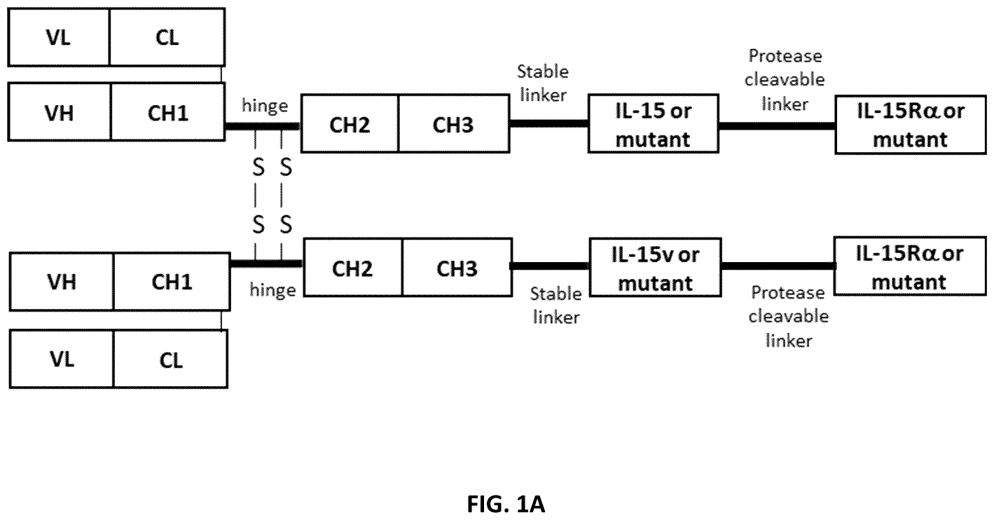

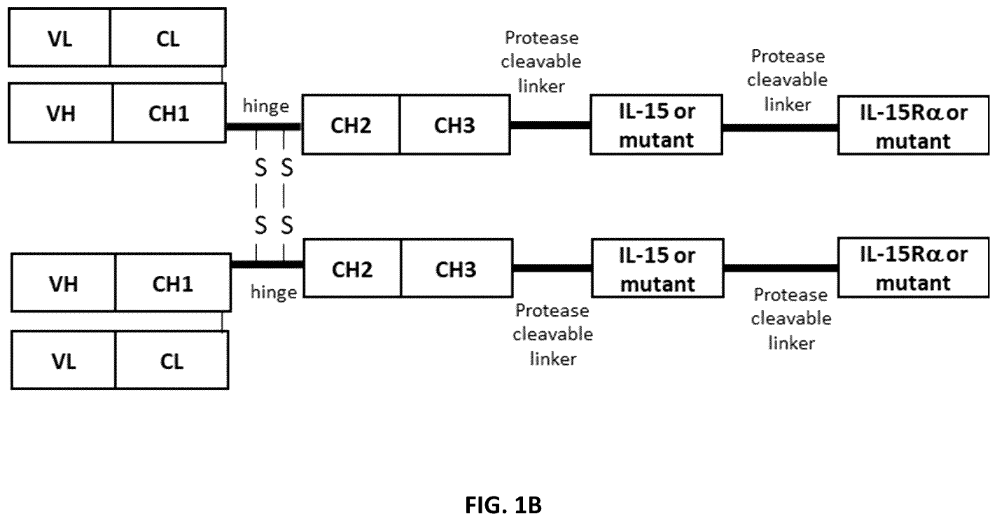

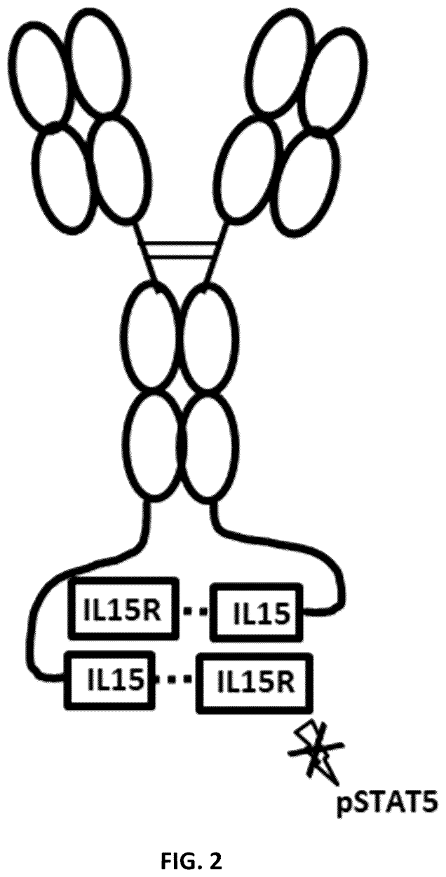

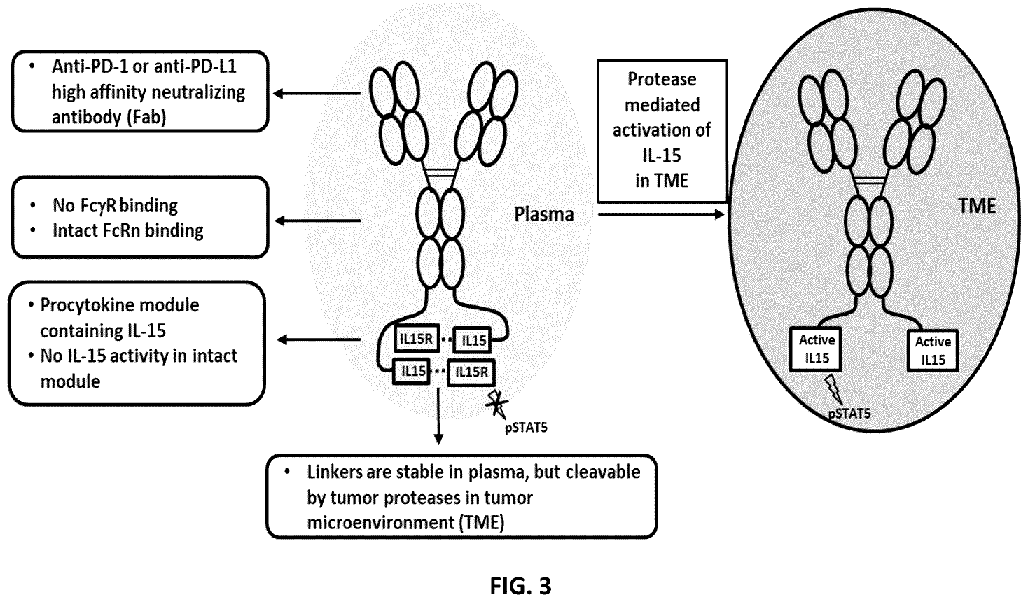

Plasmids coding for PD-L1-proIL-15 or anti-human PD-L1 were constructed by standard gene synthesis, followed by sub-cloning into pTT5 expression vector. Schematics of illustrative IgG-proIL-15 fusion protein formats are depicted in FIGS. 1-2.

Illustrative proteins of PD-L1-proIL-15 format include P53021942 (SEQ ID NOs: 144 and 145). Illustrative proteins of anti-human PD-L1 include P40751942 (SEQ ID NOs: 140 and 141).

PD-L1-proIL-15 fusion proteins or anti-human PD-L1 were produced by transient transfection in Expi293 cells and purified by one-step purification of MabSelect SuRe chromatography (GE Healthcare). Purified proteins were characterized by SDS-PAGE and high performance liquid chromatography (HPLC) for purity and homogeneity assessment. HPLC analysis was performed using Nanofilm SEC-250 column (Sepax) and Agilent 1260 according to the manufacturer's instructions. The purified proteins showed high purity on SDS-PAGE gel and good homogeneity based on HPLC results.

### Example 1B. Preparation of PD-1-proIL-15 Fusion Proteins

Plasmids coding for PD-1-proIL-15 or anti-human PD-1 were constructed by standard gene synthesis, followed by sub-cloning into pTT5 expression vector. Schematics of illustrative IgG-proIL-15 fusion protein formats are depicted in FIGS. 1-2.

Illustrative proteins of PD-1-proIL-15 format include P53052037 (SEQ ID NOs: 146 and 147) and P79772037 (SEQ ID NOs: 199 and 200). Illustrative proteins of murine surrogate mPD-1-proIL-15 format include P55654367 (SEQ ID NOs: 148 and 149). Illustrative proteins of anti-human PD-1 include P42412037 (SEQ ID NOs: 142 and 143).

PD-1-proIL-15 fusion proteins or anti-human PD-1 were produced by transient transfection in Expi293 cells and purified by one-step purification of MabSelect SuRe chromatography (GE Healthcare). Purified proteins were characterized by SDS-PAGE and high performance liquid chromatography (HPLC) for purity and homogeneity assessment. HPLC analysis was performed using Nanofilm SEC-250 column (Sepax) and Agilent 1260 according to the manufacturer's instructions. The purified proteins showed high purity on SDS-PAGE gel and good homogeneity based on HPLC results.

### Example 1C. Preparation of B7H3-proIL-15 Fusion Proteins

Plasmids coding for B7H3-proIL-15 with different protease cleavable linkers were constructed by standard gene synthesis, followed by sub-cloning into pTT5 expression vector.

Schematics of illustrative IgG-proIL-15 fusion protein formats are depicted in FIGS. 1-2.

Illustrative proteins of B7H3-proIL-15 format with different protease cleavage linkers include P40503699 (SEQ ID NOs: 136 and 137), P40743699 (SEQ ID NOs: 138 and 139).

B7H3-proIL-15 fusion proteins were produced by transient transfection in Expi293 cells and purified by one-step purification of MabSelect SuRe chromatography (GE Healthcare). Purified proteins were characterized by SDS-PAGE and high performance liquid chromatography (HPLC) for purity and homogeneity assessment. HPLC analysis was performed using Nanofilm SEC-250 column (Sepax) and Agilent 1260 according to the manufacturer's instructions. The purified proteins showed high purity on SDS-PAGE gel and good homogeneity based on HPLC results.

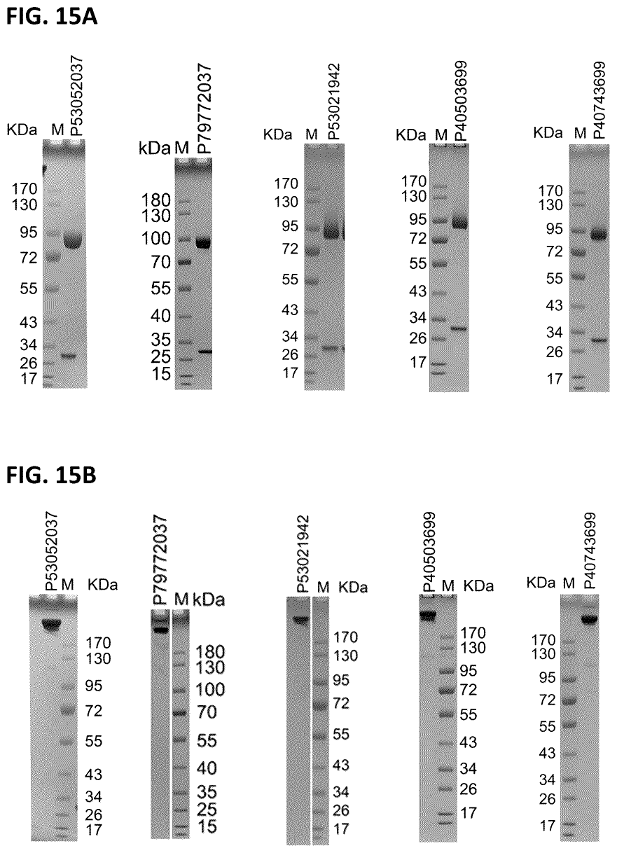

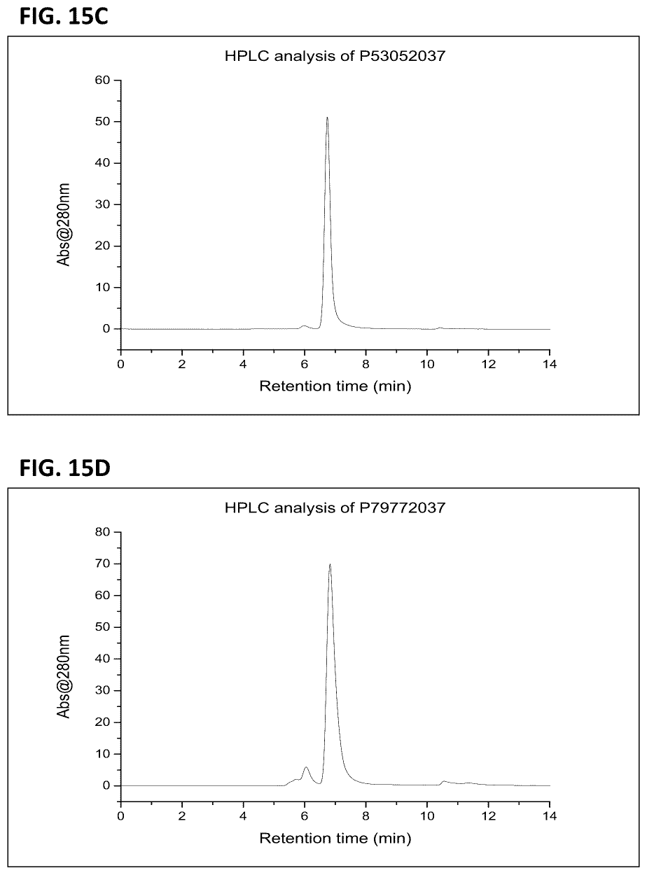

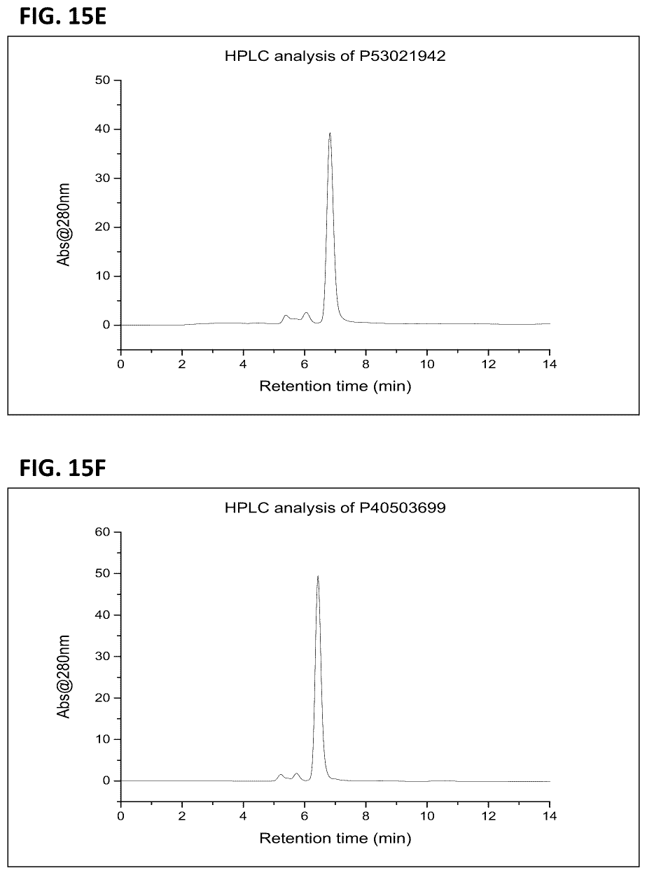

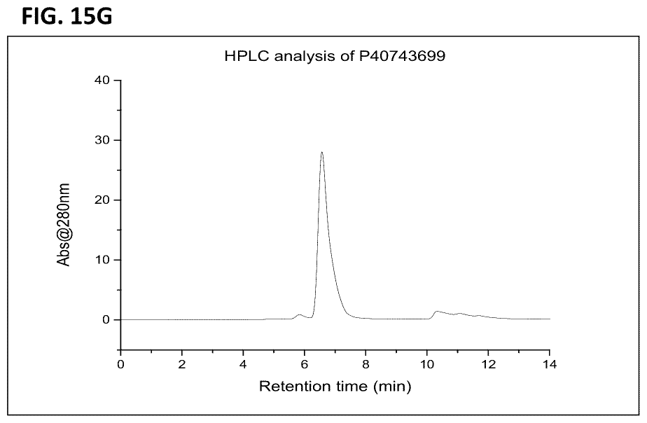

FIGS. 15A-15G show the purification and characterization of PD-1-proIL-15 (P53052037, P79772037), PD-L1-proIL-15 (P53021942), and B7H3-proIL-15 (P40503699, P40743699). FIG. 15A shows the reduced SDS-PAGE analysis results, and FIG. 15B shows the non-reduced SDS-PAGE analysis results. FIGS. 15C-15G shows the size exclusion chromatography (SEC-HPLC) analysis results indicating high purity and homogeneity of products (without significant amount of aggregation or degradation product).

### Example 2A. Binding of PD-1-proIL-15 to Human PD-1

The binding activity of P53052037 and P42412037 was determined by ELISA. Microtitre plates were coated with 100 ul 2 ug/ml of Streptavidin overnight at 4° C. The next day, plates were washed with PBS and blocked with 2% BSA (2% Bovine Serum Album in PBS). 100 ul 2 ug/ml of biotinylated huPD-1 was added into corresponding wells and incubated for 1 hr at room temperature to capture biotinylated protein. The plates were washed with PBST (0.01% Tween in PBS) and PBS sequentially.

The indicated samples (PD-1 IgG or PD-1-proIL-15) used in ELISA assay were prepared in 2% BSA to an initial concentration of 10 ug/ml, followed by ⅓ serial dilutions. 100 μl diluted protein was added into corresponding wells and incubated at room temperature for 1 h. Plates were washed with PBST and PBS sequentially.

Bound antibodies were detected with peroxidase-conjugated anti-human IgG secondary antibody (Jackson Immunoresearch). Plates were washed with PBST and PBS sequentially. 90 ul of TMB substrate was added into each well and incubated in dark at room temperature for 5 min. The reaction was terminated with 45 ul 2M sulphuric acid and the absorbance was read at 450 nm. The data were analyzed by Prism.

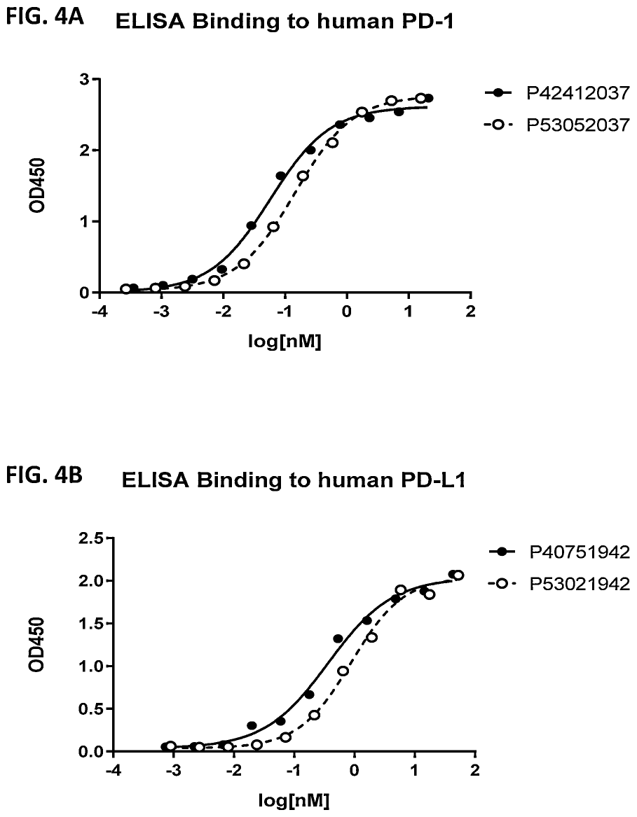

As shown in FIG. 4A, PD-1-proIL-15 and the corresponding PD-1 IgG bind similarly to human PD-1.

### Example 2B. Binding of PD-L1-proIL-15 to Human PD-L1

The binding activity of P53021942 and P40751942 was determined by ELISA. Microtitre plates were coated with 100 ul 2 ug/ml of Streptavidin overnight at 4° C. The next day, plates were washed with PBS and blocked with 2% BSA (2% Bovine Serum Album in PBS). 100 ul 2 ug/ml of biotinylated huPD-L1 was added into corresponding wells and incubated for 1 hr at room temperature to capture biotinylated protein. The plates were washed with PBST (0.01% Tween in PBS) and PBS sequentially.

The indicated samples (PD-L1 IgG or PD-L1-proIL-15) used in ELISA assay were prepared in 2% BSA to an initial concentration of 10 ug/ml, followed by ⅓ serial dilutions. 1001 diluted protein was added into corresponding wells and incubated at room temperature for 1 h. Plates were washed with PBST and PBS sequentially.

Bound antibodies were detected with peroxidase-conjugated anti-human IgG secondary antibody (Jackson Immunoresearch). Plates were washed with PBST and PBS sequentially. 90 ul of TMB substrate was added into each well and incubated in dark at room temperature for 5 min. The reaction was terminated with 45 ul 2M sulphuric acid and the absorbance was read at 450 nm. The data were analyzed by Prism.

As shown in FIG. 4B, PD-L1-proIL-15 and the corresponding PD-L1 IgG bind similarly to human PD-L1.

### Example 2C. Proliferation of M07-e with PD-1-proIL-15 and PD-L1-proIL-15

Human M-07e cells (expressing human IL-15β/γ) were cultured in RPMI 1640 supplemented with 20% fetal bovine serum (FBS) and 10% of 5637 cell culture supernatant. To measure cytokine-dependent cell proliferation, M-07e cells were harvested in their logarithmic growth phase and washed twice with PBS. 90 μl of cell suspension (2×10⁴ cells/well) was seeded into 96-well plate and incubated for 4 hours in assay medium (RPMI 1640 supplemented with 10% FBS) for cytokine starvation at 37° C. and 5% CO2. IL-15, P53052037 or P53021942 protein samples used in assays were prepared in assay medium to an initial concentration (30 nM for IL-15, 810 nM for P53052037/P53021942 and 270 nM for MMP2 activated fusion proteins), followed by ⅓ serial dilutions. 10 μl diluted protein was added into corresponding wells and incubated at 37° C. and 5% CO2 for 72 hours. Colorimetric assays using a Cell Counting Kit-8 (CCK-8, Dojindo, CK04) were performed to measure the amount of live cells.

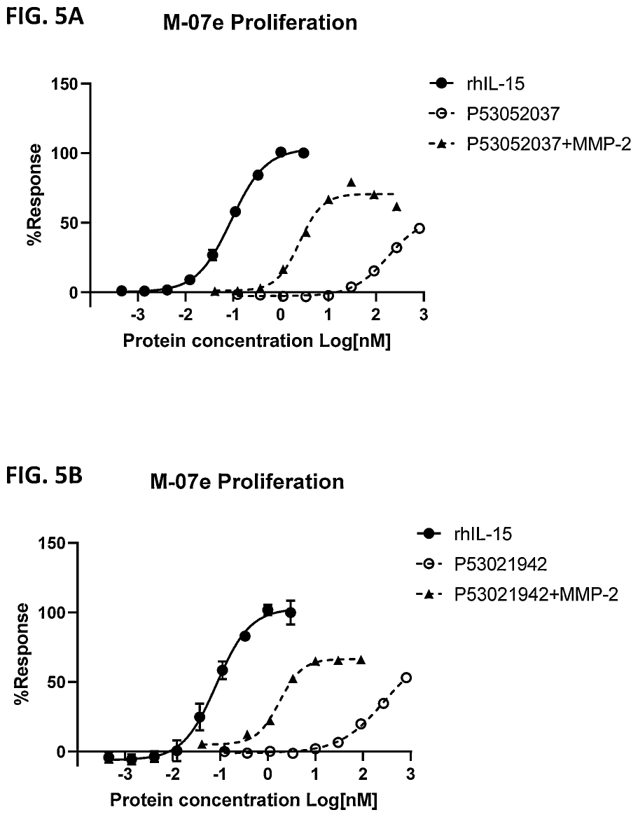

FIG. 5A shows that PD-1-proIL-15 is not able to induce proliferation of M07-e even at the highest concentration. MMP-2 cleaved PD-1-proIL-15 restored partial IL-15 activity and exhibited ˜20 fold lower activity compared with wild type IL-15. FIG. 5B shows that PD-L1-proIL-15 has almost no activity at the highest concentration of 810 nM. In contrast, MMP-2 cleaved PD-L1-proIL-15 induced proliferation of M07-e cells as efficiently as PD-1-proIL-15.

### Example 3A. Cell Activation of Resting and Activated PBMC with PD-1-proIL-15 (pSTAT5 Assay

Frozen human PBMCs (SAILYBIO, donor 1) were recovered in RPMI-1640 added with 10% FBS, 100 U/ml penicillin and 100 μg/ml streptomycin at 37° C. in an atmosphere of 5% CO2 for ˜2 hours. For assays performed in pre-activated PBMC, a 10-cm plate was coated with αCD3 (1 μg/ml, BioLegend) overnight at 4° C. The recovered PBMCs were suspended in RPMI1640 with addition of soluble αCD28 (1 μg/ml, BioLegend) and cultured for 3 Days at 37° C. in an atmosphere of 5% CO2. Resting or pre-activated PBMCs were adjusted to 2.6×10⁶ cells/ml, and plated at 4×105 cells per well into 96-well plate. After incubation for 15 min at 37° C. with rhIL-15 or P53052037, cells were immediately fixed with Cytofix buffer (BD Bioscience) to preserve the phosphorylation status for 15 min on ice and wash once with BD Pharmingen™ Stain Buffer (FBS).

For surface staining, cells were incubated with CD3 Alexa Flour 700 (BD 557943), CD4 PerCP-Cytm5.5(BD 560650), CD8 APC-Cytm7 (BD 557760), CD25 BV421(BD562442), CD56 BV510 (BD 744218) for 30 min at 4° C. The cells were washed once with 1×PBS and centrifuged at 500 g for 5 min and the supernatant was removed by aspiration. The cells were permeabilized with pre-cold Phosflow Perm buffer III (BD Bioscience) for 30 min at 4° C. Before beginning the intracellular staining, the cells were washed once with 1×PBS and centrifuged at 500 g for 5 min to collect the pellet. The cells were then stained with Foxp3 PE (BD 560046) and Anti-Stat5 (pY694) Alexa Fluor® 647 (BD 562076) for 40 min at RT. The cells were washed twice with BD Pharmingen™ Stain Buffer (FBS) and centrifuged at 500 g for 5 min to pellet the cells and to remove the supernatant. The cell pellet was resuspended in 200 ul (per well) BD Pharmingen Stain Buffer (FBS) and analyzed by flow cytometry. STAT5 phosphorylation status in PBMC subsets upon pro-drug treatments was acquired and processed by CytoFLEX (Beckman).

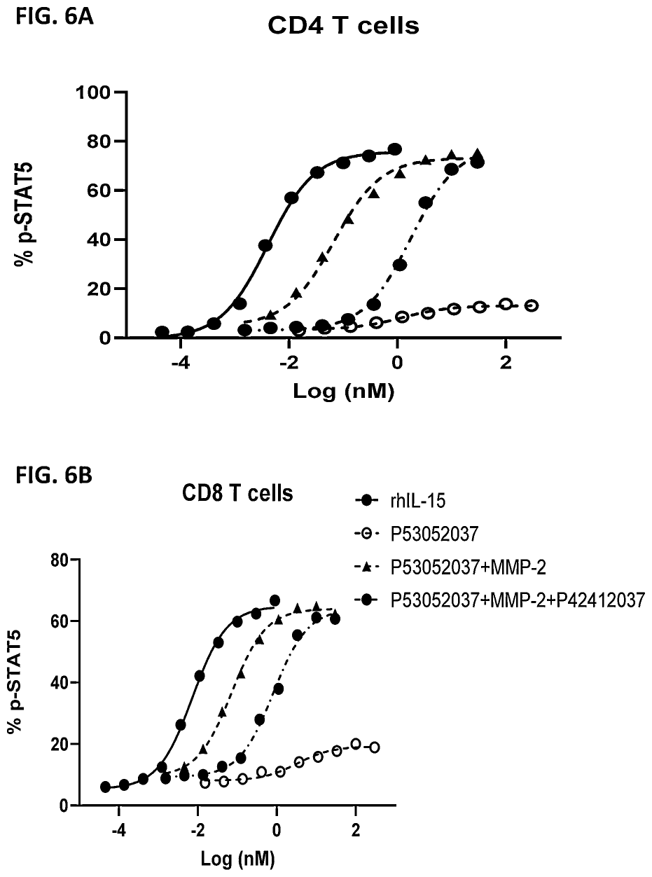

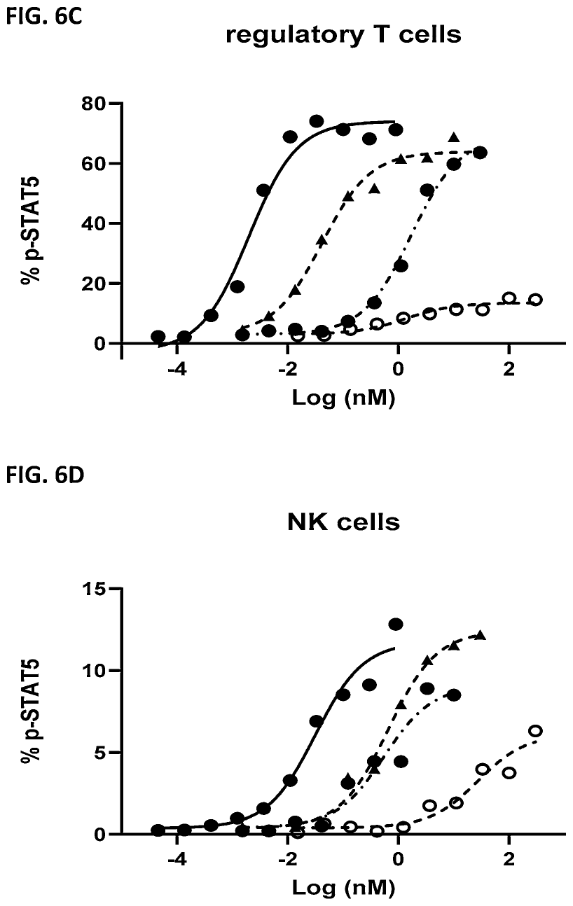

FIGS. 6A-6D show STAT5 phosphorylation in CD4 T-cells (6A), CD8 T-cells (6B), regulatory T-cells (6C), and NK cells (6D) upon treatment of resting PBMCs of donor 1 with rhIL-15 as well as intact and protease activated PD-1-proIL-15. The masked or inactive procytokine form of P53052037 was unable to induce STAT5 phosphorylation in all tested cell subsets. The protease activated form of P53052037 (Matrix metalloproteinase-2 cleaved) was equally effective in activating STAT5 phosphorylation in CD8 and CD4 T cells and less potent in NK cells. Notably, pre-blocking of PD-1 receptor by the parental anti-PD-1 antibody reduced the P53052037 (activated form) potency over 20-fold on PD-1 expressed T cells. These data validate that preferential cis-targeting of PD-1-IL-15 results in significantly enhanced potency on PD-1 expressing T cells.

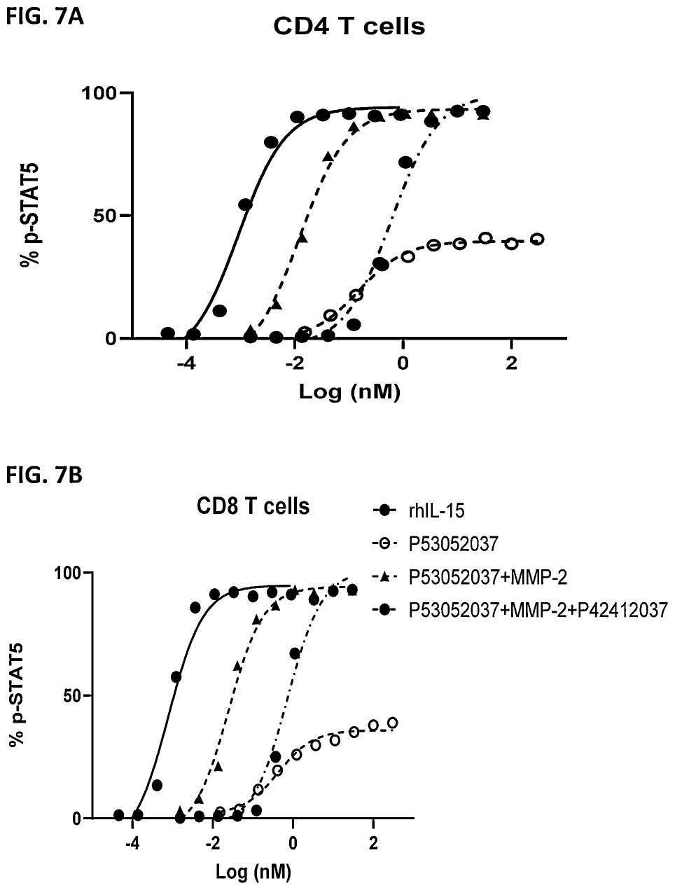

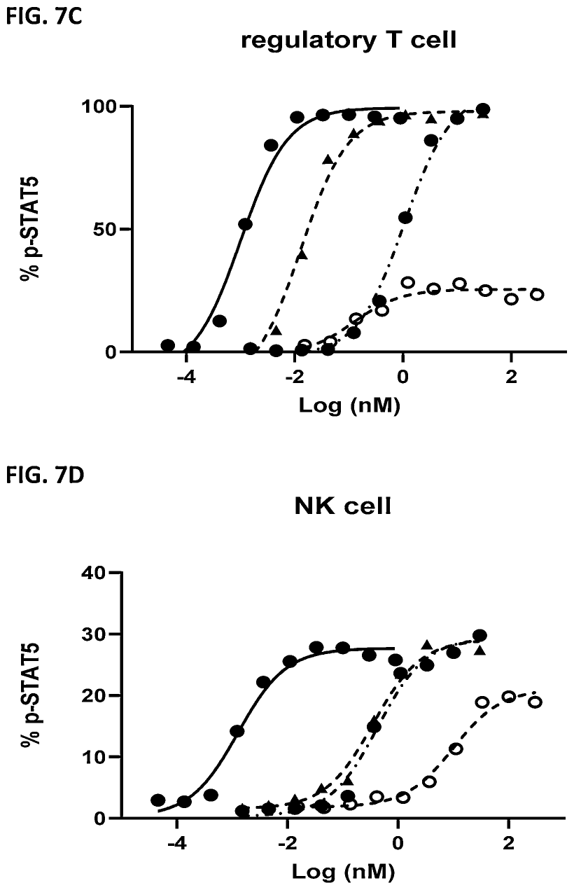

FIGS. 7A-7D show STAT5 phosphorylation in CD4 T-cells (7A), CD8 T-cells (7B), regulatory T-cells (7C) and NK cells (7D) upon pre-activated PBMCs of donor 1. The data confirms that PD-1 binding enhances P53052037 (PD-1-proIL-15 fusion protein) potency.

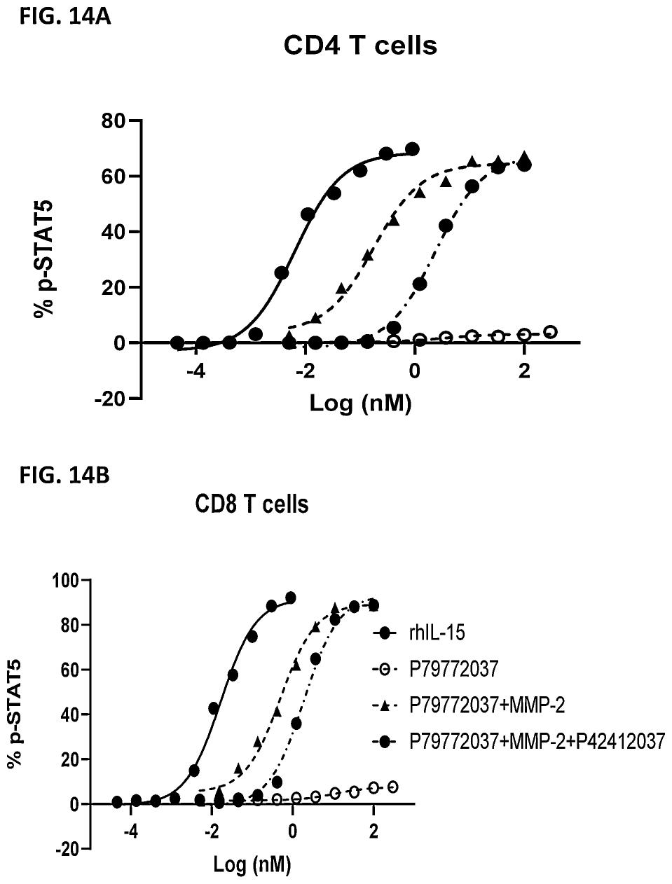

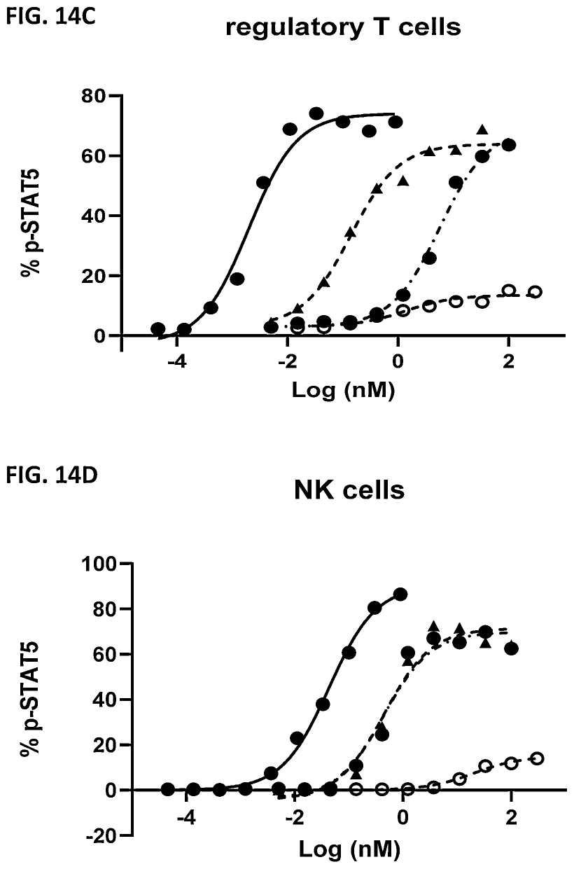

FIGS. 14A-14D show STAT5 phosphorylation in CD4 T-cells (14A), CD8 T-cells (14B), regulatory T-cells (14C) and NK cells (14D) upon treatment of resting PBMCs with rhIL-15 as well as intact and activated PD-1-proIL-15. The proprotein form of P79772037 was unable to induce STAT5 phosphorylation in all tested cell subsets. The activated form of P79772037 (Matrix metalloproteinase-2 cleaved) was equally effective in activating STAT5 phosphorylation in CD8 and CD4 T cells and less potent in NK cells. Similar to P53052037, pre-blocking of the PD-1 receptor by the parental anti-PD1 antibody reduced the P79772037 (activated form) potency on PD-1 expressing T cells, evidencing that cis-binding of P79772037 to PD-1 and IL-2βγ on the same cell surface significantly enhanced P79772037 potency.

### Example 3B. Cell Activation of Resting PBMCs with PD-L1-proIL-15 (pSTAT5 Assay

Frozen human PBMCs (SAILYBIO, donor 2) were recovered in RPMI-1640 added with 10% FBS, 100 U/ml penicillin and 100 μg/ml streptomycin at 37° C. in an atmosphere of 5% CO2 for ˜2 hours. PBMCs were adjusted to 2.6×10⁶ cells/ml, and plated at 4×10⁵ cells per well into 96-well plate. The recovered PBMCs were incubated for 15 min at 37° C. with rhIL-15 and P53021942 (both intact and activated forms). After incubation, cells were immediately fixed with Cytofix buffer (BD Bioscience) to preserve the phosphorylation status for 15 min on ice and wash once with BD Pharmingen™ Stain Buffer (FBS).

For surface staining, cells were incubated with CD3 Alexa Flour 700 (BD 557943), CD4 PerCP-Cytm5.5(BD 560650), CD8 APC-Cytm7 (BD 557760), CD25 BV421(BD562442), CD56 BV510 (BD 744218) for 30 min at 4° C. The cells were washed once with 1×PBS and centrifuged at 500 g for 5 min and the supernatant was removed by aspiration. The cells were permeabilized with pre-cold Phosflow Perm buffer III (BD Bioscience) for 30 min at 4° C. Before beginning the intracellular staining, the cells were washed once with 1×PBS and centrifuged at 500 g for 5 min to collect the pellet. The cells were then stained with Foxp3 PE (BD 560046) and Anti-Stat5 (pY694) Alexa Fluor® 647 (BD 562076) for 40 min at RT. The cells were washed twice with BD Pharmingen™ Stain Buffer (FBS) and centrifuged at 500 g for 5 min to pellet the cells and to remove the supernatant. The cell pellet was resuspended in 200 ul (per well) BD Pharmingen Stain Buffer (FBS) and analyzed by flow cytometry. STAT5 phosphorylation status in PBMC subsets upon procytokine treatments was acquired and processed by CytoFLEX (Beckman).

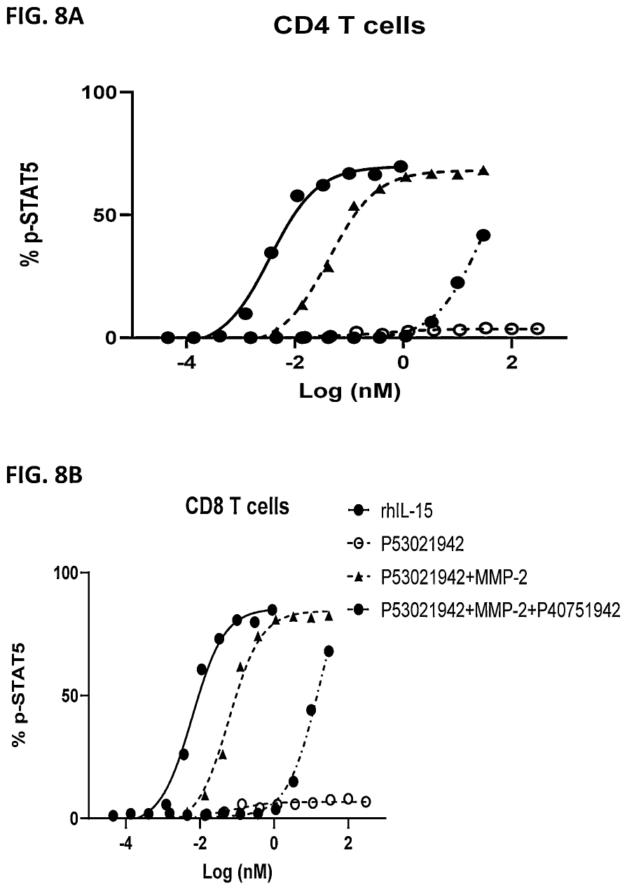

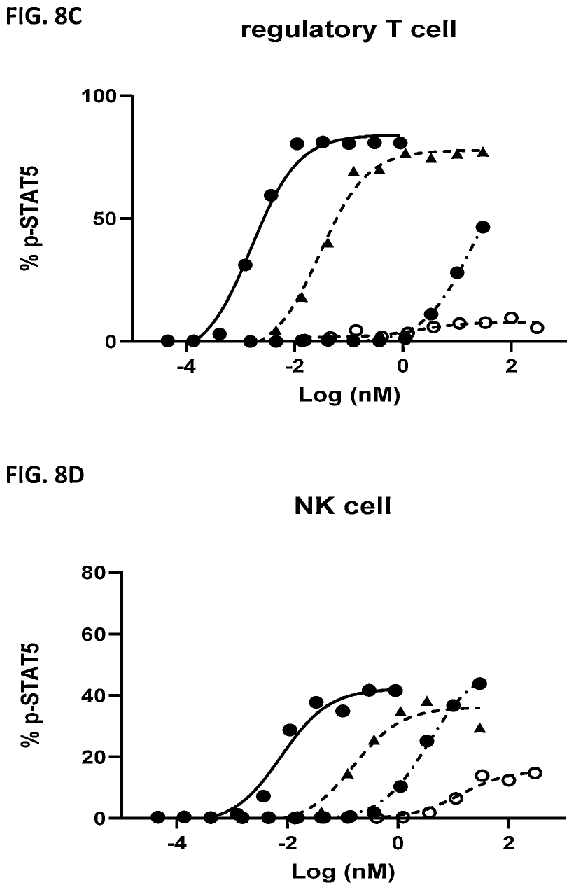

FIGS. 8A-8D show STAT5 phosphorylation in CD4 T-cells (8A), CD8 T-cells (8B), regulatory T-cells (8C) and NK cells (8D) upon treatment of resting PBMCs of donor 2 with rhIL-15 as well as intact and protease activated PD-L1-proIL-15. The masked or inactive procytokine form of P53021942 was unable to induce STAT5 phosphorylation in all tested cell subsets. The activated form of P53021942 (Matrix metalloproteinase-2 cleaved) was equally effective in activating STAT5 phosphorylation in CD8 and CD4 T cells and less potent in NK cells. Notably, pre-blocking of PD-L1 receptor by the parental anti-PD-L1 antibody reduced the P53021942 (activated form) potency over 100-fold on PD-L1 expressed CD4+T, CD8+T and Tregs cells. These data validate that PD-L1 binding enhances IL-15 potency.

### Example 3C. Activated PD-1/L1-proIL-15 Stimulates IFNγ Secretion by PBMCs

Frozen PBMCs of donor 1 were recovered with RPMI1640 with addition of soluble 1 μg/ml αCD3 and were plated into 96-well plate at 4e5 cells/well with increasing concentrations of parental PD-1/L1 antibody or PD-1/PD-L1-proIL-15 fusion protein (both intact and activated forms). After 3 days incubation at 37° C., IFNγ secretion in culture supernatants was analyzed by Human IFN-γ ELISA Set (Biolegend Cat.430104). The pre-coated Capture Antibody plate was prepared as described in the manufacturer's instructions and 100 ul of diluted standard or sample was added into corresponding well and incubated at room temperature for 2 h. Plates were washed 4 times with Wash Buffer, and 100 ul of diluted Detection Antibody solution was added to each well and incubate for 30 min at room temperature with shaking. Plates were then washed 5 times with Wash Buffer, and 100 ul of freshly mixed TMB Substrate solution was added into each well and incubated in dark at room temperature for 20 min. The reaction was terminated with 100 ul of Stop Solution and the absorbance was read at 450 nm. The data were analyzed by Prism.

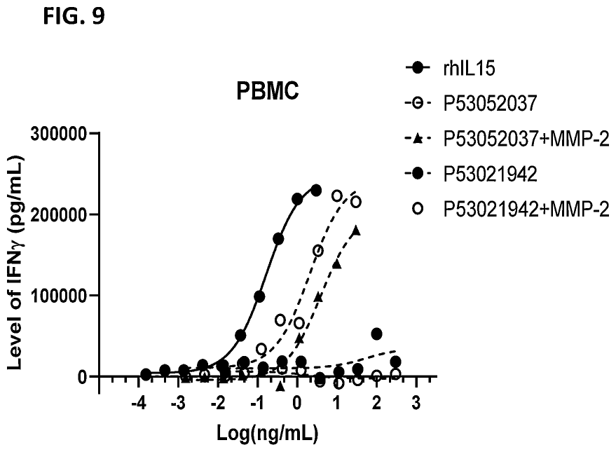

As shown in FIG. 9, activated PD-1/L1-proIL-15 but not the intact procytokine stimulates production of IFNγ by PBMCs. In contrast, PD-1/L1 blockade alone did not induce any significant level of IFNγ secretion.

### Example 4. In Vivo Efficacy of B7H3-proIL-15 with Different Protease Cleavable Linkers in A375-PBMC Xenograft Models

The B7H3-proIL-15 with different protease cleavable linkers were tested as single agents for their anti-tumor efficacy in the A375-PBMC xenograft model.

The A375 cells were maintained in vitro in DMEM added with 10% FBS, 100 U/ml penicillin and 100 μg/ml streptomycin at 37° C. in an atmosphere of 5% CO₂ in air. The human PBMCs were co-cultured with mitomycin C treated A375 tumor cells for 6 days, which maintained in vitro as a suspension cultured in RPMI-1640 added with 10% FBS, 100 U/ml penicillin and 100 μg/ml streptomycin at 37° C. in an atmosphere of 5% CO₂ in air.

Female NCG mice aged 8˜10 weeks at the start of the experiment (GemPharmatech Co., Ltd., Nanjing, China) were maintained under specific-pathogen-free condition with daily cycles of 12 h light/12 h darkness. The mice were kept in individual ventilation cages at constant temperature (20-26° C.) and humidity (40-70%) with ≤6 animals in each cage. Animals had free access to irradiation sterilized dry granule food and sterile drinking water during the entire study period. All the procedures related to animal handling, care and the treatment in this study were performed according to the guidelines approved by the Institutional Animal Care and Use Committee (IACUC) of Shanghai Viva prior to conduct. After arrival, animals were maintained for at least 3 days to get accustomed to the new environment. At the time of routine monitoring, the animals were checked for any effects of tumor growth on normal behavior such as mobility, food and water consumption (by looking only), body weight gain/loss (body weights were measured twice weekly), eye/hair matting and any other abnormal effect.

Mice were inoculated subcutaneously at the right flank with 4×10⁶ A375 cells and 4×10⁵ hPBMCs (co-cultured with A375) in 0.2 ml HBSS contained 0.1 ml matrigel (1:1) for tumor development. One week after the tumor cell inoculation, mice were injected i.v. with B7H3-proIL-15s. The mice in the vehicle group were injected with PBS. Tumor volumes were measured in two dimensions using a caliper, and the volumes were expressed in mm³ using the formula: V=0.5a×b² where a and b are the longest and shortest diameters of the tumor, respectively. The tumor volume was then used for calculations TGI and T/C values. Tumor growth inhibition (TGI %) and relative tumor growth inhibition (T/C %) was calculated according to the following equation:

TGI%=(1−(Tₙ−T₀)/(Vₙ−V₀))×100% T/C%=(Tₙ/T₀)/(Vₙ/V₀)×100%

In the formula, Tₙ and Vₙ stands for tumor volume of treatment group and vehicle control group of day n after the start of treatment, respectively. T₀ and V₀ stands for tumor volume of corresponding groups on the day of grouping. Results were analyzed using Prism GraphPad.

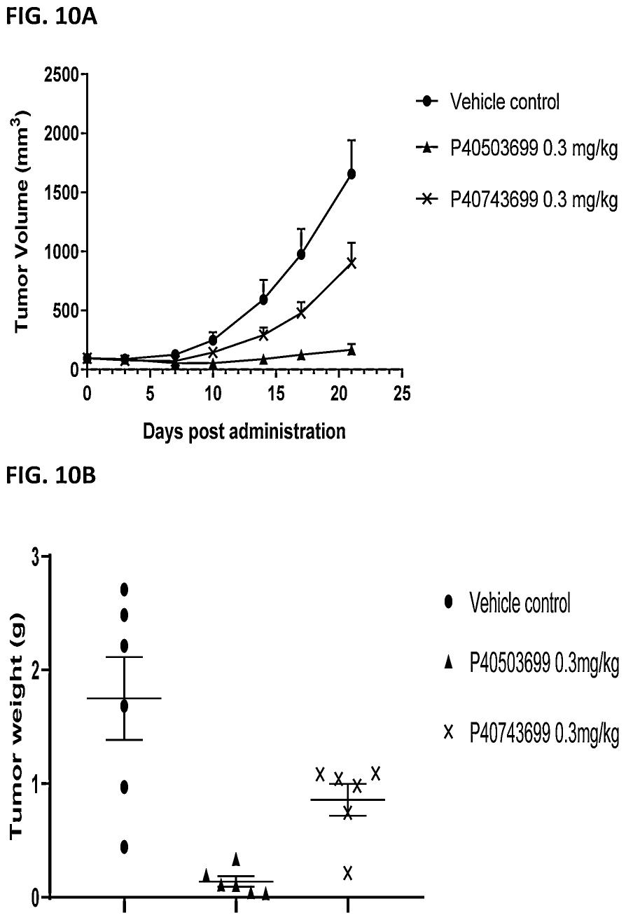

As the protease cleavable linkers in P40503699 are easier to be cleaved than that of in P40743699, FIGS. 10A-10B and Table 1 show that in A375-PBMC xenograft model, P40503699 shows a better anti-tumor activity than that of P40743699 at 0.3 mg/kg. ⅙ of the mice treated with P40503699 show the complete responses.

TABLE 1
Tumor volume and tumor weight of the mice treated with B7H3-
proIL-15 with different linkers in A375-PBMC xenograft model

| | | Tumor | | | Tumor |
|---|---|---|---|---|---|
| | Animal | volume (mm³) | T/C | TGI | weight (g) |
| Treatment | No. | at day 21 | (%) | (%) | at day 22 |
| Vehicle control | 6 | 1656 ± 285 | — | — | 1.750 ± 0.365 |
| P40503699 0.3 | 6 | 169 ± 46 | 10.21 | 95.32 | 0.140 ± 0.046 |
| mg/kg | | | | | |
| P40743699 0.3 | 6 | 902 ± 172 | 54.47 | 48.33 | 0.859 ± 0.139 |
| mg/kg | | | | | |

### Example 5. In Vivo Efficacy of PD-L1-proIL-15 and PD-L1-proIL-15 in A375-PBMC Xenograft Model

The PD-L1-proIL-15 and PD-1-proIL-15 were tested for their anti-tumor efficacy in the A375-PBMC xenograft models. The A375 cells were maintained in vitro in DMEM added with 10% FBS, 100 U/ml penicillin and 100 μg/ml streptomycin at 37° C. in an atmosphere of 5% CO₂ in air.

Female NCG mice aged 8-10 weeks at the start of the experiment (GemPharmatech Co., Ltd., Nanjing, China) were maintained under specific-pathogen-free condition with daily cycles of 12 h light/12 h darkness. The mice were kept in individual ventilation cages at constant temperature (20-26° C.) and humidity (40-70%) with ≤6 animals in each cage. Animals had free access to irradiation sterilized dry granule food and sterile drinking water during the entire study period. All the procedures related to animal handling, care and the treatment in this study were performed according to the guidelines approved by the Institutional Animal Care and Use Committee (IACUC) of Shanghai Viva prior to conduct. After arrival, animals were maintained for at least 3 days to get accustomed to the new environment. At the time of routine monitoring, the animals were checked for any effects of tumor growth on normal behavior such as mobility, food and water consumption (by looking only), body weight gain/loss (body weights were measured twice weekly), eye/hair matting and any other abnormal effect.

Mice were inoculated subcutaneously at the right flank with 3.5×10⁶ A375 cells and 3.5×10⁵ hPBMCs in 0.2 ml HBSS contained 0.1 ml Matrigel (1:1) for tumor development. One week after the tumor cell inoculation, mice were injected i.v. with PD-L1-proIL-15 or PD-1-proIL-15. The mice in the vehicle group were injected with PBS. Tumor volume were measured in two dimensions using a caliper, and the volumes were expressed in mm³ using the formula: V=0.5a× b² where a and b are the longest and shortest diameters of the tumor, respectively. The tumor volume was then used for calculations TGI and T/C values. Tumor growth inhibition (TGI %) and relative tumor growth inhibition (T/C %) was calculated according to the following equation:

TGI%=(1−(Tₙ−T₀)/(Vₙ−V₀))×100% T/C%=(Tₙ/T₀)/(Vₙ/V₀)×100%

In the formula, Tₙ and Vₙ stands for tumor volume of treatment group and vehicle control group of day n after the start of treatment, respectively. T₀ and V₀ stands for tumor volume of corresponding groups on the day of grouping. Results were analyzed using Prism GraphPad.

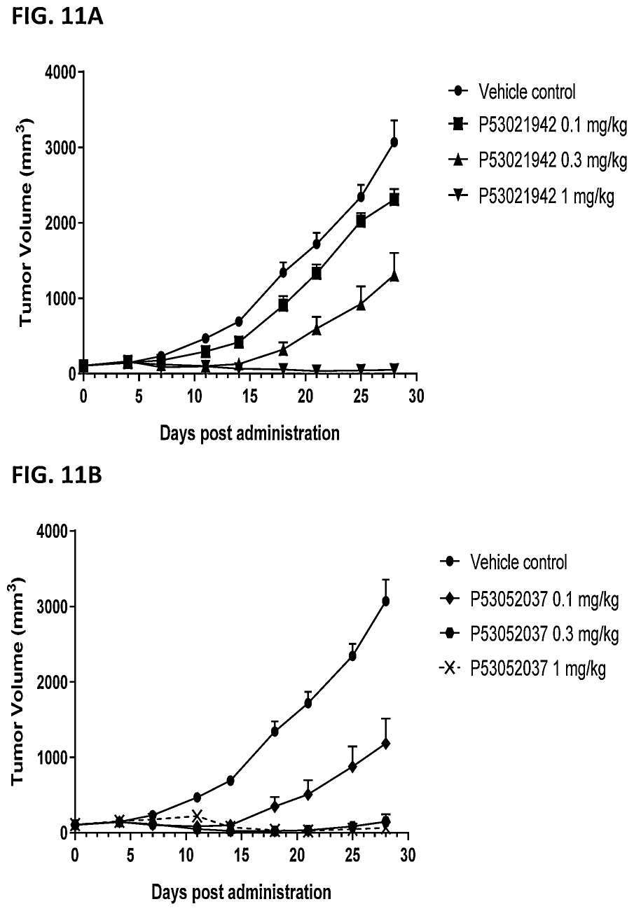

FIG. 11A and Table 2 show that PD-L1-proIL-15 mediated dose-dependent anti-tumor efficacy in A375-PBMC xenograft model, and FIG. 11B and Table 2 show that PD-1-proIL-15 also mediated dose-dependent anti-tumor efficacy in A375-PBMC xenograft model.

TABLE 2
Tumor volume of the mice treated with PD-L1-proIL-15
and PD-1-proIL-15 in A375-PBMC xenograft model

| | Animal | Tumor volume (mm³) | T/C | TGI |
|---|---|---|---|---|
| Treatment | No. | at day 28 | (%) | (%) |
| Vehicle control | 6 | 3070 ± 287 | — | — |
| P53021942 0.1 mg/kg | 6 | 2309 ± 139 | 75.21 | 25.68 |
| P53021942 0.3 mg/kg | 6 | 1308 ± 292 | 42.61 | 59.47 |
| P53021942 1 mg/kg | 6 | 51 ± 25 | 1.66 | 101.89 |
| P53052037 0.1 mg/kg | 6 | 1183 ± 330 | 38.53 | 63.69 |
| P53052037 0.3 mg/kg | 6 | 149 ± 95 | 4.85 | 98.58 |
| P53052037 1 mg/kg | 6 | 67 ± 64 | 2.18 | 101.35 |

### Example 6. In Vivo Efficacy of mPD-1-proIL-15 in B16F10 Syngeneic Model

The mPD-1-proIL-15 was tested for its anti-tumor efficacy in the B16F10 syngeneic model. The B16F10 cells were maintained in vitro in DMEM added with 10% FBS, 100 U/ml penicillin and 100 μg/ml streptomycin at 37° C. in an atmosphere of 5% CO₂ in air.

C57/B6 mice aged 6-8 weeks at the start of the experiment (Shanghai Lingchang Biotechnology Co., Ltd., Shanghai, China) were maintained under specific-pathogen-free condition with daily cycles of 12 h light/12 h darkness. The mice were kept in individual ventilation cages at constant temperature (20-26° C.) and humidity (40-70%) with ≤6 animals in each cage. Animals had free access to irradiation sterilized dry granule food and sterile drinking water during the entire study period. All the procedures related to animal handling, care and the treatment in this study were performed according to the guidelines approved by the Institutional Animal Care and Use Committee (IACUC) of Shanghai Viva prior to conduct. After arrival, animals were maintained for at least 3 days to get accustomed to the new environment. At the time of routine monitoring, the animals were checked for any effects of tumor growth on normal behavior such as mobility, food and water consumption (by looking only), body weight gain/loss (body weights were measured twice weekly), eye/hair matting and any other abnormal effect.

Mice were inoculated subcutaneously at the right flank with 2×10⁵ B16F10 cells in 0.1 ml HBSS for tumor development. 8 days after the tumor cell inoculation, mice were injected i.v. with mPD-1-proIL-15. The mice in the vehicle group were injected with PBS. Tumor volume were measured in two dimensions using a caliper, and the volumes were expressed in mm³ using the formula: V=0.5a× b² where a and b are the longest and shortest diameters of the tumor, respectively. The tumor volume was then used for calculations TGI and T/C values. Tumor growth inhibition (TGI %) and relative tumor growth inhibition (T/C %) was calculated according to the following equation:

TGI%=(1−(Tₙ−T₀)/(Vₙ−V₀))×100% T/C%=(Tₙ/T₀)/(Vₙ/V₀)×100%

In the formula, Tₙ and Vₙ stands for tumor volume of treatment group and vehicle control group of day n after the start of treatment, respectively. T₀ and V₀ stands for tumor volume of corresponding groups on the day of grouping. Results were analyzed using Prism GraphPad.

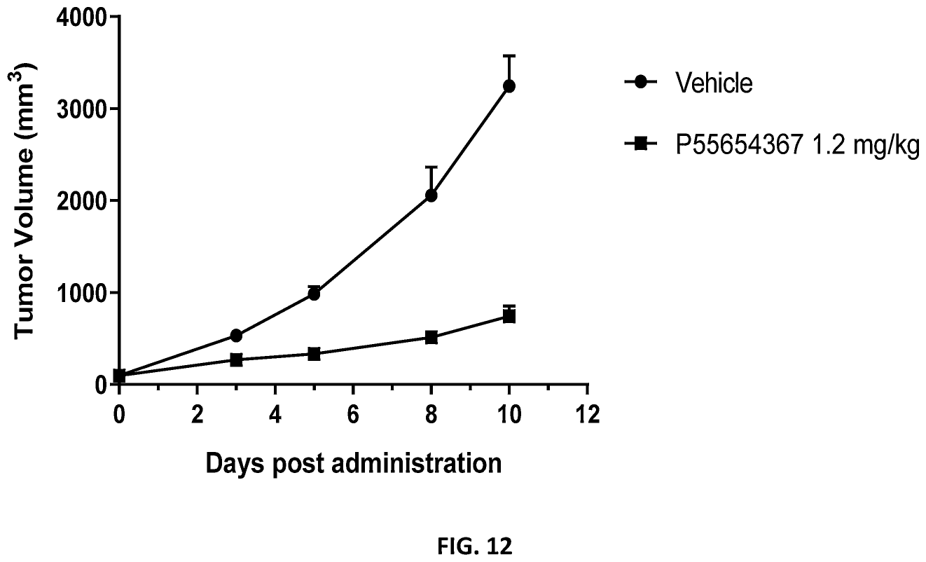

FIG. 12 and Table 3 show that mPD-1-proIL-15 significantly inhibited tumor growth at a dose of 1.2 mg/kg in the B16F10 syngeneic model.

TABLE 3
Tumor volume of the mice treated with mPD-
1-proIL-15 in B16F10 syngeneic model

| | Animal | Tumor volume (mm³) | T/C | TGI |
|---|---|---|---|---|
| Treatment | No. | at day 10 | (%) | (%) |
| Vehicle control | 6 | 3245 ± 329 | — | — |
| P55654367 1.2 mg/kg | 6 | 742 ± 110 | 22.87 | 79.54 |

### Example 7. Bioactivity of PD-1-proIL-15 in Cynomolgus Monkeys

The in vivo bioactivity of the PD-1-proIL-15 was assessed in cynomolgus monkeys. 1 male and 1 female cynomolgus monkey received a 2 repeat dose of P53052037 at 10 mg/kg.

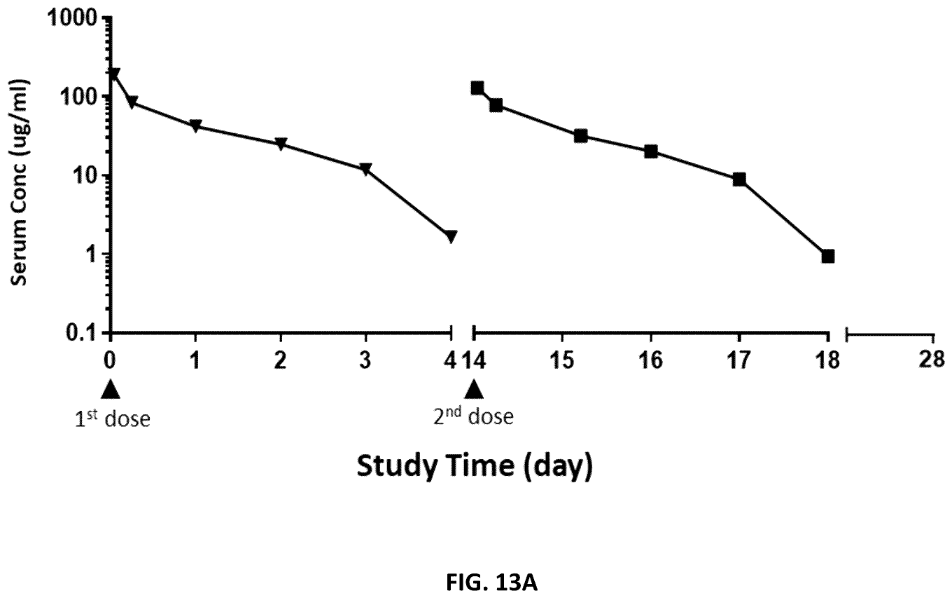

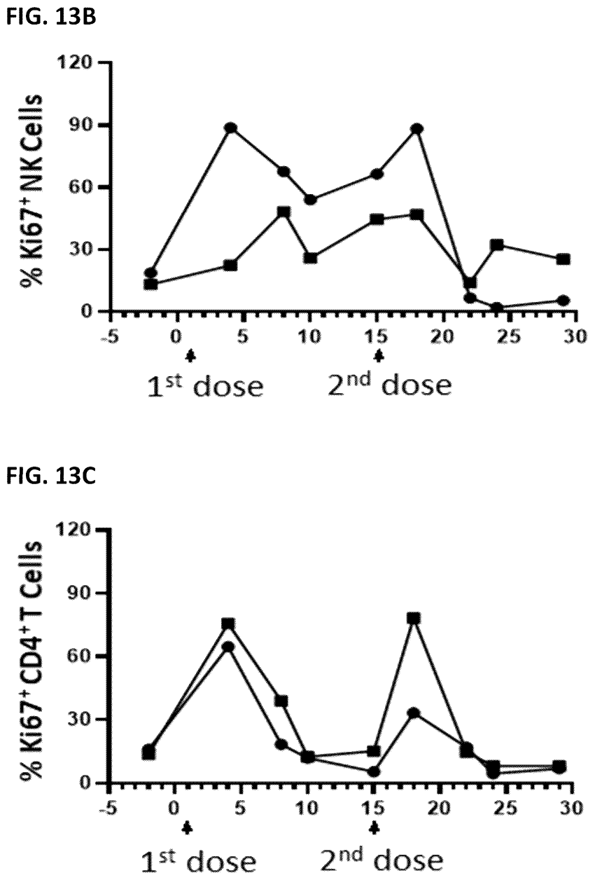

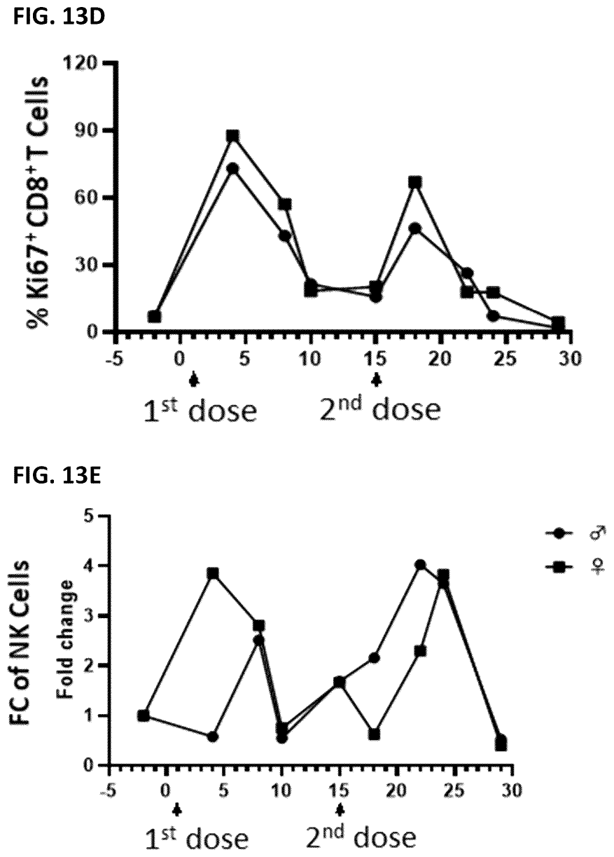

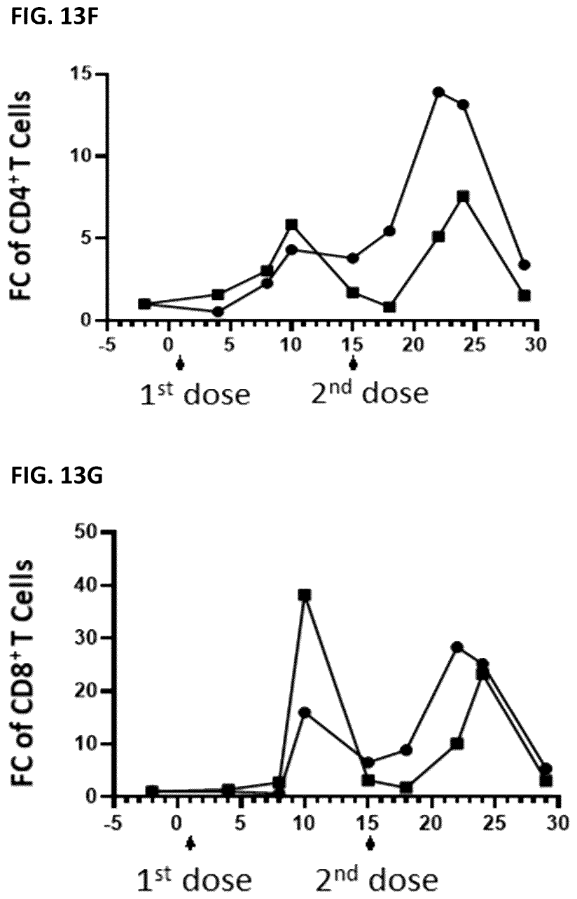

Blood samples for pharmacodynamics study in cynomolgus monkeys were collected before dosing and 1 h, 6 hrs, 24 hrs, 48 hrs, 72 hrs and 96 hrs after the 1^{st} dosing and 2^{nd} dosing. The concentration of P53052037 was determined using ELISAs utilizing PD-1 as the capture antibody and HRP conjugated anti-human IgG as the detection antibody. FIG. 13A shows that the concentrations of P53052037 in serum in cynomolgus monkeys, which demonstrated the T_{1/2} of P53052037 was extended, compared to IL-15.

Blood samples for immune cell subtype analysis in cynomolgus monkeys were collected before dosing and 4, 8, 10, 15, 18, 22, 24 and 29 days post-dosing. Cells were stained with stain buffer including CD45 (clone D058-1283, BD Pharmingen), CD3 (clone SP34-2, BD Pharmingen), CD4 (clone L200, BD Pharmingen), CD8 (clone RPA-T8, BioLegend) and CD56 (clone HCD56, BioLegend) for 30 min at 4° C. Then cells were fixed and permeabilized with Transcription Factor buffer (True-Nuclear Transcription Factor Buffer, BioLegend) and then incubated with Ki67 (clone B56, BD Pharmingen) for 45 min at RT.

Samples were analyzed using a flow cytometer (CytoFLEX S, Beckman coulter) gating on CD45⁺CD3⁻CD56⁺ cells (NK cells), CD45⁺CD3⁺CD4⁺ cells (CD4 T cells), CD45⁺CD3⁺CD8⁺ cells (CD8 T cells) and Ki67⁺ cells in NK, CD4⁺ T cells and CD8⁺ T cells. FIGS. 13B-13G show that the expression of Ki67 and the fold changes of the cell number in NK, CD4⁺ T cells and CD8⁺ T cells were increased after P53052037 10 mg/kg treatment, and later decreased to normal levels over time. All the monkeys were well tolerant after treated with P53052037 10 mg/kg.

1. An activatable proprotein homodimer, comprising a first polypeptide and a second polypeptide, wherein the first polypeptide and the second polypeptide comprise, in an N- to C-terminal orientation,
a fragment antigen-binding (Fab) region that specifically binds to human PD-1 or human PD-L1 or human B7H3, a hinge/Fc domain, a first linker, an IL-15 protein, a second linker, and an IL-15Rα protein,
wherein the hinge/Fc domain of the first polypeptide binds to the hinge/Fc domain of the second polypeptide, wherein the IL-15 protein of the first polypeptide binds to the IL-15Rα protein of the second polypeptide, and wherein the IL-15Rα of the first polypeptide binds to the IL-15 protein of the second polypeptide, wherein said binding masks a binding site of the IL-15 protein(s) that otherwise binds to an IL-15Rβ/γc chain present on the surface of an immune cell in vitro or in vivo,
and wherein the second linker is a cleavable linker.

2. The activatable proprotein homodimer of claim 1, wherein the Fab region specifically binds to human PD-1, and optionally comprises the Fab region from an anti-PD-1 antibody selected from nivolumab, pembrolizumab, cemiplimab, JTX-4014, spartalizumab, camrelizumab, sintilimab, tislelizumab, toripalimab, dostarlimab, MGA012, AMP-22, and AMP-514.

3. The activatable proprotein homodimer of claim 2, wherein the Fab region specifically binds to human PD-1 and comprises
a heavy chain variable (VH) region comprising VHCDR1, VHCDR2, and VHCDR3 regions set forth in SEQ ID NO: 1; and a light chain variable (VL) region comprising VLCDR1, VLCDR2, and VLCDR3 regions set forth in SEQ ID NO: 2;
a heavy chain variable (VH) region comprising VHCDR1, VHCDR2, and VHCDR3 regions set forth in SEQ ID NO: 3; and a light chain variable (VL) region comprising VLCDR1, VLCDR2, and VLCDR3 regions set forth in SEQ ID NO: 4;
a heavy chain variable (VH) region comprising VHCDR1, VHCDR2, and VHCDR3 regions set forth in SEQ ID NO: 5; and a light chain variable (VL) region comprising VLCDR1, VLCDR2, and VLCDR3 regions set forth in SEQ ID NO: 6; or
a heavy chain variable (VH) region comprising VHCDR1, VHCDR2, and VHCDR3 regions set forth in SEQ ID NO: 7; and a light chain variable (VL) region comprising VLCDR1, VLCDR2, and VLCDR3 regions set forth in SEQ ID NO: 8;
a heavy chain variable (VH) region comprising VHCDR1, VHCDR2, and VHCDR3 regions set forth in SEQ ID NO: 9; and a light chain variable (VL) region comprising VLCDR1, VLCDR2, and VLCDR3 regions set forth in SEQ ID NO: 10;
a heavy chain variable (VH) region comprising VHCDR1, VHCDR2, and VHCDR3 regions set forth in SEQ ID NO: 11; and a light chain variable (VL) region comprising VLCDR1, VLCDR2, and VLCDR3 regions set forth in SEQ ID NO: 12;
a heavy chain variable (VH) region comprising VHCDR1, VHCDR2, and VHCDR3 regions set forth in SEQ ID NO: 13; and a light chain variable (VL) region comprising VLCDR1, VLCDR2, and VLCDR3 regions set forth in SEQ ID NO: 14;
a heavy chain variable (VH) region comprising VHCDR1, VHCDR2, and VHCDR3 regions set forth in SEQ ID NO: 15; and a light chain variable (VL) region comprising VLCDR1, VLCDR2, and VLCDR3 regions set forth in SEQ ID NO: 16;
a heavy chain variable (VH) region comprising VHCDR1, VHCDR2, and VHCDR3 regions set forth in SEQ ID NO: 17; and a light chain variable (VL) region comprising VLCDR1, VLCDR2, and VLCDR3 regions set forth in SEQ ID NO: 18;
a heavy chain variable (VH) region comprising VHCDR1, VHCDR2, and VHCDR3 regions set forth in SEQ ID NO: 19; and a light chain variable (VL) region comprising VLCDR1, VLCDR2, and VLCDR3 regions set forth in SEQ ID NO: 20;
a heavy chain variable (VH) region comprising VHCDR1, VHCDR2, and VHCDR3 regions set forth in SEQ ID NO: 21; and a light chain variable (VL) region comprising VLCDR1, VLCDR2, and VLCDR3 regions set forth in SEQ ID NO: 22; or
a heavy chain variable (VH) region comprising VHCDR1, VHCDR2, and VHCDR3 regions set forth in SEQ ID NO: 23; and a light chain variable (VL) region comprising VLCDR1, VLCDR2, and VLCDR3 regions set forth in SEQ ID NO: 24.

4. The activatable proprotein homodimer of claim 3, wherein the VH region comprises an amino acid sequence having at least 90%, 95%, 96%, 97%, 98%, 99%, or 100% identity to a sequence selected from SEQ ID NOs: 1, 3, 5, 7, 9, 11, 13, 14, 15, 17, 19, 21, and 23, and the VL region respectively comprises an amino acid sequence having at least 90%, 95%, 96%, 97%, 98%, 99%, or 100% identity to a sequence selected from SEQ ID NOs: 2, 4, 6, 8, 10, 12, 14, 16, 18, 20, 22, and 24.

5. The activatable proprotein homodimer of claim 1, wherein the Fab region specifically binds to human PD-L1, and optionally comprises the Fab region from an anti-PD-L1 antibody selected from atezolizumab, avelumab, and durvalumab.

6. The activatable proprotein homodimer of claim 5, wherein the Fab region specifically binds to human PD-L1 and comprises
a heavy chain variable (VH) region comprising VHCDR1, VHCDR2, and VHCDR3 regions set forth in SEQ ID NO: 25; and a light chain variable (VL) region comprising VLCDR1, VLCDR2, and VLCDR3 regions set forth in SEQ ID NO: 26;
a heavy chain variable (VH) region comprising VHCDR1, VHCDR2, and VHCDR3 regions set forth in SEQ ID NO: 27; and a light chain variable (VL) region comprising VLCDR1, VLCDR2, and VLCDR3 regions set forth in SEQ ID NO: 28;
a heavy chain variable (VH) region comprising VHCDR1, VHCDR2, and VHCDR3 regions set forth in SEQ ID NO: 29; and a light chain variable (VL) region comprising VLCDR1, VLCDR2, and VLCDR3 regions set forth in SEQ ID NO: 30; or
a heavy chain variable (VH) region comprising VHCDR1, VHCDR2, and VHCDR3 regions set forth in SEQ ID NO: 31; and a light chain variable (VL) region comprising VLCDR1, VLCDR2, and VLCDR3 regions set forth in SEQ ID NO: 32;
a heavy chain variable (VH) region comprising VHCDR1, VHCDR2, and VHCDR3 regions set forth in SEQ ID NO: 33; and a light chain variable (VL) region comprising VLCDR1, VLCDR2, and VLCDR3 regions set forth in SEQ ID NO: 34;
a heavy chain variable (VH) region comprising VHCDR1, VHCDR2, and VHCDR3 regions set forth in SEQ ID NO: 35; and a light chain variable (VL) region comprising VLCDR1, VLCDR2, and VLCDR3 regions set forth in SEQ ID NO: 36; or
a heavy chain variable (VH) region comprising VHCDR1, VHCDR2, and VHCDR3 regions set forth in SEQ ID NO: 37; and a light chain variable (VL) region comprising VLCDR1, VLCDR2, and VLCDR3 regions set forth in SEQ ID NO: 38.

7. The activatable proprotein homodimer of claim 6, wherein the VH region comprises an amino acid sequence having at least 90%, 95%, 96%, 97%, 98%, 99%, or 100% identity to a sequence selected from SEQ ID NOs: 25, 27, 29, 31, 33, 35, and 37, and the VL region respectively comprises an amino acid sequence having at least 90%, 95%, 96%, 97%, 98%, 99%, or 100% identity to a sequence selected from SEQ ID NOs: 26, 28, 30, 32, 34, 36, and 38.

8. The activatable proprotein homodimer of claim 1, wherein the Fab region specifically binds to human B7H3 and comprises a heavy chain variable (VH) region comprising VHCDR1, VHCDR2, and VHCDR3 regions set forth in SEQ ID NO: 202; and a light chain variable (VL) region comprising VLCDR1, VLCDR2, and VLCDR3 regions set forth in SEQ ID NO: 203.

9. The activatable proprotein homodimer of claim 8, wherein the VH region comprises an amino acid sequence having at least 90%, 95%, 96%, 97%, 98%, 99%, or 100% identity to SEQ ID NO: 202, and the VL region respectively comprises an amino acid sequence having at least 90%, 95%, 96%, 97%, 98%, 99%, or 100% identity to SEQ ID NO: 203.

10. The activatable proprotein homodimer of claim 1, wherein the Fc domain comprises a CH2 domain, a CH3 domain, or a CH2CH3 domain of an immunoglobulin, optionally wherein the immunoglobulin is from an immunoglobulin class selected from IgG1, IgG2, IgG3, IgG4, IgA, IgD, IgE, and IgM.

11. The activatable proprotein homodimer of claim 1, wherein the hinge comprises an amino acid sequence having at least 90%, 95%, 96%, 97%, 98%, 99%, or 100% identity to a sequence selected from Table F1, and wherein the Fc domain comprises an amino acid sequence having at least 90%, 95%, 96%, 97%, 98%, 99%, or 100% identity to a sequence selected from Table F1.

12. The activatable proprotein homodimer of claim 1, wherein the Fc domain is a modified Fc domain that does not bind or substantially bind to FcγR, and retains normal or substantially normal binding to FcRn.

13. The activatable proprotein homodimer of claim 12, wherein the modified Fe domain comprises a modified IgG1 CH2 domain with the L234A/L235A (“LALA”) mutations and/or the P329A or P329G mutation (EU numbering).

14. The activatable proprotein homodimer of claim 1, wherein the IL-15 protein comprises, consists, or consists essentially of an amino acid sequence that is at least 80, 85, 90, 95, 98, or 100% identical to a sequence selected from Table S1, optionally wherein the IL-15 protein comprises, consists, or consists essentially of an amino acid sequence that is at least 80, 85, 90, 95, 98, or 100% identical to SEQ ID NO: 79 which retains the K86G and S162A mutations.

15. The activatable proprotein homodimer of claim 14, wherein the IL-15 protein comprises or retains one or more amino acid substitutions at position D8, D22, E46, V49, 150, L66, and/or K86 as defined by SEQ ID NO: 69 (mature human IL-15), and/or S162 as defined by SEQ ID NO: 68 (IL-15 FL precursor).

16. The activatable proprotein homodimer of claim 15, wherein the one or more amino acid substitutions are selected from D8N, D22K, E46K, V49D, I50D, L66E, K86G, and 162A, optionally the combination of K86G and S162A.

17. The activatable proprotein homodimer of claim 16, wherein the one or more amino acid substitutions are combinations selected from K86G and 162A, V49D and S162A; I50D and S162A; L66E and S162A; D8N and S162A; V49D and S162A; E46K and S162A; E46K, E53K, and S162A; D22K, E46K, and S162A; and D22K, E46K, E53K, and S162A.

18. The activatable proprotein homodimer of claim 1, wherein the IL-15Rα protein comprises, consists, or consists essentially of an amino acid sequence that is at least 80, 85, 90, 95, 98, or 100% identical to a sequence selected from Table S2, optionally wherein the IL-15Rα protein comprises, consists, or consists essentially of an amino acid sequence that is at least 80, 85, 90, 95, 98, or 100% identical to SEQ ID NO: 87 or 88 and retains the T2A substitution.

19. The activatable proprotein homodimer of claim 18, wherein the IL-15Rα protein comprises or retains one or more amino acid substitutions selected from R24E, R26E, as defined by SEQ ID NO: 82 (IL-15Rα Sushi+).

20-21. (canceled)

22. The activatable proprotein homodimer of claim 18, wherein the IL-15Rα protein comprises or retains an amino acid substitutions at position T2 as defined by SEQ ID NO: 82 (IL-15Rα Sushi+).

23. The activatable proprotein homodimer of claim 22, wherein the amino acid substitution is T2A.

24. The activatable proprotein homodimer of claim 23, wherein the IL-15a protein comprises SEQ ID NO: 82 or 83 with the T2A substitution.

25. The activatable proprotein homodimer of claim 1, wherein the hinge of the first polypeptide forms at least one or two disulfide bonds with the hinge of the second polypeptide.

26. The activatable proprotein homodimer of claim 1, wherein the first linker is a non-cleavable or stable linker, and wherein the cleavable linker comprises a protease cleavage site, optionally wherein the cleavable linker is selected from Table S3.

27-29. (canceled)

30. The activatable proprotein homodimer of claim 1, wherein the Fab comprises SEQ ID NOs: 3 (VH) and a human IgG1 CH1 domain, and SEQ ID NO:4 (VL) and a CL domain (human kappa); the Fc domain comprises the IgG1 hinge of SEQ ID NO: 42, a modified human IgG1 CH2 domain of SEQ ID NO: 57, and a human IgG1 CH3 domain of SEQ ID NO: 58; the first linker is an 8 amino acid stable linker of SEQ ID NO: (178, wherein x is 2); the IL-15 protein comprises SEQ ID NO: 79, optionally with the K86G and S162A mutations; the second linker is a protease cleavable linker of SEQ ID NO: 90 or SEQ ID NO: 201; and the IL-15Rα protein comprises SEQ ID NO: 87, optionally with a T2A mutation.

31. The activatable proprotein homodimer of claim 1, wherein cleavage, optionally protease cleavage, of the second linker exposes the binding site(s) of the IL-15 proteins that bind to the IL-15Rβ/γc chain present on the surface of the immune cell in vitro or in vivo.

32. (canceled)

33. The activatable proprotein homodimer of claim 1, wherein the first polypeptide and the second polypeptide comprise, consist, or consist essentially of an amino acid sequence that is at least 80, 85, 90, 95, 98, or 100% identical to a sequence selected from Table S4 (chains 1 and 2), and a VL/CL region polypeptide that is at least 80, 85, 90, 95, 98, or 100% identical to the corresponding sequence from Table S4 (chains 3 and 4), optionally wherein:
the first polypeptide and the second polypeptide comprise, consist, or consist essentially of an amino acid sequence that is at least 80, 85, 90, 95, 98, or 100% identical to SEQ ID NO: 136, and a VL/CL region polypeptide that is at least 80, 85, 90, 95, 98, or 100% identical to SEQ ID NO: 137;
the first polypeptide and the second polypeptide comprise, consist, or consist essentially of an amino acid sequence that is at least 80, 85, 90, 95, 98, or 100% identical to SEQ ID NO: 138, and a VL/CL region polypeptide that is at least 80, 85, 90, 95, 98, or 100% identical to SEQ ID NO: 139;
the first polypeptide and the second polypeptide comprise, consist, or consist essentially of an amino acid sequence that is at least 80, 85, 90, 95, 98, or 100% identical to SEQ ID NO: 140, and a VL/CL region polypeptide that is at least 80, 85, 90, 95, 98, or 100% identical to SEQ ID NO: 141;
the first polypeptide and the second polypeptide comprise, consist, or consist essentially of an amino acid sequence that is at least 80, 85, 90, 95, 98, or 100% identical to SEQ ID NO: 142, and a VL/CL region polypeptide that is at least 80, 85, 90, 95, 98, or 100% identical to SEQ ID NO: 143;
the first polypeptide and the second polypeptide comprise, consist, or consist essentially of an amino acid sequence that is at least 80, 85, 90, 95, 98, or 100% identical to SEQ ID NO: 144, and a VL/CL region polypeptide that is at least 80, 85, 90, 95, 98, or 100% identical to SEQ ID NO: 145;
the first polypeptide and the second polypeptide comprise, consist, or consist essentially of an amino acid sequence that is at least 80, 85, 90, 95, 98, or 100% identical to SEQ ID NO: 146, and a VL/CL region polypeptide that is at least 80, 85, 90, 95, 98, or 100% identical to SEQ ID NO: 147;
the first polypeptide and the second polypeptide comprise, consist, or consist essentially of an amino acid sequence that is at least 80, 85, 90, 95, 98, or 100% identical to SEQ ID NO: 148, and a VL/CL region polypeptide that is at least 80, 85, 90, 95, 98, or 100% identical to SEQ ID NO: 149; or
the first polypeptide and the second polypeptide comprise, consist, or consist essentially of an amino acid sequence that is at least 80, 85, 90, 95, 98, or 100% identical to SEQ ID NO: 199, and a VL/CL region polypeptide that is at least 80, 85, 90, 95, 98, or 100% identical to SEQ ID NO: 200.

34. (canceled)

35. One or more recombinant nucleic acid molecules that encode the activatable proprotein homodimer of claim 1.

36. The one or more recombinant nucleic acid molecules of claim 35, wherein a first recombinant nucleic acid molecule encodes the VH/CH1 regions of the Fab region, the hinge/Fc domain, the first linker, the IL-15 protein, the second linker, and the IL-15Rα protein, and wherein a second nucleic acid molecule encodes the VL/CL regions of the Fab region.

37. One or more vectors comprising the one or more recombinant nucleic acid molecules of claim 35.

38. A host cell comprising the one or more vectors of claim 37.

39. A method of producing an activatable proprotein, comprising culturing the host cell of claim 38 under culture conditions suitable for the expression of the activatable proprotein homodimer, and isolating the activatable proprotein from the culture.

40. A pharmaceutical composition, comprising the activatable proprotein homodimer of claim 1, and a pharmaceutically acceptable carrier.

41. A method of treating disease in a subject, and/or a method of enhancing an immune response in a subject, comprising administering to the subject a therapeutically effective amount of the pharmaceutical composition of claim 40.

42. The method of claim 41, wherein the disease is a cancer, optionally a cancer that expresses or over-expresses PD-L1 or B7H3.

43. The method of claim 42, wherein the cancer is a primary cancer or a metastatic cancer, and is selected from one or more of melanoma (optionally metastatic melanoma), kidney cancer (optionally renal cell carcinoma), pancreatic cancer, bone cancer, prostate cancer, small cell lung cancer, non-small cell lung cancer (NSCLC), mesothelioma, leukemia (optionally lymphocytic leukemia, chronic myelogenous leukemia, acute myeloid leukemia, or relapsed acute myeloid leukemia), multiple myeloma, lymphoma, hepatoma (hepatocellular carcinoma), sarcoma, B-cell malignancy, breast cancer, ovarian cancer, colorectal cancer, glioma, glioblastoma multiforme, meningioma, pituitary adenoma, vestibular schwannoma, primary CNS lymphoma, primitive neuroectodermal tumor (medulloblastoma), bladder cancer, uterine cancer, esophageal cancer, brain cancer, head and neck cancers, cervical cancer, testicular cancer, thyroid cancer, and stomach cancer.

44. The method of claim 41, wherein following administration, the activatable proprotein homodimer is activated through protease cleavage in a cancer cell or cancer tissue, or a tumor microenvironment (TME), which exposes the binding site(s) of the IL-15 proteins that bind to the IL-15Rβ/γc chain present on the surface of the immune cell in vitro or in vivo, and thereby generates an activated protein.

45-52. (canceled)
